SEQUENCE LISTING (11 sequences)

SEQ ID NO: 1 - DNA, 1467 bp, Rothia mucilaginosa
ctggctcaggacgaacgctggcggcgtgcttaacacatgcaagtcgaacgatgaaccctagcttgctaggtggattagtggcgaacgggtgagtaacacgtgagtaacctacccttaactctgggataagcctgggaaactgggtctaataccggatacgaccaatctccgcatggggtgttggtggaaagcgttatgtagtggttatagatgggctcacggcctatcagcttgttggtgaggtaacggctcaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctgttagcagggaagaagagagattgacggtacctgcagagaaagcgccggctaactacgtgccagcagccgcggtaatacgtagggcgcgagcgttgtccggaattattgggcgtaaagagcttgtaggcggtttgtcgcgtctgctgtgaaaggccggggcttaaccccgtgtattgcagtgggtacgggcagactagagtgcagtaggggagactggaattcctggtgtagcggtggaatgcgcagatatcaggaagaacaccgatggcgaaggcaggtctctgggctgtaactgacgctgagaagcgaaagcatggggagcgaacaggattagataccctggtagtccatgccgtaaacgttgggcactaggtgtgggggacattccacgttttccgcgccgtagctaacgcattaagtgccccgcctggggagtacggccgcaaggctaaaactcaaagaaattgacgggggcccgcacaagcggcggagcatgcggattaattcgatgcaacgcgaagaaccttaccaaggcttgacatatactggaccgcatcagagatggtgtttcccttcggggctggtatacaggtggtgcatggttgtcgtcagctcgtgtcgtgagatgttgggttaagtcccgcaacgagcgcaaccctcgttctatgttgccagcacgttatggtggggactcataggagactgccggggtcaactcggaggaaggtggggatgacgtcaaatcatcatgccccttatgtcttgggcttcacgcatgctacaatggccggtacagagggttgcgatactgtgaggtggagctaatccctaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagttggtaacacccaaagccggtggcctaacctcttttgggagggagccgtctaaggtgggatcggcgattgggactaagtcg

SEQ ID NO: 2 - DNA, 1515 bp, Rothia dentocariosa
agagtttgattctggctcaggacgaacgctggcggcgtgcttaacacatgcaagtcgaacgatgaagcctagcttgctaggtggattagtggcgaacgggtgagtaatacgtgagtgacctacctttgactctgggataagcctgggaaactgggtctaataccggatacgaccaatctccgcatggggtgttggtggaaagcgttatggagtggttttagatgggctcacggcctatcagcttgttggtgaggtaatggcttaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctgttagcatcgaagaagcgaaagtgacggtaggtgcagagaaagcgccggctaactacgtgccagcagccgcggtaatacgtagggcgcgagcgttgtccggaattattgggcgtaaagagcttgtaggcggttggtcgcgtctgctgtgaaaggctggggcttaaccctggttttgcagtgggtacgggctaactagagtgcagtaggggagactggaattcctggtgtagcggtggaatgcgcagatatcaggaggaacaccgatggcgaaggcaggtctctgggctgtaactgacgctgagaagcgaaagcatggggagcgaacaggattagataccctggtagtccatgccgtaaacgttgggcactaggtgtgggggacattccacgttttccgcgccgtagctaacgcattaagtgccccgcctggggagtacggccgcaaggctaaaactcaaagaaattgacgggggcccgcacaagcggcggagcatgcggattaattcgatgcaacgcgaagaaccttaccaaggcttgacatatactggactgcgtcagagatggcgtttcccttcggggctggtatacaggtggtgcatggttgtcgtcagctcgtgtcgtgagatgttgggttaagtcccgcaacgagcgcaaccctcgttctatgttgccagcacgtgatggtggggactcataggagactgccggggtcaactcggaggaaggtggggatgacgtcaaatcatcatgccccttatgtcttgggcttcacgcatgctacaatggccggtacagagggttgcgatactgtgaggtggagctaatccctaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagttggtaacacccgaagccggtggcctaaccctggtggggggagccgtcgaaggtgggactggcgattgggactaagtcgtaacaaggtagccgtaccggaaggtgcggctggatcacct

SEQ ID NO: 3 - DNA, 1436 bp, Rothia terrae
acatgcagtcgaacgatgaagaccggtgcttgcactggttggattagtggcgaacgggtgagtaatacgtgagtaacctgcctttgactctgggataagccttggaaacgaggtctaataccggatatgacacagtttcgcatgaaatctgtgtggaaagggttttgtactggttttagatgggctcacggcctatcagcttgttggtgaggtaatggctcaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctttcagcaggggagaagcgaaagtgacggtacctgcagaagaagcgccggctaactacgtgccagcagccgcggtaatacgtagggcgcgagcgttgtccggaattattgggcgtaaagagcttgtaggcggtttgtcgcgtctgctgtgaaagcccggggcttaactccgggtttgcagtgggtacgggctaactagagtgcagtaggggagactggaattcctggtgtagcggtggaatgcgcagatatcaggaggaacaccgatggcgaaggcaggtctctgggctgtaactgacgctgagaagcgaaagcatggggagcgaacaggattagataccctggtagtccatgccgtaaacgttgggcactaggtgtgggggacattccacgttttccgcgccgtagctaacgcattaagtgccccgcctggggagtacggccgcaaggctaaaactcaaagaaattgacgggggcccgcacaagcggcggagcatgcggattaattcgatgcaacgcgaagaaccttaccaaggcttgacatatactggactcccctagagatagggtttcccttcggggctggtatacaggtggtgcatggttgtcgtcagctcgtgtcgtgagatgttgggttaagtcccgcaacgagcgcaaccctcgttctatgttgccagcacgtgatggtggggactcataggagactgccggggtcaactcggaggaaggtggggatgacgtcaaatcatcatgccccttatgtcttgggcttcacgcatgctacaatggccggtacaaagggttgcgatactgtgaggttgagctaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagttggtaacacccgaagccgatggcctaacctttttggagggagtcgtcgaaggtgggactggcgattgggactaagtcgtaa

SEQ ID NO: 4 - DNA, 1467 bp, Rothia amarae
cagagtttgatctggctcaggacgaacgctggcggcgtgcttaacacatgcaagtcgaacgatgaagactggtgcttgcactggttggattagtggcgaacgggtgagtaatacgtgagtaacctgcctttgactctgggataagcctgggaaactgggtctaataccggatatgacaaggaaccgcatggttttttgtggaaagggtttgtactggttttagatgggctcacggcctatcagcttgttggtggggtaatggctcaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcgagcgttgtccggaattattgggcgtaaagagcttgtaggcggtttgttgcgtctgctgtgaaagaccggggcttaaccccggtattgcagtgggtacgggcagactagagtgcagtaggggagactggaattcctggtgtagcggtgaaatgcgcagatatcaggaggaacaccgatggcgaaggcaggtctctgggctgtaactgacgctgagaagcgaaagcatggggagcgaacaggattagataccctggtagtccatgccgtaaacgttgggcactaggtgtgggggacattccacgttttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatatactggaccgcctcagagatggggtttcccttcggggctggtatacaggtggtgcatggttgtcgtcagctcgtgtcgtgagatgtcagctcgtgtcgtgagatgttgggttaagtcccgcaacgagcgcaaccctcgttctatgttgccagcacgttgtggtggggactcataggagactgccggggtcaactcggaggaaggtggggatgacgtcaaatcatcatgccccttatgtcttgggcttcacgcatgctacaatggccggtacaaagggttgcgatactgtgaggttgagctaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagttggtaacacccgaagccgatggcctaaccccttgtgggagggagtcgt

SEQ ID NO: 5 - DNA, 1482 bp, Roseomonas gilardii
tagtttatcctggctcagagcgaacgctggcggcatgcttaacacatgcaagtcgcacgggtggtttcggccatcagtggcggacgggtgagtaacgcgtaggaacgtgtccagaggtgggggacaaccccgggaaactggggctaataccgcatgggggctgaggcccaaagccgagaggcgccattggagcggcctgcgtccgattaggtagttggtggggtaaaggcctaccaagcctgcgatcggtagctggtctgagaggacgaccagccacactgggactgagacacggcccagactcctacgggaggcagcagtggggaatattggacaatgggcgaaagcctgatccagcaatgccgcgtgggtgaagaaggtcttcggatcgtaaagccctttcgacggggacgatgatgacggtacccgtagaagaagccccggctaacttcgtgccagcagccgcggtaatacgaagggggctagcgttgctcggaattactgggcgtaaagggcgcgtaggcggcggcccaagtcaggcgtgaaattcctgggctcaacctggggactgcgcttgatactgggttgcttgaggatggaagaggctcgtggaattcccagtgtagaggtgaaattcgtagatattgggaagaacaccggtggcgaaggcggcgagctggtccattactgacgctgaggcgcgacagcgtggggagcaaacaggattagataccctggtagtccacgccgtaaacgatgtgcgctggatgttggggcccatagggtctcagtgtcgtagccaacgcggtaagcgcaccgcctggggagtacggccgcaaggttgaaactcaaaggaattgacgggggcccgcacaagcggtggagcatgtggtttaattcgaagcaacgcgcagaaccttaccagcccttgacatggtcacgaccggtccggagacgggccttccccgcaaggggcgtgatgcacaggtgctgcatggctgtcgtcagctcgtgtcgtgagatgttgggttaagtcccgcaacgagcgcaaccctcgcctctagttgccagcatgttcgggtgggcactctagaggaactgccggtgacaagccggaggaaggtggggatgacgtcaagtcctcatggcccttatgggctgggctacacacgtgctacaatggcggtgacagagggaagccaggtcgcgaggccgagccgatcccgaaaagccgtctcagttcggattgcactctgcaactcgggtgcatgaaggtggaatcgctagtaatcgcggatcagcacgccgcggtgaatacgttcccgggccttgtacacaccgcccgtcacaccatgggagttggttctaccttaagtcgttgcgctaaccgcaagggggcaggcgaccacggtagggtcagcgactggggtgaagtcgtaacaaggtagccgtaggggaacctgcggttggatcaccttctaa

SEQ ID NO: 6 - DNA, 1482 bp, Roseomonas mucosa
ttagtttgatcctggctcagagcgaacgctggcggcatgcttaacacatgcaagtcgcacgggcagcaatgtcagtggcggacgggtgagtaacgcgtaggaacgtgtcctgaggtgggggacaaccccgggaaactggggctaataccgcatatgggctgaggcccaaagccgagaggcgcctttggagcggcctgcgtccgattaggtagttggtggggtaaaggcctaccaagcctgcgatcggtagctggtctgagaggacgaccagccacactgggactgagacacggcccagactcctacgggaggcagcagtggggaatattggacaatgggcgaaagcctgatccagcaatgccgcgtgggtgaagaaggtcttcggatcgtaaagccctttcgacggggacgatgatgacggtacccgtagaagaagccccggctaacttcgtgccagcagccgcggtaatacgaagggggctagcgttgctcggaattactgggcgtaaagggcgcgtaggcggcggcccaagtcaggcgtgaaattcctgggctcaacctggggactgcgcttgatactgggttgcttgaggatggaagaggctcgtggaattcccagtgtagaggtgaaattcgtagatattgggaagaacaccggtggcgaaggcggcgagctggtccattactgacgctgaggcgcgacagcgtggggagcaaacaggattagataccctggtagtccacgccgtaaacgatgtgcgctggatgttggggcccatagggtctcagtgtcgtagccaacgcggtaagcgcaccgcctggggagtacggccgcaaggttgaaactcaaaggaattgacgggggcccgcacaagcggtggagcatgtggtttaattcgaagcaacgcgcagaaccttaccagcccttgacatggtcacgaccggtccagagatggactttcctagcaataggcgtgatgcacaggtgctgcatggctgtcgtcagctcgtgtcgtgagatgttgggttaagtcccgcaacgagcgcaaccctcgcctctagttgccagcatgttcgggtgggcactctagaggaactgccggtgacaagccggaggaaggtggggatgacgtcaagtcctcatggcccttatgggctgggctacacacgtgctacaatggcggtgacagagggaagccaggtcgcgaggccgagccgatcccgaaaagccgtctcagttcggattgcactctgcaactcgggtgcatgaaggtggaatcgctagtaatcgcggatcagcacgccgcggtgaatacgttcccgggccttgtacacaccgcccgtcacaccatgggagttggttctaccttaagtcgttgcgctaaccagcgatgggggcaggcgaccacggtagggtcagcgactggggtgaagtcgtaacaaggtagccgtaggggaacctgcggttggatcacctcctaa

SEQ ID NO: 7 - DNA, 1537 bp, Gemella haemolysans
gatcctggctcaggacgaacgctggcggcgtgcctaatacatgcaagtcgagcgaagtttttctggtgcttgcaccggaaaaacttagcggcgaacgggtgagtaacacgtaaagaacctgcctcatagactgggacaactattggaaacgatagctaataccggataacagcattaactgcatggttgatgtttaaaagttggttttgctaacactatgagatggctttgcggtgcattagctagttggtggggtaaaggcctaccaaggcgacgatgcatagccgacctgagagggtgatcggccacactgggactgagacacggcccagactcctacgggaggcagcagtagggaatcttccgcaatgggcgaaagcctgacggagcaacgccgcgtgagtgaagaaggatttcggttcgtaaagctctgttgttagggaagaatgattgtgtagtaactatacacagtagagacggtacctaaccagaaagccacggctaactacgtgccagcagccgcggtaatacgtaggtggcaagcgttgtccggaattattgggcgtaaagcgcgcgcaggtggtttaataagtctgatgtgaaagcccacggctcaaccgtggagggtcattggaaactgttaaacttgagtgcaggagagaaaagtggaattcctagtgtagcggtgaaatgcgtagagattaggaggaacaccagtggcgaaggcggctttttggcctgtaactgacactgaggcgcgaaagcgtggggagcaaacaggattagataccctggtagtccacgccgtaaacgatgagtgctaagtgttggtctcataagagatcagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaagtcttgacatactgtgaggacacaagagattgtgttgttctgacctttggttagacacagatacaggtggtgcatggttgtcgtcagctcgtgtcgtgagatgttgggttaagtcccgcaacgagcgcaacccttatatctagttgccagcagtaagatggggactctagatagactgccagtgataaactggaggaaggtggggatgacgtcaaatcatcatgccccttatgacttgggctacacacgtgctacaatggataggaacaaagagaagcgagctcgcgagagtaagcaaacctcacaaaactattctcagttcggattgtagtctgcaactcgactacatgaagctggaatcgctagtaatcgcgaatcagaatgtcgcggtgaatacgttcccgggtcttgtacacaccgcccgtcacaccacgagagtttgtaacacccgaagacggtggcctaaccttttaggagggagccggtcacgtgggacagatgattggggtgaagtcgtaacaaggtagccgtatcggaaggtgcggctggatcacct

SEQ ID NO: 8 - DNA, 1508 bp, Gemella bergeri
gacgaacgctggcggcgtgcctaatacatgcaagtcgagcgaagaatttctggtgcttgcactagaaattcttagcggcgaacgggtgagtaacacgtaaagaacctgccttatagactgggataactacgggaaactgtagctaataccggataacaacattttccgcatgggagatgtttaaaagttggtgaagctaacactataagatggctttgcggagcattagctagttggtgggataaaagcctaccaaggcgacgatgcatagccgacctgagagggtgaacggccacattgggactgagacacggcccaaactcctacgggaggcagcagtagggaatcttccgcaatggacgaaagtctgacggagcaacgccgcgtgagtgaagaaggatttcggttcgtaaagctctgttgttagggaagaaaggttgtgtagtaactatacacaaaacagacggtacctaaccagaaagccacggctaactacgtgccagcagccgcggtaatacgtaggtggcaagcgttgtccggaattattgggcgtaaagcgcgcgcaggtggtttagaaagtctgatgtgaaagcccacggctcaaccgtggaaggtcattggaaactgataaacttgagtgcaggagagaaaagtggaattcctagtgtagcggtgaaatgcgtagagattaggaggaacaccagtggcgaaggcggctttttggcctgcaactgacactgaggcgcgaaagcgtggggagcaaacaggattagataccctggtagtccacgccgtaaacgatgagtgctaagtgttggtctcaaaagagatcagtgctgcagcaaacgcattaagcactccgcctggggagtacgaccgcaaggttgaaactcaaaggaattgacggggacccgcacaagcggtggagcatgtggtttaattcgaagcaacgcgaagaaccttaccaagtcttgacatactgtgaggacataagagattatgttgtttgtcttttttagacaacacagatacaggtggtgcatggttgtcgtcagctcgtgtcgtgagatgttgggttaagtcccgcaacgagcgcaacccttatatctagttgccagcagtaagatggggactctagatagactgccagtgacaaactggaggaaggtggggatgacgtcaaatcatcatgccccttatgacttgggctacacacgtgctacaatggatagggacaaagagaagcgaacttgtaaaggcaagccaatctcataaaactattctcagttcggattgtagtctgcaactcgactacatgaagctggaatcgctagtaatcgcgaatcagaatgtcgcggtgaatacgttcccgggtcttgtacacaccgcccgtcacaccacgagagtttgtaacacctgaagacggtgacctaaccgaaagggaggagccggtcacggtgggacagatgattggggtgaagtcgtaacaaggtagccgtatcggaagg

SEQ ID NO: 9 - DNA, 1536 bp, Gemella morbillorum
atcctggctcaggacgaacgctggcggcgtgcctaatacatgcaagtcgagcgaagtttttctggtgcttgcactagaaaaacttagcggcgaacgggtgagtaacacgtaaagaacctgcctcatagactgggacaactattggaaacgatagctaataccggataacagtatttctcgcatgagagatatttaaaagttggttatgctaacactatgagatggctttgcggtgcattagctagttggtggggtaaaggcccaccaaggcgacgatgcatagccgacctgagagggtgatcggccacactgggactgagacacggcccagactcctacgggaggcagcagtagggaatcttccgcaatgggcgaaagcctgacggagcaacgccgcgtgagtgaagaaggatttcggttcgtaaagctctgttgttagggaagaatggatatgtagtaactatacatgtaagagacggtacctaaccagaaagccacggctaactacgtgccagcagccgcggtaatacgtaggtggcaagcgttgtccggaattattgggcgtaaagcgcgcgcaggtggtttaataagtctgatgtgaaagcccacggctcaaccgtggagggtcattggaaactgttaaacttgagtgcaggagagaaaagtggaattcctagtgtagcggtgaaatgcgtagagattaggaggaacaccagtggcgaaggcggctttttggcctgtaactgacactgaggcgcgaaagcgtggggagcaaacaggattagataccctggtagtccacgccgtaaacgatgagtgctaagtgttggtctcataagagatcagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaagtcttgacatactgtgaggacacaagagattgtgttgttctgacctttggttagacacagatacaggtggtgcatggttgtcgtcagctcgtgtcgtgagatgttgggttaagtcccgcaacgagcgcaacccttatatctagttgccagcagtaagatggggactctagatagactgccagtgataaactggaggaaggtggggatgacgtcaaatcatcatgccccttatgacttgggctacacacgtgctacaatggataggaacaaagagaagcgagctcgcgagagtcagccaacctcataaaactattctcagttcggattgtagtctgcaactcgactacatgaagctggaatcgctagtaatcgcgaatcagaatgtcgcggtgaatacgttcccgggtcttgtacacaccgcccgtcacaccacgagagtttgtaacacccgaagacggtggcctaacctttaaggagggagccggtcacgtgggacagatgattggggtgaagtcgtaacaaggtagccgtatcggaaggtgcggctggatcacct

SEQ ID NO: 10 - DNA, 1510 bp, Gemella sanguinis
gacgaacgctggcggcgtgcctaatacatgcaagtcgagcgaagtttttctggtgcttgcactagaaaaacttagcggcgaacgggtgagtaacacgtaaagaacctgcctcatagacggggacaactattggaaacgatagctaataccggataacagcataaatcgcatgatatatgtttaaaagttggtttcggctaacactatgagatggctttgcggtgcattagctagttggtggggtaaaggcctaccaaggcgacgatgcatagccgacctgagagggtgatcggccacactgggactgagacacggcccagactcctacgggaggcagcagtagggaatcttccgcaatgggcgaaagcctgacggagcaacgccgcgtgagtgaagaaggatttcggttcgtaaagctctgttgttagggaagaatgattgtatagtaactatatacagtagagacggtacctaaccagaaagccacggctaactacgtgccagcagccgcggtaatacgtaggtggcaagcgttgtccggaattattgggcgtaaagcgcgcgcaggtggtttaataagtctgatgtgaaagcccacggctcaaccgtggagggtcattggaaactgttaaacttgagtgcaggagagaaaagtggaattcctagtgtagcggtgaaatgcgtagagattaggaggaacaccagtggcgaaggcggctttttggcctgtaactgacactgaggcgcgaaagcgtggggagcaaacaggattagataccctggtagtccacgccgtaaacgatgagtgctaagtgttggtctcataagagatcagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaagtcttgacatactgtgaggacacaagagattgtgttgttctgacctttggttagacacagatacaggtggtgcatggttgtcgtcagctcgtgtcgtgagatgttgggttaagtcccgcaacgagcgcaacccctatgtctagttgccagcagtaagatggggactctagacagactgccagtgataaactggaggaaggcggggatgacgtcaaatcatcatgccccttatgacttgggctacacacgtgctacaatggataggaacaaagagaagcaaactcgcgagagcaagcaaacctcataaaactattctcagttcggattgtagtctgcaactcgactacatgaagctggaatcgctagtaatcgcgaatcagaatgtcgcggtgaatacgttcccgggtcttgtacacaccgcccgtcacaccacgagagtttgtaacacccgaagacggtggcctaacctttaaggagggagccggtcacggtgggacagatgattggggtgaagtcgtaacaaggtagccgtatcggaagg

SEQ ID NO: 11 - DNA, 1338 bp, Rothia aeria
aacacatgcaagtcgaacgatgaagcggtgcttgcacgtggattagtggcgaacgggtgagtaatacgtgagtgacctacctttgactctgggataagcctgggaaactgggtctaataccggatacgaccaatctctgcatggggtgttggtggaaagcgttatggagtggttttagatgggctcacggcctatcagcttgttggtgaggtaatggcttaccaaggcgacgacgggtagccggcctgagagggtgaccggccacactgggactgagacacggcccagactcctacgggaggcagcagtggggaatattgcacaatgggcgcaagctgatgcagcgacgccgcgtgagggatgacggccttcgggttgtaaacctctgttagcatcgaagaagcgaaagtgacggtaggtgcagagaaagcgccggctaactacgtgccagcagccgcggtaatacgtagggcgcgagcgttgtccggaattattgggcgtaaagagcttgtaggcggttggtcgcgtctgctgtgaaaggccggggcttaactccggttttgcagtgggtacgggctaactagagtgcagtaggggagactggaattcctggtgtagcggtggaatgcacagatatcaggaggaacaccgatggcgaaggcaggtctctgggctgtaactgacgctgagaagcgaaagcatggggagcgaacaggattagataccctggtagtccatgccgtaaacgttgggcactaggtgtgggggacattccacgttttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgcattagatcgcgtcagagatggcgttttccttcggggctggtgtacaggtggtgcatggttgtcgtcagctcgtgtcgtgagatgttgggttaagtcccgcaacgagcgcaaccctcgttctatgttgccagcggttcggccggggactcataggagactgccggggtcaactcggaggaaggtggggatgacgtcaaatcatcatgccccttatgtcttgggcttcacgcatgctacaatggccggtacagagggttgcgatactgtgaggtggagctaatcccttaaagccggtctcagttcggattggggtctgcaactcgaccccatgaagtcggagtcgctagtaatcgcagatcagcaacgctgcggtgaatacgttcccgggccttgtacacaccgcccgt

(19) United States
(12) Patent Application Publication
(10) Pub. No.: US 2023/0293599 A1
(43) Pub. Date: Sep. 21, 2023

(54) BACTERIAL COMPOSITIONS WITH ANTI-INFLAMMATORY ACTIVITY

(71) Applicant: Universiteit Gent, Gent (BE)

(72) Inventors: Aurélie Crabbe, Merelbeke (BE); Tom Coenye, Mariakerke (BE); Charlotte Rigauts, Ledeberg (BE)

(21) Appl. No.: 18/040,402

(22) PCT Filed: Jul. 29, 2021
(86) PCT No.: PCT/EP2021/071330

§ 371 Date: Feb. 2, 2023

(30) Foreign Application Priority Data
Aug. 3, 2020 (EP) 20189208.0
Oct. 6, 2020 (EP) 20200399.2

(51) Int. Cl. A61K 35/74; A61P 29/00; A61K 31/70

(52) U.S. Cl. CPC A61K 35/74; A61P 29/00; A61K 31/70

(57) ABSTRACT

The disclosure relates to bacterial compositions, prebiotics and methods of use thereof. The bacterial compositions may include bacteria of the genera Rothia, Gemella or Roseomonas, and combinations thereof. The bacterial compositions and prebiotics may be used in treating inflammation, such as inflammation of the respiratory tract and skin inflammation.

### CROSS-REFERENCE TO RELATED APPLICATIONS

This application is a national phase entry under 35 U.S.C. § 371 of International Patent Application PCT/EP2021/071330, filed Jul. 29, 2021, designating the United States of America and published as International Patent Publication WO 2022/029015 A2 on Feb. 10, 2022, which claims the benefit under Article 8 of the Patent Cooperation Treaty to European Patent Application Serial No. 20189208.0, filed Aug. 3, 2020, and to European Patent Application Serial No. 20200399.2, filed Oct. 6, 2020.

### STATEMENT REGARDING ELECTRONIC FILING OF A SEQUENCE LISTING

Pursuant to 37 C.F.R. § 1.821, a Sequence Listing ASCII text file entitled “3468-P17337US (P2020-038) Sequence Listing_ST25,” 22,843 bytes in size, generated Feb. 1, 2023, has been submitted via EFS-Web is provided in lieu of a paper copy. This Sequence Listing is hereby incorporated by reference into the specification for its disclosures.

### TECHNICAL FIELD

The disclosure relates to bacterial compositions and methods of use thereof. The bacterial compositions may include bacteria of the genera Rothia, Gemella or Roseomonas, and combinations thereof. The bacterial compositions may be used in treating inflammation, such as inflammation of the respiratory tract and skin inflammation.

### BACKGROUND

Chronic inflammation drives disease progression in respiratory disorders such as severe asthma, chronic obstructive pulmonary disease (COPD) and cystic fibrosis (CF). In these respiratory disorders, the pro-inflammatory pathway nuclear factor (NF)-kappaB (NE-κB) plays a central role as it mediates the response to a broad spectrum of inflammatory stimuli that include microorganisms, pollutants, or reactive oxygen species. NF-κB pathway activation in turn induces signaling systems that regulate cytokine activity, hereby contributing to lung pathology in chronic airway diseases. In CF, for example, the NE-κB pathway is involved in elevated levels of interleukin-8 (IL-8), which is the result of chronic infection and neutrophilic inflammation, and leads to progressive airway destruction. Moreover, the most effective current anti-inflammatory treatment for airway inflammation is corticosteroids that, among other pathways, inhibit the NT-κB pathway. Yet, ample side-effects and resistance issues have impeded their use in asthma, COPD and CF. Therefore, research into novel NF-κB pathway inhibitors may be beneficial for treating chronic airway diseases.

In the last decade, exploiting the microbiome to prevent and treat common health issues has gained immense interest. To this end, investigating the role of the gut microbiome in chronic inflammatory gut diseases such as Crohn's disease revealed commensals with therapeutic potential. For example, the gut commensal F. prausnitzii reduces gut inflammation through inhibition of the NF-κB pathway, making this bacterium a potential candidate for treatment of chronic gut inflammatory diseases that are related to microbiome dysregulation, such as ulcerative colitis and Crohn's disease (Cantin et al., 2015). Another example is the anti-inflammatory bacterium Lactobacillus rhamnosus that also exerts its probiotic activity through, among others, inhibition of the NE-κB pathway (Ind, 2005). While research on linking microbiome composition to disease parameters has mainly focused on the gut thus far, it has become clear that the severity, of chronic lung diseases such as asthma (Budden et al., 2019), COPD (Venkataraman et al., 2015) and CF (Venkataraman et al., 2015; Cope et al.) is influenced by lung microbiome composition and its associated inflammatory state (Yang et al., 2018; Beasley et al., 2012).

Inflammatory skin diseases are the most common problem in dermatology. They come in many forms, from occasional rashes accompanied by skin itching and redness, to chronic conditions such as dermatitis (eczema); acne vulgaris, rosacea, seborrheic dermatitis, and psoriasis. Interleukin-8 (IL-8) and IL-1 are potent chemotactic and pro-inflammatory cytokines, produced in the skin by a variety of cells in response to inflammatory stimuli. By far the most effective and commonly used prescription drugs for treating inflammation are corticosteroids, particularly the glucocorticoid related steroids.

Yet, current anti-inflammatory therapeutics, such as corticosteroids and NSAIDS, have a high risk of side effects and are, therefore, limited in use. Therefore, other approaches to reduce chronic inflammation are required.

### BRIEF SUMMARY

The disclosure relates to a method of preventing, reducing or treating inflammation in a subject, comprising administering a bacterial composition comprising or consisting of bacteria of the genera Rothia, Roseomonas or Gemella to the subject. In addition, the disclosure provides a method of preventing, reducing or treating (chronic) inflammation, in particular, respiratory or skin inflammation, in a subject, comprising administering a bacterial composition comprising or consisting of bacteria of the genera. Rothia, Gemella or Roseomonas to the subject. Furthermore, the disclosure provides combinations of bacteria, the combinations comprising or consisting of bacteria from at least two genera selected from the group consisting of: Rothia, Roseomonas and Gemella. In particular, the combination comprises or consists of at least two different bacterial species selected from the group consisting of: Rothia mucilaginosa, Rothia dentocariosa, Rothia terrae, Rothia amarae, Rothia aeria, Roseomonas gilardii, Roseomonas mucosa, Gemella haemolysans, Gemella bergeri, Gemella morbillorum and Gemella sanguinis; including derivatives or OTU encompassing the species. In a specific embodiment, the combination comprises or consists of the species Rothia mucilaginosa, Rothia dentocariosa and Roseomonas gilardii. Particular combinations comprise or consist of bacteria of and/or within the genera Rothia and Roseomonas. Even more particular, combinations comprise or consist of bacteria of and/or within the genera Rothia, Roseomonas and Gemella. In one embodiment, the combination is a bacterial composition or a bacterial consortium.

The disclosure also relates to the above bacteria, bacterial compositions or combinations and their use in therapy, more specifically, their use in preventing, reducing or treating inflammation or inflammatory conditions in a subject. The disclosure also provides these bacteria or combinations for use as a pro-biotic or dietary supplement.

In one embodiment, the subject is suffering from a respiratory disorder, in particular, lung inflammation, or from inflammation of the skin. In particular, the respiratory disorder is chronic. More in particular, the subject is diagnosed with or has a risk of developing cystic fibrosis (CF), chronic obstructive pulmonary disease (COPD), asthma, non-CF bronchiectasis, bronchitis, sarcoidosis, pneumonia, emphysema, inflammatory bowel disease, pulmonary fibrosis, sinusitis, mucositis, eczema, acne, rosacea, seborrheic dermatitis, or psoriasis.

In one embodiment, the bacteria or bacterial composition is formulated for local administration, such as administration to the respiratory tract of the subject or for nasal spray delivery, such as by nebulizer, inhalation, or aerosol. In another embodiment, the bacteria or bacterial composition is formulated for administration to the skin of the subject, such as by topical administration.

In one embodiment, the bacteria or bacterial composition is formulated as a dry powder or a liquid suspension.

In another embodiment, the administering results in an increase at the site of inflammation of at least one or more of bacterial populations of the genera Rothia, Roseomonas and/or Gemella in the subject. The increase may be at least 50%; more specifically, at least 100%, at least 150% or even more specifically, at least 200%.

In a further embodiment, the disclosure provides a pharmaceutical composition comprising or consisting of the bacteria as provided herein, and a pharmaceutically acceptable carrier and/or excipient.

In another embodiment, the disclosure provides a (pharmaceutical or prebiotic) composition comprising a compound that promotes the growth of the selected bacteria, wherein the compound is selected from the group consisting of: D,L-glycerol-phosphate, β-methyl-D-glucoside, D-trehalose, glycerol, maltose, maltotriose, N-acetyl-β-D-mannosamine, N-acetyl-D-glucosamine, sucrose, dextrin, oxomalic acid, salicin, and turanose, and combinations thereof. More specifically, the disclosure provides such a composition for use in preventing, reducing or treating inflammation in a subject, in particular, chronic or acute inflammatory conditions as specified herein, or inflammation of the skin. Furthermore, the disclosure provides a method of populating or increasing the microbiota comprising the genera Rothia, Roseomonas and/or Gemella, in the respiratory tract in a subject, comprising administering to the subject an effective amount of a compound selected from the group consisting of: D,L-glycerol-phosphate, β-methyl-D-glucoside, D-trehalose, glycerol, maltose, maltotriose, N-acetyl-β-D-mannosamine, N-acetyl-D-glucosamine, sucrose, dextrin, oxomalic acid, salicin, and turanose, and combinations thereof. Optionally, one or more of the compounds is administered in combination with the selected bacteria.

### BRIEF DESCRIPTION OF THE DRAWINGS

FIGS. 1A-1F: 3-D lung epithelial responses to pro-inflammatory- stimuli in the presence and absence of members of the lung microbiota. (FIG. 1A) IL-8 production by 3-D A549 cells after a 4-hour exposure to single bacterial cultures or to co-cultures of various lung microbiota members with P. aeruginosa PAO1 at MOI 30:1. (FIG. 1B) IL-8 production by 3-D A549 cells after a 24-hour exposure to 100 μg/mL LPS alone or in co-culture with R. mucilaginosa at an MOI 1:1. (FIG. 1C) IL-6, IL-8, GM-CSF and MCP-1 production of 3-D A549 cells after a 4-hour exposure to P. aeruginosa alone or in co-culture with R. mucilaginosa at MOI 10:1. (FIG. 1D) IL-8 production by 3-D bronchial epithelial CF cells (IB-3) or healthy (CFTR-corrected) 3-D bronchial epithelial cells (S9) after a 4-hour infection with P. aeruginosa PAO1 in combination with R. mucilaginosa at MOI 10:1 or (FIG. 1E) after a 24-hour exposure to 100 μg/mL LPS in combination with R. mucilaginosa at MOI 1:1. (FIG. 1F) IL-8 production by 3-D A549 cells after a 4-hour exposure to various pro-inflammatory stimuli (S. aureus, 100 μg/mL LPS, 100 μg/mL rhamnolipid (Rhl), 1 mM H₂O₂) alone or in co-culture with R. mucilaginosa at an MOI of 10:1. Data represent the mean IL-8 concentration (μg/mL) (FIGS. 1A, 1B, 1E, and 1F) or mean log cytokine concentration (μg/mL)±SEM (C), n≥3, *p<0.05, **p<0.001. NC=negative control, uninfected 3-D epithelial cells in serum-free medium; PAO1=P. aeruginosa PAO1; S=S. aureus SP123; St=S. anginosus LMG 14696; A=A. xylosoxidans LMG 26680; G=G. haemolysans LMG 18984; R=R. mucilaginosa DSM 20746.

FIGS. 2A and 2B: Influence of different R. mucilaginosa strains on the pro-inflammatory response of 3-D A549 cells to various P. aeruginosa strains and dosages. (FIG. 2A) IL-8 production by 3-D A549 cells after 4 hours exposure to single P. aeruginosa PAO1 culture at various MOI or to co-cultures of P. aeruginosa PAO1 with R. mucilaginosa DSM 20746 at MOI 10:1. (FIG. 2B) IL-8 production by 3-D A549 cells after a 4-hour exposure to various P. aeruginosa strains (PAO1, CF127, AA2, AA44, AA43) in single or co-cultures with various strains of R. mucilaginosa (DSM 20746, ATCC49042) at MOI 10:1. Data represent the mean IL-8 concentration (μg/mL)±SEM, n≥3, *p<0.05, **p<0.01

FIGS. 3A and 3B: Influence of R. mucilaginosa DSM20746 on the in vivo responses to LPS. MIP-2 concentration (measured by ELISA) (FIG. 3A), cytokine concentrations (measured by Bioplex) (FIG. 3B), and number of CFU/mL. Data represent the mean cytokine concentration (μg/mL) or mean log CFU/mL±SEM, n≥3, **p<0.01, ***p<0.001. Vehicle=sterile agar beads; LPS=10 μg/50 μL; Rothia=R. mucilaginosa DSM20746.

FIG. 4: Differential expression of genes involved in inflammation by 3-D A549 alveolar epithelial cells exposed to P. aeruginosa versus P. aeruginosa in combination with R. mucilaginosa. Quantitative RT-PCR analysis, showing fold changes in mRNA of 3-D A549 cells stimulated by a co-culture of P. aeruginosa and R. mucilaginosa versus mRNA of 3-D A549 cells stimulated only by P. aeruginosa. Data represent the fold change±SEM, n=3, *p<0.05.

FIGS. 5A-5D: Effect of R. mucilaginosa on NF-κB pathway activation by P. aeruginosa. (FIG. 5A) Activation of NF-κB pathway measured via luminescence of 3-D NF-κB reporter A549 cells. 3-D cells were exposed for 4 hours to P. aeruginosa PAO1 alone or in co-culture with R. mucilaginosa DSM 20746. (FIG. 5B) Semiquantitative determination (by Western blotting) of proteins (i.e., A20, IKB, p65 and P-IKB) produced by 3-D A549 cells stimulated with P. aeruginosa PAO1 with or without R. mucilaginosa DSM20746. (FIGS. 5C and 5D) Band intensity over time of Western Blot of 3-D A549 cells stimulated with P. aeruginosa PAO1 with or without R. mucilaginosa DSM20746. NC=negative control, uninfected 3-D NF-κB reporter A549 cells in serum-free GTSF-2 medium. Data represent the mean luminescence±SEM, n=3, *p<0.05, **p<0.01.

FIG. 6: Anti-inflammatory effect of various Rothia species. Quantification of NF-κB activation by P. aeruginosa PAO1 alone or in co-culture with various clinical Rothia species (measured by luminescence of a 3-D A549 reporter cell line). Data represent the mean luminescence±SEM, n≥3, *p<0.05 compared to P. aeruginosa PAO1.

FIG. 7: Minimal effective dose of R. mucilaginosa. Quantification of NF-κB activation by various MOI of P. aeruginosa PAO1 alone or in co-culture with various MOI of R. mucilaginosa DSM20746 (measured by luminescence of a 3-D A549 reporter cell line). PAO1=3-D A549 cells infected for 4 hours with P. aeruginosa PAO1; Rothia=3-D A549 cells infected for 4 hours with R. mucilaginosa DSM 20746; Data represent the mean luminescence±SEM, n≥3, *p<0.05, **p<0.01.

FIGS. 8A-8I: Screening of potential anti-inflammatory bacteria via NF-κB-luciferase reporter assay. Quantification of NF-κB activation by LPS in combination with various bacteria at varying MOI after 4 hours incubation. Data represent the mean luminescence±StDev, n≥3, *p<0.05.

FIG. 9: Reduction in NF-κB pathway activation by bacterial supernatant. Quantification of NF-κB activation by LPS in combination with various bacterial supernatants after a 4-hour incubation. Data represent the mean luminescence±StDev, n≥3, *p<0.05.

FIG. 10: Effect of the SCFA acetate on LPS-stimulated NF-κB pathway activation in 3-D A549 lung epithelial cells. Quantification of NF-κB activation by LPS in combination with various concentrations of acetate (i.e., 28.4 mM, 18.0 mM and 16.5 mM) after a 4-hour incubation. Data represent mean luminescence±StDev, n=3.

FIG. 11: Evaluating the anti-inflammatory effect of different species of the genus Gemella via NF-κB-luciferase reporter assay. Quantification of NF-κB activation by LPS in combination with bacteria at varying MOI after 4 hours incubation. Data represent the mean luminescence±StDev, n≥3, *p<0.05.

FIG. 12: Evaluating the anti-inflammatory effect of Roseomonas mucosa via NF-κB-luciferase reporter assay. Quantification of NF-κB activation by LPS in combination with bacteria at varying MOI after 4 hours incubation. Data represent the mean luminescence±StDev, n≥3, *p<0.05.

FIGS. 13A and 13B: Determination of minimal effective dose for reduction in NF-κB pathway activation. (FIG. 13A) The minimal effective MOI of each bacterium needed for reduction in LPS-induced NF-κB response was determined in single culture, in mixed culture of R. mucilaginosa, R. dentocariosa and R. gilardii (Mix 1), and in mixed culture of these bacteria with G. haemolysans (Mix 2). n/a: not applicable; FICI: fractional inhibitory concentration index (Stein et al., 2015). (FIG. 13B) The minimal effective MOI of each bacterium needed for reduction in LPS-induced NF-κB response in single culture, or in mixed culture of R. mucilaginosa and G. haemolysans haemolysans. Data represents mean % reduction in NF-κB pathway activation±StDev, n=3.

FIGS. 14A and 14B: (FIG. 14A) Determination of the minimal effective dose for reduction in NF-κB pathway activation induced by LPS, by R. mucilaginosa, R. dentocariosa and R. gilardii as single cultures, mixtures of two species or mixtures of three species. (FIG. 14B) Determination of the minimal effective dose for reduction in NF-κB pathway activation induced by LPS, by R. mucilaginosa, R. dentocariosa and R. terrae as single cultures, mixtures of two species or mixtures of three species, n/a: not applicable; FICI: fractional inhibitory concentration index (Stein et al., 2015).

FIG. 15: LDH release of HaCaT cells exposed to R. mucilaginosa at an MOI 10:1 with or without LPS for 4 hours and 24 hours, presented as %LDH compared to positive control (lysed cells with 1% TRITON® X-100). Error bars represent standard error. N≥3.

FIG. 16: LDH release of HaCaT cells exposed to R. dentocariosa at an MOI 10:1 with or without LPS for 4 hours, presented as %LDH compared to positive control (lysed cells with 1% TRITON® X-100). Error bars represent standard error. N≥3.

FIG. 17: LDH release of HaCaT cells exposed to R. mucilaginosa at an MOI 10:1 with or without P. acnes at an MOI of 10:1 for 48 hours, presented as %LDH compared to positive control (lysed cells with 1% TRITON® X-100). Error bars represent standard error. N≥3.

FIG. 18: LDH release of HaCaT cells exposed to R. dentocariosa at an MOI 10:1 with or without P. acnes at an MOI of 10:1 for 48 hours, presented as %LDH compared to positive control (lysed cells with 1% TRITON® X-100). Error bars represent standard error. N≥3.

FIG. 19: IL-8 concentration (μg/ml) produced by HaCaT keratinocytes in response to LPS stimulation, in the presence or absence of R. mucilaginosa at an MOI of 10:1 for 4 hours or 24 hours. Error bars represent standard error. N≥3, *p<0.05.

FIG. 20: IL-8 concentration (μg/ml) produced by HaCaT keratinocytes in response to P. acnes infection, in the presence or absence of R. mucilaginosa at an MOI of 10:1 for 48 hours. Error bars represent standard error. N≥3, *p<0.05.

FIG. 21: IL-8 concentration (μg/ml) produced by HaCaT keratinocytes in response to LPS stimulation, in the presence or absence of R. dentocariosa at an MOI of 10:1 for 4 hours. Error bars represent standard error. N≥3, *p<0.05.

FIG. 22: IL-8 concentration (μg/ml) produced by HaCaT keratinocytes in response to P. acnes infection, in the presence or absence of R. dentocariosa at an MOI of 10:1 for 48 hours. Error bars represent standard error. N≥3, *p<0.05.

FIGS. 23A-23F: 16S ribosomal RNA, partial sequences of the disclosed bacterial species.

### DETAILED DESCRIPTION

Unless indicated or defined otherwise, all terms used have their usual meaning in the art, which will be clear to the skilled person. As used herein, the singular forms “a,” “an,” and “the,” include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein. Unless otherwise indicated, the term “at least” preceding a series of elements is to be understood to refer to every element in the series. Those skilled in the art will recognize, or be able to ascertain using no more than routine experimentation, many equivalents to the specific embodiments of the disclosure described herein. Such equivalents are intended to be encompassed by the disclosure. The term “and/or” wherever used herein includes the meaning of “and,” “or” and “all or any other combination of the elements connected by the term.” The term “about” or “approximately” as used herein means within 20%, preferably within 15%, more preferably within 10%, and most preferably within 5% of a given value or range.

Throughout this specification and the claims that follow, unless the context requires otherwise, the word “comprise,” and variations such as “comprises” and “comprising,” will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having.”

In one embodiment, the disclosure provides bacteria, bacterial compositions and methods of use thereof. The bacteria and bacterial compositions may be used, without limitation to treat a subject for a condition characterized by inflammation in a specific organ or tissue. Hence, the bacteria and bacterial compositions may be used, to prevent, treat or reduce inflammation, in particular, to treat inflammatory disease or skin inflammation.

The disclosure has identified bacterial genera each having anti-inflammatory properties, i.e., the genera Rothia, Gemella and Roseomonas. More specifically, it was demonstrated for the first time that several bacterial species within the genera are able to inhibit the major pro-inflammatory pathway NF-κB (nuclear factor kappa-light-chain-enhancer of activated B cells), which is currently a target for anti-inflammatory drugs. The NF-κB pathway regulates cytokine activity and is found to be chronically active in many inflammatory diseases, such as chronic airway diseases, inflammatory bowel disease, arthritis, gastritis, atherosclerosis and others. The bacterial composition of the disclosure may include one or more bacterium (in some embodiments, also referred to as a bacterial population or bacterial culture), of the genus Rothia, Gemella or Roseomonas. In one embodiment, a bacterial composition comprises or consists of bacteria of at least two genera selected from Rothia, Gemella or Roseomonas. In a further embodiment, a bacterial composition comprises or consists of bacteria of the three genera Rothia, Gemella and Roseomonas. In one composition different species within one genus can be present. Typically, members of these genera can be identified by 16s rRNA sequencing analysis or MALDI-TOF, or any other method generally known to the skilled person. More specifically, the composition can further include a carrier and/or excipient, as specified hereinbelow.

In particular embodiments, a (bacterial) composition as described herein may consist of or include the following combinations of bacteria of the genera:
Rothia and Roseomonas,
Rothia and Gemella,
Roseomonas and Gemella, or
Rothia, Roseomonas and Gemella.

It was demonstrated in the disclosure that within each of these genera bacteria possess anti-inflammatory properties, and that combinations or consortia of bacteria show a synergistic effect therein, hence combining different species of the bacteria can be an advantage. In general, bacterial genera are designated based on phenotypic features common to the members of the genus so that one of skill in the art can “visualize or recognize” the members of the genus and/or on genome-based taxonomy of the genus using methods known the skilled person (e.g., by use of Buchanan, R. E. and Gibbons, N. E. (1974) Bergey's Manual of Determinative Bacteriology, 8th Edition, Williams and Wilkins, Baltimore, 1268 p.), or as described herein.

In a particular embodiment, bacteria of the genus Rothia include the bacterial strains Rothia mucilaginosa, Rothia dentocariosa, Rothia terrae, Rothia amarae, and Rothia aeria; or operational taxonomic unit (OTU) encompassing the species. In one embodiment, a Rothia mucilaginosa strain (previously known as Stomatococcus mucilaginosus) comprises the 16S rRNA gene set forth under SEQ ID NO:1 (genome: GenBank accession number GCA_000175615.1). As provided herein, a bacterial strain of Rothia mucilaginosa is characterized by a 16S rRNA gene sequence having at least 95%, 96%, 97%, 98% or 99% sequence identity to the 16S rRNA gene set forth under SEQ ID NO:1, or wherein the bacterial strain has a 16S rRNA gene sequence represented by SEQ ID NO:1. Exemplary strains are the Rothia mucilaginosa strain DSM20746 deposited at DSMZ (16S ribosomal RNA, partial sequence NCBI Reference Sequence: NR_044873.1), or strain ATCC49042 deposited at the American Type Culture Collection; or a derivative thereof.

In a further embodiment, a Rothia dentocariosa strain comprises the 16S rRNA gene set forth under SEQ ID NO:2 (genome: GenBank accession number GCA_000164695.2). As provided herein, a bacterial strain of Rothia dentocariosa is characterized by a 16S rRNA gene sequence having at least 95%, 96%, 97%, 98% or 99% sequence identity to the 16S rRNA gene set forth under SEQ ID NO:2, or wherein the bacterial strain has a 16s rRNA gene sequence represented by SEQ ID NO:2. An exemplary strain is Rothia dentocariosa strain HVQC18-02 deposited at BCCM under accession no. LMG 31869, or strain CDC X599 deposited at ATCC (no. 17931; 16S ribosomal RNA, partial sequence NCBI Reference Sequence: NR_074568.1); or a derivative thereof.

In a further embodiment, a Rothia terrae strain comprises the 16S rRNA gene set forth under SEQ ID NO:3 (genome: GenBank accession number GCA_012396615.1). As provided herein, a bacterial strain of Rothia terrae is characterized by a 16S rRNA gene having at least 95%, 96%, 97%, 98% or 99% sequence identity to the 16S rRNA gene set forth under SEQ ID NO:3, or wherein the bacterial strain has a 16s rRNA gene sequence represented by SEQ ID NO:3. An exemplary strain is the Rothia terrae strain deposited at BCCM/LMG under number 23708 (16S ribosomal RNA, partial sequence NCBI Reference Sequence: NR_043968.1); or a derivative thereof.

In a further embodiment, a Rothia amarae strain comprises the 16S rRNA gene set forth under SEQ ID NO:4 (genome: GenBank accession number AY043359.1). As provided herein, a bacterial strain of Rothia amarae is characterized by a 16S rRNA gene having at least 95%, 96%, 97%, 98% or 99% sequence identity to the 16S rRNA gene set forth under SEQ ID NO:4. In a further embodiment, a Rothia amarae strain comprises the 16S rRNA gene set forth under SEQ ID NO:4, or wherein the bacterial strain has a 16s rRNA gene sequence represented by SEQ ID NO:4. An exemplary strain is the Rothia amarae strain deposited under No. 47294T at CCUG (no. 47294; 16S ribosomal RNA, partial sequence NCBI Reference Sequence: NR_029045.1); or a derivative thereof.

In a further embodiment, a Rothia aeria strain comprises the 16S rRNA gene set forth under SEQ ID NO:11 (genome: GenBank accession number: NR_024785.1). As provided herein, a bacterial strain of Rothia aeria is characterized by a 16S rRNA gene having at least 95%, 96%, 97%, 98% or 99% sequence identity to the 16S rRNA gene set forth under SEQ ID NO:11. In a further embodiment, a Rothia amarae strain comprises the 16S rRNA gene set forth under SEQ ID NO:11, or wherein the bacterial strain has a 16s rRNA gene sequence represented by SEQ ID NO:11. An exemplary strain is the Rothia aeria strain A1-17B (16S ribosomal RNA, partial sequence NCBI Reference Sequence: NR_024785.1); or a derivative thereof.

As indicated herein, an anti-inflammatory effect was demonstrated for different species and strains of Rothia, and hence is not limited to a specific Rothia species or strain. In addition, it can be of interest to combine two, three, four or more different Rothia species in the compositions or methods as provided herein. Hence, the disclosure provides a bacterial composition comprising bacteria of 2, 3, 4, or 5 of the species selected from the group consisting of: Rothia mucilaginosa, Rothia dentocariosa, Rothia terrae, Rothia amarae, and Rothia aeria. The disclosure also provides the composition for use in the methods as disclosed herein.

In a particular embodiment, bacteria of the genus Roseomonas may include Roseomonas gilardii or Roseomonas mucosa, or operational taxonomic unit (OTU) encompassing the species. In one embodiment, a bacterium of the genus Roseomonas is the Roseomonas sp. Roseomonas gilardii. In a further embodiment, a Roseomonas gilardii strain comprises the 16S rRNA gene set forth under SEQ ID NO:5 (genome: Bioproject number PRJNA234800). As provided herein, a bacterial strain of Roseomonas gilardii is characterized by a 16S rRNA gene having at least 95%, 96%, 97%, 98% or 99% sequence identity to the 16S rRNA gene set forth under SEQ ID NO:5. An exemplary strain is the Roseomonas gilardii deposited at ATCC under number 49956 (16S ribosomal RNA, partial sequence NCBI Reference Sequence: NR_029061.1), or a derivative thereof.

In a further embodiment, a Roseomonas mucosa strain comprises the 16S rRNA gene set forth under SEQ ID NO:6 (genome: GenBank accession number GCA_000622225.1). As provided herein, a bacterial strain of Roseomonas mucosa is characterized by a 16S rRNA gene having at least 95%, 96%, 97%, 98% or 99% sequence identity to the 16S rRNA gene set forth under SEQ ID NO:6. An exemplary strain is the Roseomonas mucosa deposited at ATCC under number BAA-692 (16S ribosomal RNA, partial sequence NCBI Reference Sequence: NR_028857.1), or a derivative thereof.

As indicated herein, an anti-inflammatory effect was demonstrated for different species and strains of Roseomonas, and hence is not limited to a specific Roseomonas species or strain. In addition, it can be of interest to combine the two disclosed Roseomonas species in the compositions or methods as provided herein.

In a particular embodiment, bacteria of the genus Gemella may include Gemella haemolysans, G. asaccharolytic, G. bergeri, G. cuniculi, G. morbillorum, G. palaticanis, G. parahaemolysans, G. sanguinis, or G. taiwanensis; or operational taxonomic unit (OTU) encompassing the species, and, in particular, includes Gemella haemolysans, G. bergeri, G. morbillorum and G. sanguinis. In one embodiment, the bacterium of the genus Gemella is the Gemella sp. Gemella haemolysans. In a further embodiment, a Gemella haemolysans strain comprises the 16S rRNA gene set forth under SEQ ID NO:7 (genome: GenBank accession number GCA_000173915.1). As provided herein, a bacterial strain of Gemella haemolysans is characterized by a 16S rRNA gene having at least 95%, 96%, 97%, 98% or 99% sequence identity to the 16S rRNA gene set forth under SEQ ID NO:7. An exemplary strain is the Gemella haemolysans deposited at ATCC under number 10379 (16S ribosomal RNA, partial sequence NCBI Reference Sequence: NR_025903.1), or a derivative thereof.

In a further embodiment, a Gemella bergeri strain comprises the 16S rRNA gene set forth under SEQ ID NO:8 (genome: GenBank accession number GCA_000469465.1). As provided herein, a bacterial strain of Gemella bergeri is characterized by a 16S rRNA gene having at least 95%, 96%, 97%, 98% or 99% sequence identity to the 16S rRNA gene set forth under SEQ ID NO:8. An exemplary strain is the Gemella bergeri strain 617-93 deposited at ATCC under number 700627 (16S ribosomal RNA, partial sequence NCBI Reference Sequence: NR_026420.1), or a derivative thereof.

In a further embodiment, a Gemella morbillorum strain comprises the 16S rRNA gene set forth under SEQ ID NO:9 (Bioproject number PRJNA33175). As provided herein, a bacterial strain of Gemella morbillorum is characterized by a 16S rRNA gene having at least 95%, 96%, 97%, 98% or 99% sequence identity to the 16S rRNA gene set forth under SEQ ID NO:9. An exemplary strain is the Gemella morbillorum strain 2917B deposited at ATCC under number 27824 (16S ribosomal RNA, partial sequence NCBI Reference Sequence: NR_025904.1), or a derivative thereof.

In a further embodiment, a Gemella sanguinis strain comprises the 16S rRNA gene set forth under SEQ ID NQ:10 (genome: Bioproject number PRJNA33175). As provided herein, a bacterial strain of Gemella sanguinis is characterized by a 16S rRNA gene having at least 95%, 96%, 97%, 98% or 99% sequence identity to the 16S rRNA gene set forth under SEQ ID NQ:10. An exemplary strain is the Gemella sanguinis strain 2045-94 deposited at ATCC under number 700632 (16S ribosomal RNA, partial sequence NCBI Reference Sequence: NR_026419.1), or a derivative thereof.

In one embodiment, a Gemella asaccharolytic strain is WAL 1945J(T) (=ATCC BAA-1630(T); CCUG 57045(T)), a derivative thereof or OTU encompassing the species; a G. parahaemolysans strain is NTUH_1465(T) (=BCRC 80365(T); JCM 18067(T)), a derivative thereof or OTU encompassing the species; a Gemella palaticanis strain is CCUG 39489T (ATCC BAA-58), a derivative thereof or OTU encompassing the species; a Gemella cuniculi strain is CCUG 42726T (A TCC BAA-287), a derivative thereof or OTU encompassing the species; and a Gemella taiwanensis strain is NTUH_5572(T) (=BCRC 80366(T); JCM 18066(T)), a derivative thereof or OTU encompassing the species. As indicated herein, an anti-inflammatory effect was demonstrated for different species and strains of Gemella, hence it can be of interest to combine two, three, four or more of the disclosed Gemella species in the compositions or methods as provided herein.

In one embodiment, the (bacterial) composition of the disclosure consists of or comprises the following combinations of bacteria, including OTU encompassing the given species and/or derivatives thereof:
Bacteria within the genus Rothia, in particular, Rothia mucilaginosa, Rothia dentocariosa, Rothia terrae, Rothia amarae, or Rothia aeria;
Bacteria within the genus Roseomonas, in particular, Roseomonas gilardii or Roseomonas mucosa;
Bacteria within the genus Gemella, in particular, Gemella haemolysans, Gemella asaccharolytic, Gemella bergeri, Gemella cuniculi, Gemella morbillorum, Gemella palaticanis, Gemella parahaemolysans, Gemella sanguinis, or Gemella taiwanensis; more in particular, Gemella haemolysans, G. bergeri, G. morbillorum or G. sanguinis;
Bacteria of the genus Rothia, in particular, Rothia mucilaginosa, Rothia dentocariosa, Rothia terrae, Rothia amarae, or Rothia aeria, and bacteria of the genus Roseomonas, in particular, Roseomonas gilardii or Roseomonas mucosa;
Bacteria of the genus Rothia, in particular, Rothia mucilaginosa, Rothia dentocariosa, Rothia terrae, Rothia amarae, or Rothia aeria, and bacteria of the genus Gemella, in particular, Gemella haemolysans, Gemella asaccharolytic, Gemella bergeri, Gemella cuniculi, Gemella morbillorum, Gemella palaticanis, Gemella parahaemolysans, Gemella sanguinis, or Gemella taiwanensis; more in particular, G. haemolysans, G. bergeri, G. morbillorum or G. sanguinis;
Bacteria of the genus Roseomonas, in particular, Roseomonas gilardii or Roseomonas mucosa, and bacteria of the genus Gemella, in particular, Gemella haemolysans, Gemella asaccharolytic, Gemella bergeri, Gemella cuniculi, Gemella morbillorum, Gemella palaticanis, Gemella parahaemolysans, Gemella sanguinis, or Gemella taiwanensis; more in particular, G. haemolysans, G. bergeri, G. morbillorum or G. sanguinis;
Bacteria of the genus Rothia, in particular, Rothia mucilaginosa, Rothia dentocariosa, Rothia terrae, Rothia amarae, or Rothia aeria, bacteria of the genus Roseomonas, in particular, Roseomonas gilardii or Roseomonas mucosa, and bacteria of the genus Gemella, in particular, Gemella haemolysans, Gemella asaccharolytic, Gemella bergeri, Gemella cuniculi, Gemella morbillorum, Gemella palaticanis, Gemella parahaemolysans, Gemella sanguinis, or Gemella taiwanensis; more in particular, G. haemolysans, G. bergeri, G. morbillorum or G. sanguinis.

Combinations can comprise 2, 3, 4, 5, 6, 7, 8, 9, 10, 15, or more different bacterial species, in particular, 2-10, more in particular, 2, 3, 4 or 5.

Specific combinations comprise or consist of the following bacterial species, including OTU encompassing the species and/or derivatives thereof:
Rothia mucilaginosa and Rothia dentocariosa;
Rothia mucilaginosa and Roseomonas gilardii;
Rothia dentocariosa and Roseomonas gilardii;
Rothia mucilaginosa and Gemella haemolysans;
Rothia dentocariosa and Gemella haemolysans;
Roseomonas gilardii and Gemella haemolysans;
Rothia mucilaginosa, Rothia dentocariosa and Roseomonas gilardii;
Rothia mucilaginosa, Rothia dentocariosa, Roseomonas gilardii and Gemella haemolysans;
Rothia mucilaginosa, Rothia dentocariosa, Rothia terrae;
Rothia mucilaginosa and Rothia terrae; or
Rothia dentocariosa and Rothia terrae.

In a further embodiment, the disclosure provides a bacterial composition, the composition comprising bacteria of at least two, in particular three, of the genera Rothia, Roseomonas or Gemella, wherein the ratio of Rothia to Roseomonas to Gemella is ranging from 1:1:4 to 1:40:40, in particular, from 1:1:8 to 1:32:32. The ratios can be applied to the herein mentioned combinations or compositions, including those comprising the specifically mentioned bacterial species, but can differ according to the application as can be determined by the skilled person.

In another embodiment, the bacterial composition comprises bacteria of at least two, in particular, at least three, species within the genera Rothia, Roseomonas and/or Gemella. Specific ratios can, for example, be as follows:

| Rothia | Rothia | Roseomonas |
|---|---|---|
| mucilaginosa | dentocariosa | gilardii |
| 1 | 1 | 8 |
| 1 | 2 | 8 |
| 1 | 4 | 8 |
| 1 | 8 | 8 |
| 1 | 16 | 8 |
| 1 | 1 | 16 |
| 1 | 2 | 16 |
| 1 | 4 | 16 |
| 1 | 8 | 16 |
| 1 | 16 | 16 |
| 1 | 8 | 32 |
| 1 | 16 | 32 |
| 1 | 32 | 32 |

| Rothia | Rothia | Rothia | Roseomonas | Rothia | Roseomonas |
|---|---|---|---|---|---|
| mucilaginosa | dentocariosa | mucilaginosa | (e.g., R. gilardii) | dentocariosa | (e.g., R. gilardii) |
| 1 | 1 | 1 | 8 | 1 | 1 |
| 1 | 2 | 1 | 16 | 1 | 2 |
| 1 | 4 | 1 | 32 | 1 | 4 |
| 1 | 8 | | | 1 | 8 |
| 1 | 16 | | | 1 | 16 |
| 1 | 32 | | | 2 | 1 |

The term “derivatives” as used herein refers to bacteria that are derived by means of serial passage from the original or parent strain as a starting material and that preferably have the same anti-inflammatory activity. By “operational taxonomic unit” or “OTU” is meant classification of microbes within the same, or different, OTUs using techniques known in the art. OTU refers to a terminal leaf in a phylogenetic tree and is defined by a nucleic acid sequence, e.g., the entire genome, or a specific genetic sequence, and all sequences that share sequence identity to this nucleic acid sequence at the level of species. In some embodiments, the specific genetic sequence may be the 16S rRNA sequence of a bacterium, or a portion of the 16S rRNA sequence. In other embodiments, the entire genomes of two organisms can be sequenced and compared. In another embodiment, select regions such as multilocus sequence tags (MLST), specific genes, or sets of genes may be genetically compared. In 16S rRNA embodiments, OTUs that share >97% average nucleotide identity across the entire 16S rRNA or some variable region of the 16S rRNA are considered the same OTU. In embodiments involving the complete genome, MLSTs, specific genes, or sets of genes OTUs that share >95% average nucleotide identity are considered the same OTU. OTUs are in some cases defined by comparing sequences between organisms. Generally, sequences with less than 95% sequence identity are not considered to form part of the same OTU. OTUs may also be characterized by any combination of nucleotide markers or genes, in particular, highly conserved genes (e.g., “house-keeping” genes), or a combination thereof. Such characterization employs, e.g., WGS data or a whole genome sequence. “16S sequencing” or “16S-rRNA” or “16S” refers to sequence derived by characterizing the nucleotides that comprise the 16S ribosomal RNA gene(s). The bacterial 16S rDNA is approximately 1500 nucleotides in length and is used in reconstructing the evolutionary relationships and sequence similarity of one bacterial isolate to another using phylogenetic approaches. 16S sequences are used for phylogenetic reconstruction as they are in general highly conserved, but contain specific hypervariable regions that harbor sufficient nucleotide diversity to differentiate genera and species of most bacteria, as well as fungi. In some, embodiments, OTUs may be determined using CrunchClust (Hartmann et al., Significant and persistent impact of timber harvesting on soil microbial communities in Northern coniferous forests, The ISME Journal 6, 2199-2218 (2012)) and classified against the Greengenes Database (DeSantis, T. Z. et al., Greengenes, a Chimera-Checked 16S rRNA Gene Database and Workbench Compatible with ARB, Appl. Environ. Microbiol. 72: 5069-5072 (2006)) according to 97% similarity.

In one embodiment, a bacterial composition as described herein may include bacteria comprising a 16S rRNA gene sequence substantially identical to the sequence set forth in SEQ ID NOs. 1 to 11. By “substantially identical” is meant a nucleic acid sequence that differs from a reference sequence only by one or more conservative substitutions, or by one or more non-conservative substitutions, deletions, or insertions located at positions of the sequence that do not destroy the biological function of the nucleic acid molecule. Such a sequence can be any integer at least 70%, 75%, 80%, 85%, 90% or over 95%, or more generally at least 95%, 96%, 97%, 98%, 99%, or 100% identical when optimally aligned at the nucleotide level to the sequence used for comparison using, for example, FASTA. For nucleic acid molecules, the length of comparison sequences may be at least 5, 10, 15, 20, or 25 nucleotides, or at least 30, 40, or 50 nucleotides. In alternate embodiments, the length of comparison sequences may be at least 60, 70, 80, or 90 nucleotides, or over 100, 200, or 500 nucleotides, or, in particular, over the full length sequence as given in the SEQ ID NOs. Sequence identity can be readily measured using publicly available sequence analysis software (e.g., BLAST software available from the National Library of Medicine; e.g., NCBI Blast v2.0, using standard settings). Such software matches similar sequences by assigning degrees of homology to various substitutions, deletions, substitutions, and other modifications.

The selected bacteria can be obtained by any suitable manner known in the art. For example, the bacteria may be isolated from a natural environment or purchased from a suitable commercial source such as, e.g., the American Type Culture Collection (ATCC) (10801 University Boulevard, Manassas, VA 20110 USA), the German Collection of Microorganisms and Cell Cultures (DSMZ), Leibniz Institute, Germany or LM-UGent (BCCM/LMG Bacteria Collection, Karel Lodewijk Ledeganckstraat 35, 9000 Gent, Belgium). In one embodiment, the bacteria as described herein are present in the lungs, oral cavity or sputum from a donor or individual, in particular, a healthy donor or individual. Such bacteria may be “directly isolated” and not resulting from any culturing or other process that results in or is intended to result in replication of the population after obtaining the material. In a further embodiment, bacteria as described herein include bacterial spores. The bacterial strains for use in the disclosure can be cultured using standard microbiology techniques, e.g., as detailed in the present examples. In particular, the bacteria are “live bacteria” of the genera or species as identified herein. Also, a composition as described herein may include substantially pure bacteria of the genera or species as defined herein. By “substantially pure” or “isolated” is meant bacteria of the genera Rothia, Roseomonas or Gemella, including the specified species that are separated from the components that naturally accompany it. Typically, a bacterial composition as described herein is substantially pure when it is at least 50%, 60%, 70%, 75%, 80%, 85%, 90%, 9%, 92%, 93%, 94%, 95%, 96%, 97%, 98% or 99% by weight, of the total material in a sample or formulation. A substantially pure bacterial composition, as described herein, can be obtained, for example, by extraction from a natural source, such as the lungs or oral cavity, from an individual (e.g., according to the procedures as provided in the examples section), or from bacterial cultures, for example, cultures of any of the bacteria described herein, e.g., commercially available.

The herein identified isolated bacteria of the disclosure, or the derivatives thereof, may be in the form of live bacteria, dead bacteria or cellular components, fermentation broths, cell culture supernatants (either liquid, concentrated or dried), cell free lysates derived from culture media of these bacteria, or extracts of bacteria, fermentation broths, or supernatants. In particular, the bacteria are isolated, purified and viable. In one embodiment, the bacterial composition of the disclosure comprises a bacterial population, a fermentation broth or supernatant thereof, of the bacteria of the disclosure, i.e., of bacteria of the genus Rothia, Roseomonas and/or Gemella, including the species as identified herein. In a particular embodiment, the disclosure provides a combination of two or more strains of Rothia mucilaginosa, Rothia dentocariosa, Rothia terrae, Rothia amarae, Rothia aeria, Roseomonas gilardii, Roseomonas mucosa, Gemella haemolysans, Gemella bergeri, Gemella morbillorum or Gemella sanguinis; and/or one or more culture supernatant or cell free lysates derived from culture media in which one or more of the strains has been cultured for the uses and methods as provided herein. As used herein, the term “supernatant” refers to the liquid remaining when cells that are grown in broth or harvested in another liquid from an agar plate are removed by centrifugation, filtration, sedimentation, or other means well known in the art. It was demonstrated in the disclosure that, e.g., R. mucilaginosa and R. dentocariosa supernatant had significant anti-inflammatory properties. Hence in one embodiment, the bacterial composition comprises R. mucilaginosa or R. dentocariosa supernatant, or a mixture thereof. The composition is of particular interest for use in the methods as defined herein. In another embodiment, the bacterial composition comprises bacteria of the genus Rothia and/or Roseomonas and a supernatant from bacteria of the genus Rothia, in particular, R. mucilaginosa and/or R. dentocariosa supernatant.

### Use

The disclosure provides the bacteria or bacterial composition as disclosed herein before for use as a medicament. In general, the bacteria and bacterial composition of the disclosure can be used to inhibit NF-κB pathway activation and/or the production of inflammatory cytokines such as, e.g., MCP-1, IL-6, IL-8, IL-lalpha, IL-5, IL-10 and GM-CSF, in particular, IL-8. Nuclear factor (NF)-kappaB (NF-κB) is a common transcriptional regulator of inflammation, in particular, of or associated with respiratory diseases and skin disorders. As used herein, “inflammation” is part of the complex biological response of body tissues to harmful stimuli, such as pathogens, damaged cells, radiation or irritants, and is a protective response involving immune cells, blood vessels, and molecular mediators. The function of inflammation is to eliminate the initial cause of cell injury, clear out necrotic cells and tissues damaged from the original insult and the inflammatory process, and initiate tissue repair. The five classical signs of inflammation are heat, pain, redness, swelling, and loss of function.

It was demonstrated in the disclosure that several bacterial species from the genus Rothia have anti-inflammatory properties. Results obtained in the present study indicate that the presence of R. mucilaginosa in the airways could be beneficial as it did not only inhibit the P. aeruginosa-induced IL-8 response, it also showed an inhibitory effect on the IL-8 response induced by LPS, oxidative stress and by S. aureus, another important respiratory pathogen. Surprisingly, the anti-inflammatory effect was demonstrated for a large collection of Rothia species and strains, indicating this effect is not species- or strain-specific. More specifically, at least five different Rothia species from various sources were found to similarly inhibit inflammation. As such, in one embodiment, the disclosure provides the bacterial species Rothia dentocariosa, Rothia terrae, Rothia amarae, and/or Rothia aeria for use in the methods as provided herein, and, in particular, for use in the reduction or treatment of inflammation. Also within the genus Roseomonas, more than one species was identified having anti-inflammatory properties, such as Roseomonas gilardii and Roseomonas mucosa. Likewise, an anti-inflammatory effect was demonstrated for several Gemella species, such as Gemella haemolysans, Gemella morbillorum and Gemella sanguinis.

Individual bacterial species can be used as well as combinations thereof. Moreover, it was for the first time demonstrated herein that bacteria from the genera Roseomonas and Gemella have anti-inflammatory properties, in particular, the bacterial species Roseomonas gilardii, Roseomonas mucosa, Gemella haemolysans, Gemella morbillorum and Gemella sanguinis. As shown in the present examples, LPS-stimulated NF-κB pathway activation was inhibited by G. haemolysans and R. gilardii. In one embodiment, the disclosure provides the bacterial species Roseomonas gilardii, Roseomonas mucosa, Gemella haemolysans, Gemella morbillorum and/or Gemella sanguinis (individually or in combination) for use in the methods as provided herein, and, in particular, for use in the reduction or treatment of inflammation.

R. mucilaginosa was also able to reduce the production of the pro-inflammatory cytokines IL-6, GM-CSF, IL-1β,l chemokine MCP-1 at the protein and/or mRNA level to background concentrations in vitro, which was also confirmed in an in vivo model. Lung homogenates of mice following 48 hours co-exposure to LPS and Rothia-embedded agar beads contained significantly lower levels of cytokines MIP-2, MCP-1, IL-6, IL-1α, IL-5, TNF-α and GM-CSF than those of mice exposed to LPS alone. R. mucilaginosa caused a clear decline in the inflammatory response of lung tissue regardless of the stimulus. In addition, it was demonstrated that absolute abundance of R. mucilaginosa was negatively correlated with inflammatory markers (based on IL-8 and IL-1(3 levels) and mediators of airway remodeling (MMP-1 and MMP-8 levels). Levels of MMP-1, MMP-8, and MMP-9, which degrade extracellular matrix proteins (Vandenbroucke et al., 2011), are increased in the airways of patients with neutrophilic airways disease, are inversely correlated with lung function, and contribute to irreversible airway damage. Hence, it is of particular interest to include R. mucilaginosa in a composition of the disclosure. In addition, R. mucilaginosa and R. dentocariosa were able to inhibit the production of the pro-inflammatory cytokine IL-8 in keratinocytes. Keratinocytes constitute 90% of the cells of the epidermis, the outermost layer of the skin. The primary function of keratinocytes is the formation of a barrier against environmental damage by heat, UV radiation, water loss, pathogenic bacteria, fungi, parasites, and viruses. Pathogens invading the upper layers of the epidermis can cause keratinocytes to produce proinflammatory mediators, particularly chemokines such as IL-8, CXCL10 and CCL2 (MCP-1), which attract neutrophils, monocytes, natural killer cells, T-lymphocytes, and dendritic cells to the site of pathogen invasion. For example, Propionibacterium acnes (Cutibacterium acnes) induces the production of IL-8 in keratinocytes, leading to the recruitment of neutrophils to the pilosebaceous unit, in turn contributing to inflammation and development of acne. IL-8 production induced by P. acnes is among others regulated by the NF-κB pathway. Keratinocytes also modulate the immune system: apart from the above-mentioned antimicrobial peptides and chemokines they are also potent producers of anti-inflammatory mediators such as IL-10 and TGF-β. When activated, they can stimulate cutaneous inflammation and Langerhans cell activation via TNFα and IL-1β secretion. In one embodiment, the disclosure provides the bacterial species R. mucilaginosa and R. dentocariosa (individually or in combination) for use in the methods as provided herein, and, in particular, for use in the reduction or treatment of skin inflammation. In view of their anti-inflammatory properties, other Rothia species can be used for the application, as well as species of the genus Roseomonas and Gemella, as identified herein.

Hence in one embodiment, the bacteria or bacterial composition, as described herein, may be used in a method to prevent, reduce or treat inflammation in a subject. More specifically, the disclosure provides the bacteria or bacterial composition for use in preventing, treating or reducing inflammation, in particular, for preventing, reducing or treating (chronic or acute) inflammatory disease such as chronic airway disease, skin inflammation (e.g., acne, eczema (or atopic dermatitis), rosacea, seborrheic dermatitis, or psoriasis), inflammatory bowel disease and mucositis (e.g., radio mucositis). In one embodiment, inflammation may be provoked by an external stimulus such as, e.g., a pathogen, allergens, (cigarette) smoke, tissue damage, radiation, etc., and involves a mechanism of the innate immune system. “Chronic” inflammation also known as prolonged inflammation, leads to a progressive shift in the type of cells present at the site of inflammation, such as mononuclear cells, and is characterized by simultaneous destruction and healing of the tissue from the inflammatory process. Whereas, “acute” inflammation is the initial response of the body to harmful stimuli and is achieved by the increased movement of plasma and leukocytes (especially granulocytes) from the blood into the injured tissues.

In a specific embodiment, the bacteria and bacterial compositions as identified herein can be used to treat (lung) inflammation associated with chronic respiratory diseases, such as asthma, chronic obstructive pulmonary disease (COPD), cystic fibrosis (CF), bronchiectasis, bronchitis, (chronic) sinusitis, sarcoidosis, pneumonia, emphysema or lung fibrosis. In a further embodiment, the bacteria and bacterial compositions as identified herein can be used to treat acute inflammation associated with respiratory diseases, such as bronchitis or sinusitis caused by bacterial or viral infection. Acute bronchitis is normally caused by a viral infection, typically rhinovirus, parainfluenza, or influenza. A small number of cases are due to bacteria such as Mycoplasma pneumoniae or Bordetella pertussis. Acute sinusitis is usually preceded by an earlier upper respiratory tract infection, generally of viral origin, mostly caused by rhinoviruses, coronaviruses, and influenza viruses, others caused by adenoviruses, human parainfluenza viruses, human respiratory syncytial virus, enteroviruses other than rhinoviruses, and metapneumovirus. If the infection is of bacterial origin, the most common three causative agents are Streptococcus pneumoniae, Haemophilus influenzae, and Moraxella catarrhalis. In another embodiment, the bacteria and bacterial compositions as identified herein can be used to treat skin inflammation or inflammatory conditions of the skin, such as acne, eczema (or atopic dermatitis), rosacea, seborrheic dermatitis, and psoriasis. In one embodiment, the disclosure provides bacteria from the genus Rothia for treating acne vulgaris (such as, e.g., at least in part induced by Propionibacterium acnes) and, in particular, the bacterial species Rothia mucilaginosa or Rothia dentocariosa within these genera. In view of their anti-inflammatory properties, other Rothia species can be used for the application, as well as species of the genus Roseomonas and Gemella, as identified herein.

In a further aspect, the bacteria and compositions of the disclosure can be used to treat non-responder patient populations having an inflammatory disease as indicated herein. More specifically, the patients no longer responded to the standard therapies, which rely on corticosteroids as anti-inflammatories, and hence, fail to control symptoms in a significant number of patients; for example, corticosteroid resistance is a problem in COPD as well as asthma.

Therefore, in one aspect, the disclosure provides a method of treatment and/or prevention of inflammatory diseases of the respiratory system including rebalancing of the immune system and/or normalization of the NF-κB pathway, the method comprising administration of the bacteria or bacterial composition as provided herein to a subject. As used herein “inflammatory disease of the respiratory system” includes asthma, bronchiectasis, bronchitis, sinusitis, COPD, sarcoidosis, pneumonia, emphysema, lung fibrosis, and cystic fibrosis.

In a further embodiment, the bacteria or bacterial composition, as described herein, may be used in a method to alter the microbiota of the respiratory tract, in particular, the lower respiratory tract, more in particular, the lungs. In an even further embodiment, the bacteria or bacterial composition, as described herein, may be used in a method to populate the respiratory tract, in particular, the lower respiratory tract, more in particular, the lungs. By “populating the respiratory tract” is meant establishing a healthy state of the microbiota or microbiome in a subject. In some embodiments, populating the respiratory tract includes increasing the levels of specific bacteria in the respiratory tract. By “altering the microbiota of the respiratory tract” is meant any change, either increase or decrease, of the microbiota or microbiome in a subject. In some embodiments, altering the microbiota of the respiratory tract includes increasing the levels of specific bacteria described herein. The “respiratory tract” as used herein includes the upper respiratory tract, i.e., the parts of the respiratory system lying above the sternal angle (outside of the thorax), and consisting of the nasal cavity and paranasal sinuses, and the pharynx (nasopharynx, oropharynx and laryngopharynx); and the lower respiratory tract, also called the respiratory tree or tracheobronchial tree, and referring to the branching structure of airways supplying air to the lungs, and includes the trachea, bronchi, bronchioles and lungs (including alveoli).

By “increase,” “increasing,” “decrease” or “decreasing” is meant a change in the levels of specific bacteria, e.g., in the respiratory tract of a subject. An increase or decrease may include a change of any value between 30% and 500%, for example, a change of about 30%, 50%, 70%, 90%, 100%, 150%, 200%, or more, when compared to a control. In some embodiments, the increase or decrease may be a change of about or at least 2-fold, 3-fold, 4-fold, 5-fold, 10-fold, 50-fold, 100-fold, or more, when compared to a (non-treated) control.

“Microbiota” refers to the community of microorganisms that occur (sustainably or transiently) in and on a subject, typically a mammal such as a human. “Microbiome” refers to the genetic content of the communities of microbes that live in and on the human body, both sustainably and transiently, including eukaryotes, archaea, bacteria, and viruses (including bacterial viruses, such as phage), where “genetic content” includes genomic DNA, RNA such as ribosomal RNA, the epigenome, plasmids, and other types of genetic information.

The terms “treatment,” “treating” or “therapy” encompass prophylactic, palliative, therapeutic, and nutritional modalities of administration of the bacteria or bacterial compositions described herein. Accordingly, treatment includes amelioration, alleviation, reversal, or complete elimination of one or more of the symptoms of inflammation in a subject diagnosed with, or known to have, a (chronic or acute) inflammatory condition, such as, e.g., provided hereinbefore, or be considered to derive benefit from the alteration of microbiota at the site of inflammation, such as, e.g., the (lower) respiratory tract or the skin. In one embodiment, treatment includes reduction of inflammation as measured by one or more of the following: decreased levels of pro-inflammatory cytokines or increased levels of anti-inflammatory cytokines in sputum or bronchoalveolar lavage fluid, by at least 5%, 10%, 20% 30%, 40%, 50%, 60%, 70%, 80%, 90%, 95% or more; reduced infiltration of immune cells (such as neutrophils) in the lung environment (based on cell counts of bronchoalveolar lavage fluid or sputum), improvement of lung function as measured through forced expiratory volume in one second (FEV1) or forced vital capacity (FVC), reduced number of exacerbations and increased time to next exacerbation; less or reduced clinical symptoms of skin inflammation including the presence of inflammatory lesions and comedones. Treatment also includes prevention or delay of the onset of inflammation, e.g., associated with respiratory disease or skin inflammation.

As used herein, a subject may be a mammal, such as a human, non-human primate (e.g., monkey, baboon, or chimpanzee), rat, mouse, rabbit, cow, horse, pig, dog, cat, etc. In some embodiments, the subject is a human or human patient. In some embodiments, the subject may have undergone, be undergoing, or about to undergo, antibiotic and/or corticosteroids therapy. The subject may be a clinical patient, a clinical trial volunteer, an experimental animal, etc. The subject may be suspected of having or at risk for chronic disease; or be diagnosed with chronic or acute disease, such as, e.g., the diseases as disclosed herein. Diagnostic methods for chronic or acute disease, and the clinical delineation of such diagnoses are known to those of ordinary skill in the art. In some embodiments, the subject may be an individual considered to be benefitted by the alteration of microbiota. In some embodiments, the subject may be an individual considered to be benefitted by population of the respiratory tract or of the skin.

### Pharmaceutical Compositions, Dosages and Administration

The bacteria or bacterial compositions can be employed therapeutically in compositions formulated for administration by any conventional route, e.g., intranasal, by inhalation, by aerosol, topical, dermal, via gavage, rectal or by oral administration. In one embodiment, the delivery is local such as to the site of inflammation.

In one embodiment, the administration of at least one bacterium or bacterial composition, as provided herein, is intranasal (e.g., as a powder, nasal drop or aerosol). In a specific embodiment, the bacteria or bacterial compositions are delivered to the subject by airway administration or intrapulmonary administration. As used herein the terms “intrapulmonary, intratracheal, intrabronchial or intra alveolar administration” include all forms of such administration whereby a bacterium or bacterial composition is applied into the trachea, the bronchi or the alveoli, respectively, whether by instillation of a solution comprising the bacteria or bacterial composition, by applying the bacteria or bacterial composition in a powder form, or by allowing the bacteria or bacterial composition to reach the relevant part of the airway by inhalation as an aerosolized or nebulized solution or suspension or inhaled powder or gel, with or without added stabilizers or other excipients. Methods of airway or intrapulmonary administration include, but are not limited to aerosols according to methods well known to those skilled in the art, comprising the bacteria or the bacterial composition; or by any other effective form of intrabronchial administration including the use of inhaled powders containing the bacteria or bacterial composition in dry form, with or without excipients, or the direct application of the bacteria or bacterial composition, in solution or suspension or powder form during bronchoscopy. Other administration methods are tracheal washing, inhalation of nebulized fluid droplets the instillation or application of a solution of bacteria or bacterial composition or a powder or a gel containing the bacteria or bacterial composition into the trachea or lower airways. Preferred methods of administration may include using a nebulizer, a metered dose inhaler (MDI) or a dry powder inhaler system (DPI).

In another embodiment, the administration of at least one bacterium or bacterial composition, as disclosed herein, is topical. Hence, the bacteria or bacterial composition as described above is therefore used as an active ingredient in the preparation of a formulation for dermatological use, optional in combination with a dermatologically acceptable excipient. The definition “dermatologically acceptable” as used herein indicates that the excipient is suitable for application on human skin without toxicity risk, incompatibility, instability, allergic response, and the like.

In a further embodiment, the administration is oral, subcutaneous or intravenous.

The (bacterial) compositions can be in a variety of forms. These forms include, e.g., liquid, semi-solid and solid dosage forms. The bacteria or bacterial compositions, as described herein, can be formulated as a pharmaceutical composition and include a pharmaceutically acceptable carrier and/or excipient. Pharmaceutically acceptable carriers and/or excipients are familiar to those skilled in the art. As used herein, a “pharmaceutically acceptable carrier” refers to, and includes, any and all solvents, dispersion media, liposomal or nanoparticle preparation, coatings, antibacterial and antifungal agents, isotonic and absorption delaying agents, and the like that are physiologically compatible. The compositions can include a pharmaceutically acceptable salt, e.g., an acid addition salt or a base addition salt. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

In a particular embodiment, the composition of the disclosure comprises an excipient, carrier and/or adjuvant and at least 50%, more specifically at least 60%, even more specifically at least 70%, 80% or 90% of single or combinations of the live bacteria, dead bacteria or cellular components, fermentation broths, cell culture supernatants (either liquid, concentrated or dried), cell free lysates derived from culture media of these bacteria, or extracts of bacteria, fermentation broths, or supernatants as provided herein.

The compositions may be sterilized by conventional techniques well known to those skilled in the art. The resulting aqueous solutions may be packaged for use or filtered under aseptic conditions and freeze-dried, the freeze-dried preparation being dissolved in a sterile aqueous solution prior to administration.

The compositions may contain pharmaceutically acceptable auxiliary substances or adjuvants, including, without limitation, pH adjusting and buffering agents and/or tonicity adjusting agents, such as, for example, sodium acetate, sodium lactate, sodium chloride, potassium chloride, calcium chloride, etc. The bacteria or bacterial compositions, as described herein, can be provided alone or in combination with other compounds or compositions, in the presence of a carrier, in a form suitable for administration to a subject.

The herein disclosed bacteria or bacterial compositions may be formulated in a wide variety of formulations for oral administration. Solid form preparations may include powders, tablets, drops, capsules, cachets, lozenges, and dispersible granules. Other forms suitable for oral administration may include liquid form preparations including emulsions, syrups, elixirs, aqueous solutions, aqueous suspensions, toothpaste, gel dentifrice, chewing gum, or solid form preparations that are intended to be converted shortly before use to liquid form preparations, such as solutions, suspensions, and emulsions.

In a further aspect of the disclosure, bacteria or the bacterial composition may be formulated for topical delivery or application, for example, as a liquid, emulsion, suspension, ointment, cream, gel, lotion, powder, or spray formulations. In certain embodiments, upon application to a skin of a subject, the composition or dosage form may form a patch. The topical dosage form may also include a pharmaceutically acceptable carrier. Suitable carriers that may be useful in topical formulations include, but are not limited to, solubilizers such as C2-C8, straight and branched chain alcohols, diols and triols, moisturizers and humectants such as glycerin, amino acids and amino acid derivatives, polyaminoacids and derivatives, pyrrolidone carboxylic acids and its salts and derivatives, surfactants such as sodium lauryl sulfate, sorbitan monolaurate, emulsifiers such as cetyl alcohol, stearyl alcohol, thickeners such as methyl cellulose, ethyl cellulose, hydroxymethylcellulose, hydroxypropylcellulose, polyvinylpyrollidone, polyvinyl alcohol and acrylic polymers. The composition or dosage form may be applied to the skin by any means known in the art including, for example, manually, by an aerosol spray, pump-pack, brush, swab, or other applicator. Various additives, known to those skilled in the art, may be included in the topical dosage forms.

Thus, the disclosure encompasses a pharmaceutical composition for use in a method of reducing inflammation in a subject, comprising administering to the subject the pharmaceutical composition, wherein the pharmaceutical composition comprises an isolated, anti-inflammatory bacterial population, such that inflammation in the subject is reduced, wherein the anti-inflammatory bacterial population comprises one or more bacterial species from the genus Rothia, Roseomonas and/or Gemella (e.g., the bacterial species or combinations as specified herein); in particular, wherein the pharmaceutical composition comprises or consists of a carrier, excipient and/or adjuvant and bacteria of the genus Rothia, Roseomonas and/or Gemella, even more in particular, of the genus Roseomonas and/or Gemella, or of the genus Gemella. In the embodiment, the level of the anti-inflammatory bacteria is augmented at the site of inflammation, such as, e.g., in the respiratory tract or on the skin of the subject. It furthermore has been shown in the disclosure that administration of combinations of bacteria selected from these genera or species can be of particular advantage since less bacteria are needed to obtain the same effect.

The bacteria or bacterial composition of the disclosure can be used alone, or in combination therapies with one, two, or more other pharmaceutical compounds or drug substances. In some embodiments, the bacteria or bacterial composition as described herein may be administered to a subject prior to, during, or subsequent to treatment with an antibiotic.

In one embodiment, the bacteria or the bacterial composition, as described herein, may be a therapeutic, prophylactic, nutritional or probiotic composition. Probiotics are one or more, or a mixture of, (live) microorganisms that, when administered in adequate amounts, confer a health benefit on the host. More specifically, the therapeutic, prophylactic, nutritional or probiotic composition includes the bacteria of the genera Rothia, Roseomonas and/or Gemella. Even more specifically, the composition may be a therapeutic, prophylactic, nutritional or probiotic composition including a bacterium from the genus Rothia (e.g., Rothia mucilaginosa, Rothia dentocariosa, Rothia terrae, Rothia amarae or Rothia aeria), a bacterium from the genus Roseomonas (e.g., Roseomonas gilardii or Roseomonas mucosa) or a bacterium from the genus Gemella (e.g., Gemella haemolysans, Gemella asaccharolytic, Gemella bergeri, Gemella cuniculi, Gemella morbillorum, Gemella palaticanis, Gemella parahaemolysans, Gemella sanguinis, or Gemella taiwanensis; in particular, Gemella haemolysans Gemella haemolysans, Gemella bergeri, Gemella morbillorum or Gemella sanguinis; or a combination thereof comprising at least two, in particular, at least three, four or five different species. Exemplary combinations are disclosed hereinbefore.

The bacteria or bacterial compositions can be provided once or twice; chronically, in a continuous mode for a certain period of time; or intermittently, with interruptions or in cycles. Combinations of bacteria may be administered simultaneously (e.g., as part of the same composition), or separately, e.g., successively.

Typically, the bacteria or bacterial composition is administered in an effective amount, such as, e.g., an amount sufficient to colonize the respiratory tract of a subject for a suitable period of time. In one embodiment, an effective amount includes a therapeutically effective amount or a prophylactically effective amount. A “therapeutically effective amount” refers to an amount effective, at dosages and for periods of time necessary, to achieve the desired therapeutic result, such as treatment, reduction, or amelioration of inflammation. A therapeutically effective amount of a (bacterial) composition may vary according to factors such as the disease state, age, sex, and weight of the subject, and the ability of the (bacterial) composition to elicit a desired response in the individual. Dosage regimens may be adjusted to provide the optimum therapeutic response. A “prophylactically effective amount” refers to an amount effective, at dosages and for periods of time necessary, to achieve the desired prophylactic result. Typically, a prophylactic dose is used in subjects prior to or at an earlier stage of disease or disease symptoms, so that a prophylactically effective amount may be less than a therapeutically effective amount.

A “probiotic” amount refers to an amount effective, at dosages and for periods of time necessary, to achieve the desired result, such as population of the respiratory tract of a subject after, for example, antibiotic treatment, to normal levels. Typically, probiotic doses are administered at large excess and may be significantly higher than prophylactically effective or therapeutically effective amounts.

A suitable range for therapeutically or prophylactically effective amounts, or probiotic amounts, of bacteria or a bacterial composition, as described herein, will be determined by the skilled person, and may include without limitation at least or about 10⁰, 10¹, 10², 10³, 10⁴, 10⁵, 10⁶, 10⁷, 10⁸, 10⁹, 10¹⁰, 10¹¹, 10¹², 10¹³, or 10¹⁴ colony forming units (cfus) of the bacteria, per unit dosage, in particular, between 10² and 10¹⁰ cfus per unit dose (the amount of a medication administered to a patient in a single dose).

In some embodiments, dosages for live bacteria, in vegetative or spore forms, can be about 1 μg to about 1000 mg, such as about 0.5 mg to about 5 mg, about 1 mg to about 1000 mg, about 2 mg to about 200 mg, about 2 mg to about 100 mg, about 2 mg to about 50 mg, about 4 mg to about 25 mg, about 5 mg to about 20 mg, about 10 mg to about 15 mg, about 50 mg to about 200 mg, about 200 mg to about 1000 mg, or about 1, 2, 3, 4, 5 or more than g per dose or composition; or 0.001 mg to 1 mg, 0.5 mg to 5 mg, 1 mg to 1000 mg, 2 mg to 200 mg, or 2 mg to 100 mg, or 2 mg to 50 mg, or 4 mg to 25 mg, or 5 mg to 20 mg, or 10 mg to 15 mg, or 50 mg to 200 mg, or 200 mg to 1000 mg, or 1, 2, 3, 4, 5 or more than 5 g per dose or composition. Dosage values may vary with the severity of the condition to be alleviated. For any particular subject, specific dosage regimens may be adjusted over time according to the individual need and the professional judgement of the person administering or supervising the administration of the compositions. For example, a single bolus may be administered, several divided doses maybe administered over time or the dose may be proportionally reduced or increased as indicated by the exigencies of the situation. The bacteria or composition of the disclosure may be administered daily or more frequently, such as twice or more per day.

### Prebiotics

In a further embodiment, the disclosure provides the use of compounds, in particular, carbon sources, to induce or promote the growth of the anti-inflammatory bacteria described herein (including R. mucilaginosa, R. dentocariosa, R. gilardii, and G. haemolysans) without inducing the growth of the common pathogens Pseudomonas aeruginosa and/or Staphylococcus aureus. Carbon sources for which the area under the curve (AUC) values were lower than 50, were considered as “no growth inducers.” D,L-glycerol-phosphate was demonstrated to enable growth of anti-inflammatory bacteria (i.e., R. mucilaginosa and R. dentocariosa but not of R. gilardii and G. haemolysans), while not or only minimally supporting growth of both S. aureus and P. aeruginosa. Twelve carbon sources did not support growth of one of both tested pathogens, i.e., P. aeruginosa (i.e., β-methyl-D-glucoside, D-trehalose, glycerol, maltose, maltotriose, N-acetyl-β-D-mannosamine, N-acetyl-D-glucosamine, sucrose, dextrin, oxomalic acid, salicin, and turanose). Of these twelve carbon sources, six (i.e., β-methyl-D-glucoside, D-trehalose, glycerol, maltose, maltotriose, and sucrose) allowed growth of both R. mucilaginosa and R. dentocariosa, three (i.e., dextrin, salicin, and turanose) stimulated only the growth of R. dentocariosa, one (oxomalic acid) enabled growth of both R. dentocariosa and R. gilardii, and two (i.e., N-acetyl-β-D-mannosamine and N-acetyl-D-glucosamine) supported growth of G. haemolysans. Hence, the disclosure provides the use of these compounds (as a “prebiotic”), individually or as a mixture, to stimulate the growth of the resp. bacteria in a subject in order to reduce or to treat inflammation in the methods and for the applications provided herein. Furthermore, the disclosure relates to a compound selected from the group consisting of: D,L-glycerol-phosphate, β-methyl-D-glucoside, D-trehalose, glycerol, maltose, maltotriose, N-acetyl-β-D-mannosamine, N-acetyl-D-glucosamine, sucrose, dextrin, oxomalic acid, salicin, and turanose, and mixtures thereof, for use in preventing, treating or reducing inflammation. One or more of these compounds may be used in combination with the bacteria, bacterial composition or pharmaceutical composition as described herein before, sometimes also referred to as “synbiotics” (a combination of prebiotics and probiotics).

In one embodiment, the bacterial composition of the disclosure can be combined with, comprises or consists of the following:
D,L-glycerol-phosphate and R. mucilaginosa and/or R. dentocariosa;
β-methyl-D-glucoside, D-trehalose, glycerol, maltose, maltotriose, and/or sucrose and R. mucilaginosa and/or R. dentocariosa;
dextrin, salicin, and/or turanose and R. dentocariosa;
oxomalic acid and R. dentocariosa and/or R. gilardii; or
N-acetyl-β-D-mannosamine and/or N-acetyl-D-glucosamine and G. haemolysans.

A sample may be analyzed to detect the levels of the anti-inflammatory bacteria by the detection methods disclosed herein further.

### Detection Methods

Also provided herein are methods of determining the likelihood if treatment with the bacteria of the disclosure will be effective in a patient by determining the levels of one or more bacteria of the genera Rothia, Roseomonas and/or Gemella in the subject. If the level is decreased it can be an advantage to apply the treatment as herein described before.

Also, the efficacy of the treatment may be monitored by determining the levels of one or more bacteria of the genera Rothia, Roseomonas and/or Gemella, or of bacteria-specific produced volatile compounds (VOCs), in a sample from the subject, and comparing the determined levels to previous determinations from the subject.

By “determining” or “detecting” it is intended to include determining the presence or absence of a substance or quantifying the amount of a substance, such as one or more of the bacteria described herein, or a metabolite as described herein. The term thus refers to the use of the materials, compositions, and methods described herein or known in the art for qualitative and quantitative determinations. An increase or decrease may include a change of any value between 30% and 500%, or more, when compared to a control. In some embodiments, the increase or decrease may be a change of about 2-fold, 3-fold, 4-fold, 5-fold, 10-fold, 100-fold, or more, when compared to a control. A “sample” can be any organ, tissue, body fluid, cell, or cell extract isolated from a subject, such as a sample isolated from a mammal, in particular, a human having, suspected of having, or having an inflammatory condition, such as, e.g., inflammation of the (lower) respiratory tract or the skin. For example, a sample can include, without limitation, cheek mucosa, saliva/sputum, lung tissue, blood, or any other specimen obtained from a patient (human or animal), or test subject. A “control” includes a sample obtained for use in determining base-line expression or activity. Accordingly, a control sample may be obtained from a healthy individual, such as an individual not suffering from inflammation, in particular, of the lower respiratory tract or inflammation of the skin. A control also includes a previously established standard or reference, e.g., as used in the present examples. Accordingly, any test or assay may be compared with the established standard and it may not be necessary to obtain a control sample for comparison each time. The sample may be analyzed to detect the presence or levels of a Rothia, Roseomonas and/or Gemella gene (including a species-specific gene), genome, peptide, protein, nucleic acid molecule, such as a Rothia, Roseomonas and/or Gemella 16S rRNA molecule, using methods that are known in the art, such as quantitative PCR; or of bacteria-specific produced volatile compounds (VOCs). Analysis can also be done by culturing/isolation on specific selective media followed by enumeration of colony forming units. Or by staining with specific probes (that target proteins, nucleic acids) and detection using microscopic or flow cytometry analysis. Or by detection of species-specific metabolites or volatile metabolites using GC-MS, SIFT-MS.

The following examples are set forth below to illustrate the methods, compositions, and results according to the disclosed subject matter. These examples are not intended to be inclusive of all aspects of the subject matter disclosed herein, but rather to illustrate representative methods, compositions, and results. These examples are not intended to exclude equivalents and variations of the disclosure, which are apparent to one skilled in the art.

### EXAMPLES

### Example 1: Anti-Inflammatory Properties of Bacteria of the Genus Rothia

### MATERIALS AND METHODS

### Bacterial Species and Culturing Conditions

Six bacterial species (Table 1) commonly isolated from the CF lung were selected for this study. These include two pathogens commonly isolated from persons with CF (P. aeruginosa and S. aureus), two less frequently recovered CF pathogens (Streptococcus anginosus and Achromobacter xylosoxidans) and two bacteria that are not commonly considered as CF pathogens, but that can often be isolated from CF lower airway secretions (Rothia mucilaginosa and Gemella haemolysans). For select experiments, additional Rothia species, i.e., R. dentocariosa, R. terrae, R. amarae and R. aeria, isolated from the oral cavity, sputum or the environment were also included. Except for the species obtained from culture collections, all isolates were characterized by MALDI-TOF (Cattoir, L., et al. 2018) and the Bioyper reference database version 4.1.80, Bruker Daltonics Inc., Billerica, MA. All isolates were cultured at 37° C. under constant shaking conditions (250 rpm) in Brain Heart Infusion (BHI) broth (Lab M, Lancashire, UK) until stationary phase. Most isolates were cultured in aerobic conditions except for S. anginosus and G. haemolysans, which were cultured in microaerobic conditions (<1% O₂) (Campygen Compact system, Oxoid, Thermo Fischer Scientific).

TABLE 1
Overview of strains used in this study

| | Strain | |
|---|---|---|
| Species | designation | Source |
| Pseudomonas | PA01 (ATCC | Wound |
| aeruginosa | 15692) | |
| | AA2 | Early CF isolate (De Soyza et al., 2013) |
| | AA44 | Late CF isolate (De Soyza et al., 2013) |
| | AA43 | Late CF isolate (De Soyza et al., 2013) |
| | CF127 | CF isolate, hyper-biofilm former |
| | | (Colvin et al., 2012) |
| Staphylococcus | SP123 | Sputum of mechanically-ventilated |
| aureus | | patient (Vandecandelaere et al., 2012) |
| Streptococcus | LMG14696 | Human throat |
| anginosus | | |
| Achromobacter | LMG26680 | Sputum of CF patient |
| xylosoxidans | | |
| Gemella | LMG18984 | Unknown |
| haemolysans | | |
| Rothia | DSM20746 | Throat |
| mucilaginosa | ATCC49042 | Bronchial secretion |
| | HVOC02-02 | Oral cavity healthy volunteer |
| | HVOC02-03 | Oral cavity healthy volunteer |
| | HVOCO3-01 | Oral cavity healthy volunteer |
| | HVOC03-02 | Oral cavity healthy volunteer |
| | HVOC15-01 | Oral cavity healthy volunteer |
| | HVOC23-01 | Oral cavity healthy volunteer |
| | HVOC24-01 | Oral cavity healthy volunteer |
| | R-36507 | Terrestrial microbial mat |
| | | (Antarctica) (Peeters et al. 2012) |
| Rothia | HVOC01-01 | Oral cavity healthy volunteer |
| dentocariosa | HVOC05-01 | Oral cavity healthy volunteer |
| | HVOC10-01 | Oral cavity healthy volunteer |
| | HVOC17-01 | Oral cavity healthy volunteer |
| | HVOC26-01 | Oral cavity healthy volunteer |
| | HVOC27-01 | Oral cavity healthy volunteer |
| | HVOC28-01 | Oral cavity healthy volunteer |
| Rothia terrae | HVOC29-01 | Oral cavity healthy volunteer |
| Rothia amarae | R-43211 | Terrestrial microbial mat |
| | | (Antarctica) (Peeters et al. 2012) |
| | R-37581 | Terrestrial microbial mat |
| | | (Antarctica) (Peeters et al. 2012) |
| | R38387 | Terrestrial microbial mat |
| | | (Antarctica) (Peeters et al. 2012) |
| | R36663 | Terrestrial microbial mat |
| | | (Antarctica) (Peeters et al. 2012) |
| Rothia aeria | HVOC04-01 | Oral cavity healthy volunteer |
| | HVOC12-01 | Oral cavity healthy volunteer |
| | HVOC13-01 | Oral cavity healthy volunteer |
| | HVOC14-01 | Oral cavity healthy volunteer |

### 3-D Lung Epithelial Cell Culture Models

A549 cell line. A previously developed organotypic three-dimensional (3-D) cell culture model was used to study the immune response to bacterial infection and various pro-inflammatory stimuli (i.e., LPS, rhamnolipid, H₂O₂) (Barrila et al., 2010; Crabbkkket al., 2016; Carterson et al., 2005; Crabbé et al., 2011). Three-dimensional (3-D) in vivo-like lung epithelial cell culture model systems reflect key aspects of the parental tissue, including 3-D architecture, barrier function, apical-basolateral polarity, and multicellular complexity (Crabbé et al., 2008; Barrila et al., 2010; Crabbé et al., 2016). It has been demonstrated that P. aeruginosa adhesion, as well as host-secreted cytokine profiles, in 3-D cell culture models of lung epithelial cells are more similar to the in vivo situation than cells grown as a monolayer on plastic (Carterson et al., 2005). In short, the human adenocarcinoma alveolar lung epithelial cell line A549 (ATCC CCL-185) was first grown as a monolayer in T75 cell culture flasks containing GTSF-2 medium (HyClone, Logan, UT) supplemented with 1.5 g/L sodium bicarbonate (Sigma-Aldrich), 10% fetal bovine serum (FBS, Life technologies), 2.5 mg/L insulin transferring sodium selenite (Lonza) and 1% penicillin-streptomycin (Life Technologies) at 37° C. under 5% CO₂. At confluence, the cells were seeded in the Rotating Wall Vessel (RWV) with type-1 collagen coated dextran beads (Cytodex-3 microcarrier beads, Sigma) at a cell to bead ratio of 2:1. For all infection studies, the 3-D A549 cells were cultured in the RWV bioreactor for 11 to 14 days. The 3-D aggregates were transferred to 48-well plates at a concentration of 2.5×10⁵ cells/well containing fresh serum-free GTSF-2 medium on the day of the infection studies.

IB-3 and S9 cell lines. The above-mentioned 3-D cell culture model was optimized for use with the IB-3 and S9 cell lines. The IB-3 cell line is a bronchial epithelial cell line heterogeneous for F508del (F508del/W1282X). The S9 cell line originates from the IB-3 cell line and is stably transduced with wild-type Cystic Fibrosis Transmembrane Conductance Regulator (CFTR). LHC-8 medium without gentamicin (Life Technologies) supplemented with 5% FBS and 1% penicillin-streptomycin was used to culture both cell lines. The 3-D models of IB-3 and S9 cells were generated as described for the 3-D A549 model, except that a cell to bead ratio of 4:1 was used. Upon maturation, the cells were transferred to 48-well plates (2.5×10⁵ cells/well) containing fresh serum-free medium on the day of the infection studies.

In vitro infection studies. Prior to infection, bacterial cultures were centrifuged and resuspended in host cell culture medium, i.e., serum-free GTSF-2 (HyClone, Logan, UT) supplemented with 1.5 g/L sodium bicarbonate (Sigma-Aldrich) and 2.5 mg/L insulin transferring sodium selenite (Lonza) or LHC-8. 3-D cell aggregates containing 2.5×10⁵ cells were seeded into 48-well plates filled with fresh medium. Subsequently, cells were infected with single cultures of P. aeruginosa, S. aureus, S. anginosus, A. xylosoxidans, G. haemolysans or R. mucilaginosa for 4 hours at a multiplicity of infection (MOI) of 10:1 (=10 bacteria per lung epithelial cell), unless indicated otherwise. Additionally, cells were infected with P. aeruginosa together with each one of the above-mentioned CF lung microbiota members for 4 hours at a 10:1 ratio, resulting in an MOI of 20:1. When indicated, 3-D cell aggregates were exposed to P. aeruginosa or other pro-inflammatory stimuli, i.e., S. aureus (MOI 10:1), lipopolysaccharide (LPS) (10011g/mL) (Sigma-Aldrich), rhamnolipid (100 μg/mL) (Sigma-Aldrich) and H₂O₂ (100 mM), together with R. mucilaginosa for 4 hours. For longer term experiments, 3-D cell aggregates were exposed to 100 μg/mL LPS alone or in combination with R. mucilaginosa for 24 hours at an MOI of 1:1.

Cytotoxicity assays. After infection of the 3-D cells with various bacteria or exposure to other pro-inflammatory stimuli (LPS, rhamnolipid and H₂O₂), cell viability was assessed using the following two methods, (i) AnnexinV/PI assay. After infection, cells were washed with Hank's Balanced Salt Solution (HBSS, Life Technologies) and aggregates were dissociated into individual cells by treatment with 0.25% trypsin-EDTA (Life Technologies). Assessment of epithelial cell viability, differentiating between live, apoptotic and necrotic cell populations, was performed using the Annexin V-propidium iodide (PI) kit for flow cytometry analysis according to the manufacturer's instructions. In short, dissociated cells were harvested by centrifugation (6 minutes, 1,200 rpm) and washed with PBS. After re-centrifugation, cells were resuspended in 100 μL 1× Annexin-binding buffer supplemented with 5 μL Alexa Fluor 488 AnnexinV and 2 μL of 100 μg/mL PL After a 15 minutes incubation period, this mixture was diluted 1:5 in IX Annexin-binding buffer and the fluorescence was analyzed by an Attune NxT flow cytometer (Thermo Fischer Scientific) at 530 nm and 575 nm using 488 nm excitation, (ii) Lactate dehydrogenase (LDH) assay. After infection, cell culture supernatant was centrifuged (6 minutes, 1,200 rpm) and supernatant was used for detection of LDH released from epithelial cells in co-culture with bacteria. LDH levels were measured using the LDH detection kit (Sigma-Aldrich) according to the manufacturer's instructions. In short, 5 μL of supernatant was diluted in 45 μL LDH buffer. Next, 50 μL LDH master mix was added to each well and absorbance at 450 nm was measured each 5 minutes. LDH activity was calculated using an NADH standard curve.

Bacterial cell adherence analysis. Bacterial adherence to the host cells was determined as described previously (Anderson et al., 2008; Crabbé et al., 2017; Tran et al., 2014). Briefly, 3-D epithelial cell aggregates were transferred to a new 48-well plate and washed twice with HBSS. Bacterial cells were dissociated from epithelial cells using 0.1% TRITON® X-100 and then quantified by plating on previously developed selective media: Luria Bertani (LB) agar supplemented with 1.25 mg/mL triclosan to select for P. aeruginosa, nutrient agar supplemented with 5 mg/mL mupirocin and 10 mg/mL colistin sulphate to select for R. mucilaginosa, and LB agar supplemented with 7.5% NaCl to select for S. aureus (Vandeplassche et al, 2017).

In vivo mouse model. Twelve-week old female BALB/c ByJRj mice (Janvier Labs) were managed in accordance to the guidelines provided by the European Directive for Laboratory Animal Care (Directive 2010/63/EU of the European Parliament). The laboratory Animal Ethics Committee of the University of Antwerp authorized and approved all animal experiments in this study (file 2019-90). To prepare the inoculum for intratracheal infection, R. mucilaginosa cryostock was thawed, incubated in 50 mL falcons with TSB broth for 18 hours at 180 rpm and 37° C. The overnight culture was centrifuged at 4,750 rpm. The supernatant was discarded, and 1.2 mL of the pooled pellet was resuspended and embedded in 12 mL of sterile seaweed alginate suspension at 1% filtered through 0.8 μm, 0.45 μm and finally 0.2 μm (Protanal LF 10/60 FT purchased from FMC Biopolymer) (adapted from the protocol described by Moser et al., 2009). Embedding bacteria in alginate is a technique developed to retain a sufficient number of bacteria in the lungs over an extended time without immunosuppression (Sonderholm et al., 2017) and in case of R. mucilaginosa it also prevents that clumps larger than 0.25 mm are present and consequently limits the risk of blocking the airways of the mice. The suspension was placed in a 20 mL syringe and forced through a nozzle with a coaxial jet of air blowing to create alginate droplets (Nisco encapsulating unit VarJ30). The alginate droplets were collected in a solution of 0.1 M CaCL Tris-HCI buffer (0.1 M, pH 7.0). After 1 hour of stirring, the resulting <30 μm alginate beads were washed twice in 0.9% NaCl with 0.1 M CaCl₂. The number of bacteria embedded in the alginate beads was determined using plate counts on TSA. Before intratracheal challenge, mice were anesthetized with isoflurane (Halocarbon, Norcross, GA) and all efforts were made to minimize suffering. Anesthetized mice were inoculated with R. mucilaginosa-containing alginate beads in 50 μL PBS. The negative control consisted of empty beads produced in the same way as described but without bacteria inside, and the positive control (LPS) was added together with empty alginate beads. Mice were instilled with 10 μg/50 μL LPS with or without R. mucilaginosa embedded in agar beads for 48 hours. Mice were observed during this 48 hours for fur quality, posture, state of activity, and respiratory symptoms and were weighted every 24 hours. Mice were euthanized by cervical dislocation 48 hours post-infection. The left lung was homogenized and used for determination of the microbial load (by plate counting) as well as for cytokine quantification. The spleen and medial lobe of the liver were collected and homogenized to check for dissemination of the instilled bacteria (by plate counting). The right lung was collected in 1 mL of 4% PFA for histologic analysis. Sections for histological analysis were stained by H&E and were examined blindly and scored as previously described (Cigana et al., 2016), using an EVOS FL Auto microscope (Life Technologies).

Quantification of Rothia sp. in respiratory samples. Archived, induced sputum samples were obtained from 85 adults with confirmed bronchiectasis and with a history of two or more infective exacerbations in the preceding year, recruited as part of the BLESS randomized controlled trial (Serisier et al., 2013). Induced sputum was collected at baseline, prior to any intervention and all patients had a sputum neutrophil abundance of ≥70% (as a percentage of total sputum cells). All patients had a chronic lower airway infection. The relative abundance of Rothia sp. was measured in 79/85 samples using 16S rRNA gene amplicon sequencing, as described previously (Rogers et al., 2014). R. mucilaginosa absolute abundance was measured by quantitative PCR (qPCR) in 82/85 samples using the Qiagen Microbial DNA qPCR Assay for R. mucilaginosa (Catalog No.—BPID00297A). Gene copy numbers were determined by comparing to a standard curve as previously described (Taylor et al., 2019).

Inflammatory marker quantification. For in vitro experiments, IL-8 secretion was measured in the cell culture supernatant by a Human IL-8 ELISA MAX™ Standard assay (Biolegend, San Diego, CA) according to the manufacturer's instructions. IL-6, tumor necrosis factor (TNF)-α, IL-1β, interferon (IFN)-γ, granulocyte-macrophage colony stimulating factor (GM-CSF), IL-10, IL-17A and monocyte chemoattractant protein (MCP)-1 levels in cell culture supernatant were quantified by Bioplex Multiplex assays (Bio-Rad, Hercules, CA) according to the manufacturer's instructions. For in vivo animal experiments, Macrophage Inflammatory Protein (MIP)-2 secretion was measured in lung homogenate by a MIP-2 Mouse ELISA kit (LabNed, Amstelveen, NL). Similar to in vitro experiments, IL-1α, IL-4, IL-5, IL-6, IL-10, IL-17, GM-C SF, MCP-1 and TNF-α concentrations in lung homogenate were measured by Bioplex Multiplex assays (Bio-Rad). Inflammatory markers were measured in the sputum of bronchiectasis patients using ELISA (BD Biosciences, San Jose, CA) for IL-8 and IL-1β, and using Magnetic Luminex Performance Assay multiplex kits (R&D Systems, Minneapolis, MN) for matrix metalloproteinase (MMP)-1, MMP-8, and MMP-9, as described previously (Taylor et al., 2015).

Quantitative reverse-transcription polymerase chain reaction (qRT-PCR) array. 3-D A549 cells were infected with P. aeruginosa PAO1, R. mucilaginosa DSM 20746 or a combination of both bacteria as described above. After 4 hours of infection, 3-D A549 cells were washed three times with HB SS and RNA protect reagent (Qiagen, Hilden, Germany) was added according to the manufacturer's instructions. Total cellular RNA was extracted using the Aurum Total RNA Mini Kit (Bio-Rad) and reverse transcribed into cDNA using the iScript advanced cDNA synthesis kit (Bio-Rad). The expression of various genes involved in inflammation (Table SI) was quantified using the Bacterial infections in normal airways PrimePCR panel (Bio-Rad) and SsoAdvanced SYBR Green supermix (Bio-Rad). qRT-PCR experiments were performed on a Bio-Rad CFX96 Real-Time System C1000 Thermal Cycler. TBP, GAPDH and HPRT1 were used as reference genes to allow normalization of the data. Genes with an insufficient expression level were excluded from further analysis.

Analysis of NF-κB activation. A 3-D epithelial cell model of NT-κB-luciferase-transfected A549 cells (BPS Bioscience, San Diego, CA) was developed. Culture conditions were identical to those described above for the 3-D A549 model. Cells were infected for 4 hours with single cultures of P. aeruginosa or R. mucilaginosa, or with a co-culture of P. aeruginosa and R. mucilaginosa (as described above). Likewise, a screening of NF-κB activation by LPS (100 μg/mL) with or without 36 isolates representing five different Rothia species was performed. After the incubation period, 3-D epithelial cells containing the NF-xB-luciferase reporter were transferred to a black 96-well plate for analysis of luminescence. The One-Step Luciferase Assay System (BPS Bioscience, San Diego, CA) was used to lyse the 3-D epithelial cells and to add the luciferase substrate, D-luciferin, according to the manufacturer's instructions. Luminescence was measured by an EnVision (Perkin Elmer) luminometer.

Western blot analysis. Epithelial cells were collected by centrifugation at 4° C. (6 minutes, 1,200 rpm), washed twice in PBS and resuspended in E1A lysis buffer (50 mM Hepes pH 7.6, 250 mM NaCl, 5 mM EDTA, 0.5% NP-40, supplemented with protease and phosphatase inhibitors (Sigma-Aldrich)). Cells were put on ice for 10 minutes and were then centrifuged at 13,000 rpm for 10 minutes. Supernatants were collected and protein concentrations were determined by a Bradford assay. Equal amounts of protein extract (20 μg) were boiled in Laemmli buffer for 10 minutes, fractioned on 10% SDS-PAGE and then transferred onto nitrocellulose membranes. Specific mouse monoclonal antibodies were used to detect A20 (Santa Cruz Biotechnology, Inc.), NF-κB p65 (Santa Cruz Biotechnology, Inc.) and p-IκB-α (Cell signaling) and a specific goat polyclonal antibody was used to detect I-κB-α (Santa Cruz Biotechnology, Inc.). Antibodies were used in concentrations recommended by manufacturer. After three 10 minute-washes in PBS containing 0.1% TWEEN® 20, the nitrocellulose membrane was incubated with anti-mouse IgG HRP-linked Ab (Santa Cruz Biotechnology, Inc.) or anti-goat IgG HRP-linked Ab (Santa Cruz Biotechnology, Inc.).

Statistical analysis. All experiments were carried out at least in biological triplicate. In vitro infection experiments contained two technical replicates for assessment of the viability, quantification of CFU by plating and quantification of IL-8 and MIP-2 by ELISA. Statistical analysis was performed using SPSS 24.0. The Shapiro-Wilk test was applied to evaluate normality of the data. For normally distributed data, differences between the means were assessed by an independent samples t-test or ANOVA followed by a Dunnett's post hoc analysis. Not normally distributed data were further analyzed by a Kruskal-Wallis non-parametric test or Mann-Whitney test. Statistical significance is assumed when p-values are <0.05. For the correlation analysis between Rothia species abundance (relative or absolute) and cytokine/matrix metalloproteinase concentrations, Pearson's correlation (r) was performed on normally distributed (log transformed) data. Otherwise, Spearman's correlation analysis was performed (rs).

### RESULTS

### R. Mucilaginosa Inhibits the Production of Pro-Inflammatory Cytokines by Lung Epithelial Cells in Vitro

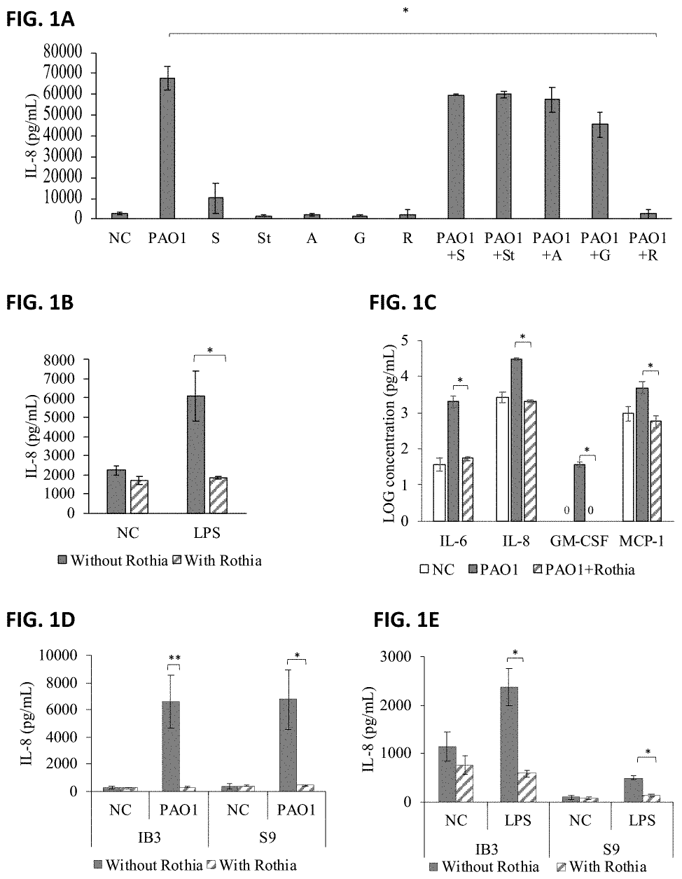

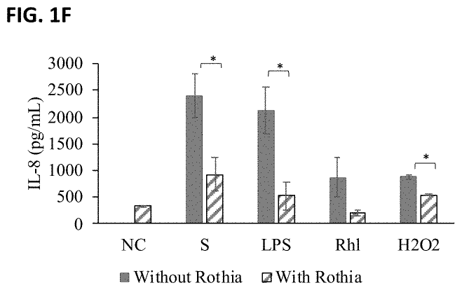

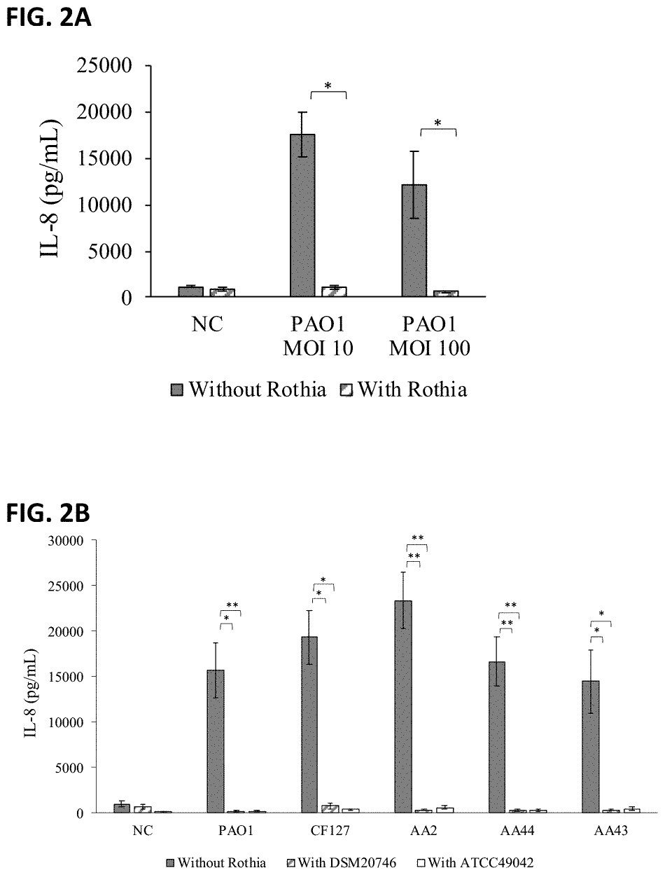

Exposure of an in vivo-like 3-D alveolar epithelial model (A549 cell line) to P. aeruginosa PAO1 (MOI 30:1) induced high IL-8 production, while low or moderate IL-8 production was observed following exposure to S. aureus SP123, S. anginosus LMG14696, A. xylosoxidans LMG26680, G. haemolysans LMG18984 and R. mucilaginosa DSM20746 (FIG. 1A). When 3-D A549 cells were co-exposed to P. aeruginosa PAO1 and one of each of the five other bacterial species, it was observed that R. mucilaginosa completely abolished the P. aeruginosa-induced IL-8 response (FIG. 1A). In contrast, the other members of the lung microbiota did not significantly alter P. aeruginosa-induced IL-8 production. It was observed that R. mucilaginosa exposure also lowered the IL-8 response induced by P. aeruginosa at an MOI of 100:1 and 10:1 (FIG. 2A). For subsequent experiments, an MOI of 10:1 (for each bacterial species) was used. The effect of R. mucilaginosa DSM20746 on P. aeruginosa-induced IL-8 production was confirmed with another strain of R. mucilaginosa (ATCC49042). The R. mucilaginosa strains also reduced the IL-8 response of 3-D A549 cells induced by various P. aeruginosa CF sputum isolates (i.e., CF127, AA2, AA44, AA43) (FIG. 2B). Long-term (24-hour) reduction in IL-8 response by R. mucilaginosa was confirmed using LPS (100 μg/mL) as the pro-inflammatory stimulus (FIG. 1B). These results show that R. mucilaginosa not only reduces IL-8 induced by P. aeruginosa but also that induced by the pure TLR4 ligand, LPS. In addition, R. mucilaginosa reduced cell culture supernatant levels of other pro-inflammatory cytokines (IL-6, IL-8, GM-C SF and MCP-1) induced by P. aeruginosa exposure (FIG. 1C). Evaluation of epithelial cell viability and 3-D model integrity following exposure to P. aeruginosa, R. mucilaginosa or the combination showed normal morphology based on microscopic analysis, and cells retained more than 80% viability after infection.

The observed anti-inflammatory effect of R. mucilaginosa was also confirmed in a 3-D model of CF bronchial epithelial cells (IB-3 cell line) and in the CFTR corrected control (S9 cell line), using P. aeruginosa PAO1 as a pro-inflammatory stimulus for 4 hours (FIG. 1D) or LPS (100 μg/mL) for 24 hours (FIG. 1E). R. mucilaginosa also significantly reduced IL-8 levels promoted by other pro-inflammatory stimuli: S. aureus (MOI 10:1), LPS (100 μg/mL, 4 hours and 24 hours exposure time) and oxidative stress (100 mM H₂O₂) in 3-D A549 cells (FIG. 1F).

### R. Mucilaginosa Lowers the LPS-Induced Pro-Inflammatory Response in an in Vivo Mouse Model

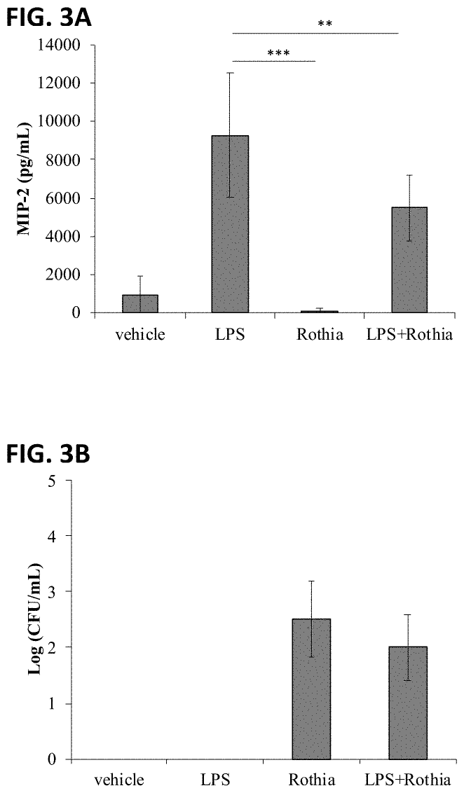

Next, the in vivo anti-inflammatory effect of R. mucilaginosa was evaluated by measuring mouse lung inflammatory cytokine levels following LPS instillation, LPS and R. mucilaginosa (DSM20746) co-exposure, or just R. mucilaginosa exposure. A previously established mouse model of lung infection was used, with bacteria embedded in alginate beads to enable chronic colonization (Cigana et al., 2016). After 48 hours of co-exposure to LPS and R. mucilaginosa, LPS-induced MIP-2 (homologue for human IL-8) was reduced by 40% (FIG. 3A). The lungs of R. mucilaginosa-infected mice contained a similar number of CFU/mL bacteria in the presence and absence of LPS (FIG. 3B). No bacteria were detected in the liver and spleen suggesting no dissemination of R. mucilaginosa to these organs, and mice showed an overall normal fur quality, posture, body weight and no respiratory symptoms in all test conditions.

### Rothia Species Inhibit NF-κB Activation in Epithelial Cells

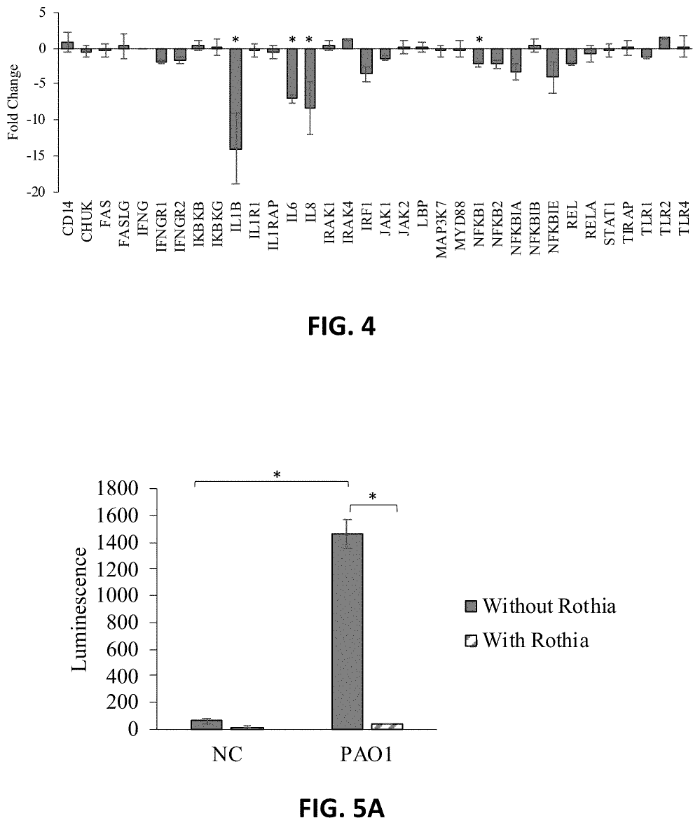

To decipher the mode of action of R. mucilaginosa, the expression of genes that are part of major pro-inflammatory pathways was evaluated in 3-D A549 cells exposed to P. aeruginosa, or P. aeruginosa and R. mucilaginosa. R. mucilaginosa co-exposure significantly downregulated the expression of genes encoding IL-8, IL-6 and pro-IL-1β (FIG. 4) caused by P. aeruginosa, confirming cytokine protein levels in FIG. 1C. In addition, co-exposure to P. aeruginosa and R. mucilaginosa led to a significant downregulation of NFKB1 expression compared to exposure to P. aeruginosa alone (FIG. 4). In addition, regulation of NFKBIA, NFKBIE and REL genes showed a downward trend that further indicates an effect of R. mucilaginosa on the NF-κB pathway.

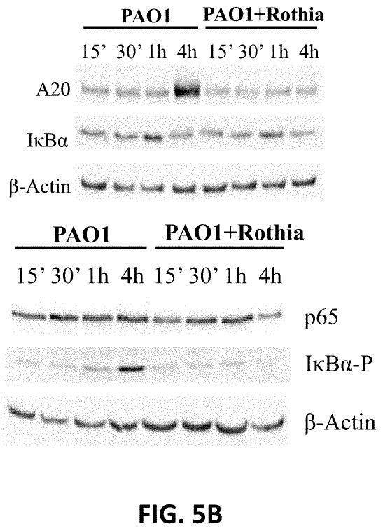

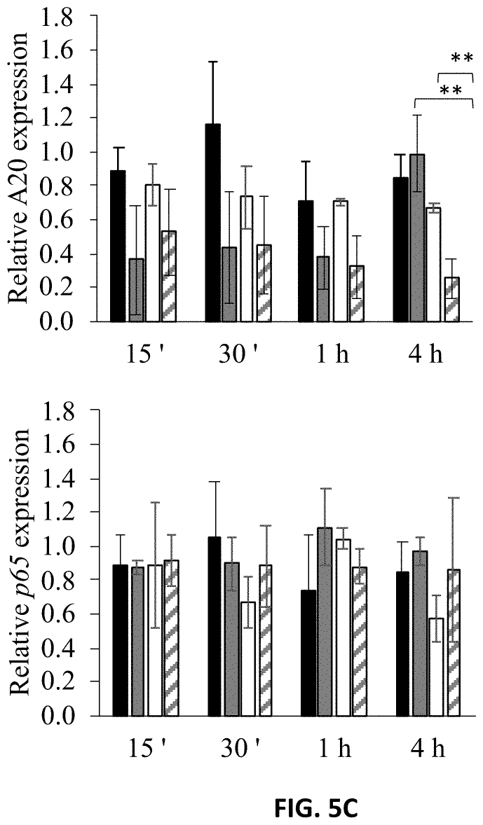

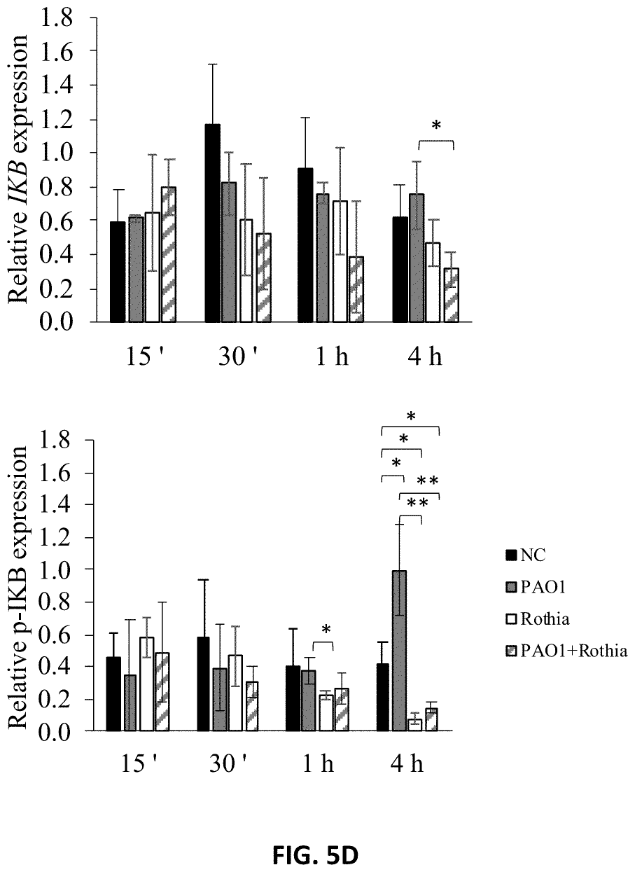

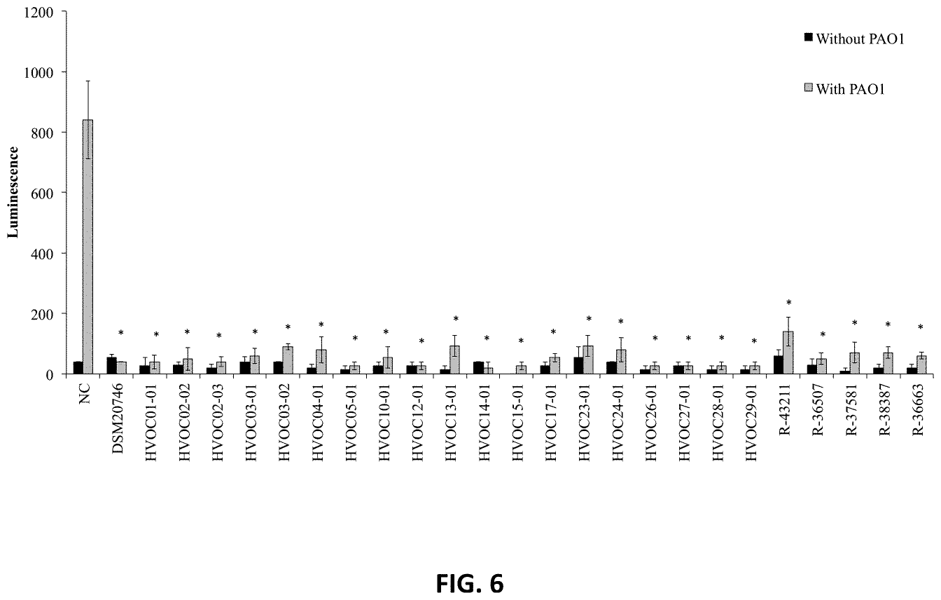

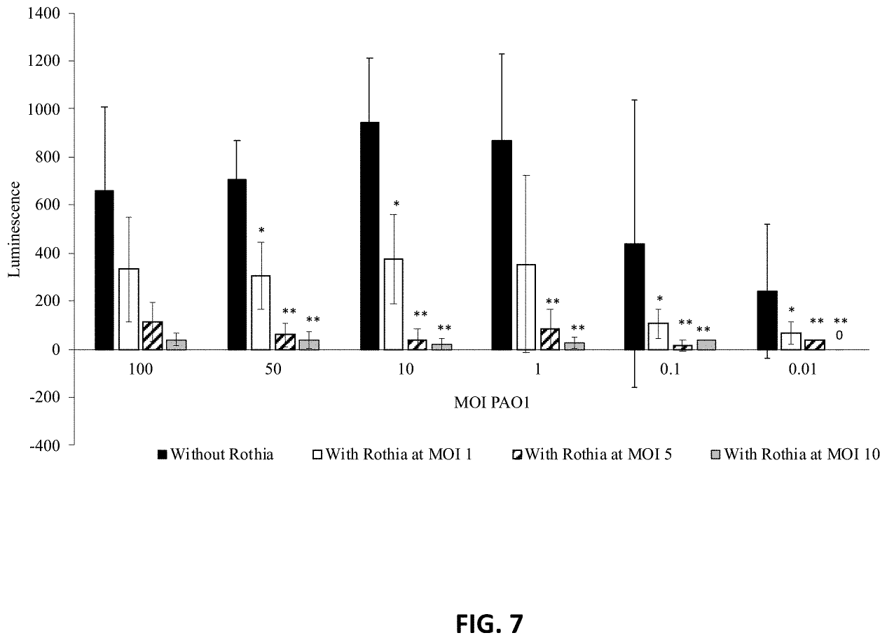

To further explore the influence of R. mucilaginosa on activation of the NF-κB pathway, an NT-κB-luciferase reporter 3-D A549 cell model was used. Stimulation with P. aeruginosa increased the NF-κB activity significantly (FIG. 5A), which was significantly reduced by co-culturing R. mucilaginosa with P. aeruginosa in 3-D A549 cells. The inhibition of the NF-κB pathway by R. mucilaginosa was confirmed using LPS as a pro-inflammatory stimulus and was also observed for 26 isolates representing four different Rothia species from various sources (FIG. 6). In addition, the minimal effective dosage of R. mucilaginosa was evaluated, where an MOI of at least 5:1 of R. mucilaginosa was found to be needed to inhibit P. aeruginosa-induced inflammation (FIG. 7).

Subsequently, a Western blot analysis was performed to investigate at which stage R. mucilaginosa affects NF-κB signaling. 3-D A549 cells were infected with P. aeruginosa or with a co-culture of P. aeruginosa and R. mucilaginosa at various time points (FIGS. 5B and 5C). The presence of R. mucilaginosa prevented P. aeruginosa-induced IκBα phosphorylation, which suggests that R. mucilaginosa prevents NF-κB signaling upstream or at the level of the IκBα kinase complex (IKK). In line with this, the expression of both IκBα and A20, two NF-κB target genes, was greatly reduced in the co-culture samples (FIG. 5C). This further confirms the inhibitory effect of R. mucilaginosa on NF-κB-induced gene expression.

Finally, degradation of IL-8 or inhibition of P. aeruginosa adhesion to 3-D A549 epithelial cells by R. mucilaginosa was excluded as alternative explanations for the observed anti-inflammatory effect (results not shown).

Inverse Relationship Between Airway Inflammation and Rothia Spp. in Patients with Neutrophilic Airways Disease

Two culture-independent approaches were applied to measure Rothia spp. in induced sputum samples from patients with bronchiectasis, a neutrophilic airways disease. The first approach (based on measuring R. mucilaginosa absolute load) indicated that R. mucilaginosa load significantly inversely correlated with levels of IL-8 (r=−0.30), and IL-1β (rₛ=−0.28, as well as with those of metalloproteinases MMP-1 (r=−0.24) and MMP-8 (rₛ=−0.29). There were no significant correlations between R. mucilaginosa and MMP-9 or neutrophil % in sputum. In the second approach, the relative abundance of Rothia was determined and found it also inversely correlated with levels of IL-8 (rₛ=−0.31), IL-10 (rₛ=−0.45), MMP-1 (rs=-0.27), and MMP-8 (rₛ=−0.35). The correlation between Rothia relative abundance and MMP-9 was also significant (rₛ=−0.24). There was again no correlation between Rothia abundance and neutrophil %.

### Example 2: Anti-Inflammatory Activity of Bacteria of the Genera Rothia, Roseomonas and Gemella, and of Consortia of Bacteria

### Materials and Methods

### Bacterial Species and Culturing Conditions

Bacterial isolates representing ten species that are part of the lower airway microbiota of healthy individuals and patients with chronic airway disease, including both bacteria with low and high abundance (Pragman et al., 2012; Romano-Bertrand et al., 2016; Tunney et al., 2008; Harris et al., 2007), were selected and screened for their potential inhibitory effect on LPS-stimulated NF-κB pathway activation (Table 2). Several bacteria were isolated from the oral cavity in the course of the present study. To this end, a swab of the buccal mucosa was taken and was spread on a nutrient agar plate (Lab M, Lancashire, UK) supplemented with 5 mg/mL mupirocin and 10 mg/mL colistin sulphate, and incubated at 37° C. The Ethics Committee of Ghent University authorized and approved the collection of all oral cavity specimens used in this study (file 2019/1554). These isolates were then identified at the species level using matrix assisted laser desorption/ionization time-of-flight mass spectrometry (MALDI-TOF MS) with the commercially available Bruker MALDI Biotyper system (Biotyper reference database version 4.1.80, Bruker Daltonics Inc., Billerica, MA). Liquid cultures of all bacterial species were grown at 37° C. under constant shaking conditions (250 rpm) in Brain Heart Infusion (BHI) broth (Lab M, Lancashire, UK) until stationary phase. For liquid cultures of anaerobic species, BHI was supplemented with 5 mg/L hemin, 10 mg/L NAD, and 0.002 mM vitamin K1 and incubated in anaerobic conditions (AnaeroGen Compact System, Thermo Fisher Scientific).

G. haemolysans cultures were supplemented with 40 mM mannose and were incubated in microaerophilic conditions (5% O₂, 15% CO₂; CampyGen Compact system, Thermo Fisher Scientific).

TABLE 2
Bacterial cultures

| Growth | | | |
|---|---|---|---|
| conditions | Strain | Isolation Site | Source |
| Anaerobic | Fusobacterium nucleatum DSM19507 | Periodontal pocket | DSMZ |
| | Prevotella melaninogenica DSM7089 | Sputum | DSMZ |
| | Veillonella parvula DSM2007 | Mouth | DSMZ |
| Microaerophilic | Gemella haemolysans LMG18984 | Sputum | BCCM/LMG |
| | Gemella haemolysans P9_ANAE15 | Sputum | Own isolate |
| Aerobic | Actinomyces oris HVOC18-01 | Oral cavity | Own isolate |
| | | (buccal mucosa) | |
| | R. dentocariosa HVOC18-02 (LMG | Oral cavity | BCCM/LMG |
| | 31869) | (buccal mucosa) | |
| | Corynebacterium pseudodiphtheriticum | Oral cavity | Own isolate |
| | HVOC02-04 | (buccal mucosa) | |
| | Corynebacterium durum HVOC15-02 | Oral cavity | Own isolate |
| | | (buccal mucosa) | |
| | Roseomonas gilardii LMG24552 | Blood | BCCM/LMG |
| | Rothia mucilaginosa DSM20746 | Throat | DSMZ |
| | Rothia mucilaginosa P1AES | Sputum | Own isolate |
| | Rothia mucilaginosa P5MA1 | Sputum | Own isolate |
| | Rothia terrae HVOC 29-01 | Sputum | Own isolate |
| | Pseudomonas aeruginosa PAO1 | Wound | ATCC |
| | (ATCC 15692) | | |
| | Staphylococcus aureus SP123 | Sputum of | Vandecandelaere |
| | | mechanically- | et al. 2012 |
| | | ventilated patient | |

DSMZ: German Collection of Microorganisms and Cell Cultures, Leibniz Institute, Germany; BCCM/LMG: BCCM/LMG Bacteria Collection, Ghent University, Ghent, Belgium;
ATCC: American Type Culture Collection, Manassas, VA, US

### 3-D Lung Epithelial Cell Culture Models

A previously developed organotypic three-dimensional (3-D) cell culture model of a lung epithelial cell line was used to study the immune response to LPS stimulation (Crabbé et al., 2016; Barrila et al., 2010; Harris et al. 2007; Chen et al., 2019). 3-D cells of the human adenocarcinoma alveolar lung epithelial cell line A549 transfected with an NF-κB-luciferase reporter (BPS Bioscience, San Diego, CA) were prepared as described previously for A549 cells (Crabbé et al., 2016; Barrila et al., 2010; Harris et al. 2007; Chen et a., 2019). Briefly, a monolayer of NF-κB-A549 cells was grown in T75 cell culture flasks containing GTSF-2 medium (HyClone, Logan, UT) supplemented with 1.5 g/L sodium bicarbonate (Sigma-Aldrich), 10% fetal bovine serum (FBS, Life technologies), 2.5 mg/L insulin transferring sodium selenite (Lonza) and 1% penicillin-streptomycin (pen-strep, Life Technologies) at 37° C. under 5% CO₂. At confluence, the cells were seeded in the Rotating Wall Vessel (RWV) bioreactor with type-1 collagen coated dextran beads at a cell to bead ratio of 2:1 and were cultured for 11 to 14 days. The 3-D aggregates were transferred to 48-well plates at a concentration of 2.5×10⁵ cells/well containing fresh serum-free GTSF-2 medium on the day of the infection studies. Prior to infection, bacterial cultures were centrifuged and resuspended in host cell culture medium, i.e., serum-free GTSF-2 (HyClone, Logan, UT) supplemented with 1.5 g/L sodium bicarbonate (Sigma-Aldrich) and 2.5 mg/L insulin transferring sodium selenite (Lonza).

### Analysis of NF-κB Activation

The NF-κB pathway was activated in 3-D NF-κB -A549 cells by 4 hours stimulation with LPS (100 μg/mL) in the presence or absence (positive control) of each of the isolates (Table 1) at varying multiplicity of infection (MOI) starting from an MOI of 100 to an MOI of 1.56 by performing 1:2 serial dilutions. When indicated, cells were exposed to the bacterial isolates in the presence of acetate (at 28.4 mM or 18 mM). After 4 hours incubation, 3-D epithelial cells containing the NF-κB -luciferase reporter were transferred to a black 96-well plate for analysis of luminescence. The One-Step Luciferase Assay System (BPS Bioscience, San Diego, CA) was used to lyse the 3-D epithelial cells and to add the luciferase substrate, D-luciferin, according to the manufacturer's instructions. Luminescence was measured by an EnVision (Perkin Elmer) luminometer.

### Cytotoxicity Assays

3-D cell viability was assessed using the lactate dehydrogenase (LDH) assay. After infection, cell culture supernatant was centrifuged (6 minutes, 1,200 rpm) and 5 μL of supernatant was used for detection of LDH released from epithelial cells in co-culture with LPS and/or bacteria. LDH levels were measured using the LDH detection kit (Sigma-Aldrich) according to the manufacturer's instructions.

### Bacterial Cell Adherence Analysis

To evaluate the number of bacterial cells that associated with 3-D lung epithelial cells with or without LPS, bacterial adherence to the host cells was determined as described previously (Anderson et al., 2008; Crabbé et al., 2017; Tran et al., 2014). In short, 3-D epithelial cell aggregates were transferred to a new 48-well plate and washed twice with HBSS. Bacterial cells were dissociated from epithelial cells using 0.1% TRITON® X-100 and then quantified by plating on Columbia agar supplemented with 6% sheep blood for anaerobic and microaerophilic strains and on nutrient agar for aerobic strains.

### Short-Chain Fatty Acid (SCFA) Quantification

Short-chain fatty acids (SCFA) analysis was performed according to Andersen et al. (2014). C2-C8 fatty acids, including isoforms C4-C6, were measured by gas chromatography (GC-2014, Shimadzu®, The Netherlands) with a DB-FFAP 123-3232 column (30 m×0.32 mm×0.25 μm; Agilent, Belgium) and a flame ionization detector (FID). Liquid samples were conditioned with sulfuric acid and sodium chloride and 2-methyl hexanoic acid as internal standard for quantification of further extraction with diethyl ether. The prepared sample (1 μL) was injected at 280° C. with a split ratio of 60 and a purge flow of 3 mL/minute. The oven temperature increased by 6° C./minute from 110° C. to 158° C. and by 8° C./minute from 158° C. to 175° C. where it was kept for 1 minute. FID had a temperature of 220° C. The carrier gas was nitrogen at a flow rate of 2.49 mL/minute.

### Determining Synergism Between Anti-Inflammatory Bacteria

To determine a potential synergistic effect, a checkerboard assay (Stein et al., 2015) was performed using selected aerobic bacteria (i.e., R. mucilaginosa, R. dentocariosa, and R. gilardii) mixed together at varying MOI (range 20-0.1) by applying 1:2 serial dilutions. Next, NE-κB activation was analyzed after 4 hours aerobic stimulation with LPS (100 μg/mL) in the presence or absence of each of the mixes was determined as described above. Using SPSS 24.0, p-values were calculated by a one-way ANOVA followed by a Dunnett's post hoc analysis, hereby comparing the luminescence obtained in the presence of each mix to the luminescence generated by the positive control (i.e., 100 μg/mL). The triple combination of aerobic bacteria (mix 1) containing the lowest number of bacteria per species (minimal effective MOI, mMOI) that exerts a significant downregulation of NF-κB activation in LPS-stimulated 3-D epithelial cells, was determined. The fractional inhibitory concentration index (FICI) for this triple combination (mix 1) was calculated based on the protocol described by Stein et al. 2015:

$$F I C I = \frac{m M O {I}_{A} ( mix )}{m M O {I}_{A} ( single )} + \frac{m M O {I}_{B} ( mix )}{m M O {I}_{B} ( single )} + \frac{m M O {I}_{C} ( mix )}{m M O {I}_{C} ( single )}$$

The interpretation of FICI was done as proposed by Odds 2003: FICI <0.5, synergy; 0.5<FICI<4, no interaction; and FICI≥4 antagonism. Next, G. haemolysans was added to the selected triple bacterial combination at various MOIs (range 100-1.56), and the mMOI of G. haemolysans in this mix was determined. The FICI value of this second mix (mix 2) containing four bacterial species (i.e., R. mucilaginosa, R. dentocariosa, R. gilardii, and G. haemolysans) was calculated using the same formula as stated above. In addition, consortia of two species were tested for synergistic anti-inflammatory activity, using the same approach as described above. The FICI was calculated using the following formula:

$$F I C I = \frac{m M O {I}_{A} ( mix )}{m M O {I}_{A} ( single )} + \frac{m M O {I}_{B} ( mix )}{m M O {I}_{B} ( single )}$$

### Testing of Growth Stimulating Carbon Sources for Anti-Inflammatory Bacteria

Using Phenotype Microarray (PM) panels (Biolog), the influence of 190 carbon sources on the growth of the potential probiotic species R. mucilaginosa, R. dentocariosa, R. gilardii, G. haemolysans, and common respiratory pathogens P. aeruginosa, and S. aureus was determined. PM1 and PM2 panels (Biolog) were inoculated according to the manufacturer's instructions. Growth curves were generated for 35 hours by measuring the absorbance at 590 nm using an EnVision (Perkin Elmer) plate reader.

### Statistical Analysis

All experiments were carried out at least in biological triplicate. Infection experiments contained two technical replicates for assessment of the viability and quantification of CFU by plating. Statistical analysis was performed using SPSS 24.0. The Shapiro-Wilk test was applied to evaluate normality of the data. For normally distributed data, differences between the means were assessed by an independent samples t-test or ANOVA followed by a Dunnett's post hoc analysis. Not normally distributed data were further analyzed by a Kruskal-Wallis non-parametric test or Mann-Whitney test. Statistical significance is assumed when p-values are <0.05.

### RESULTS

### Commensal Bacteria Exhibit Anti-Inflammatory Properties

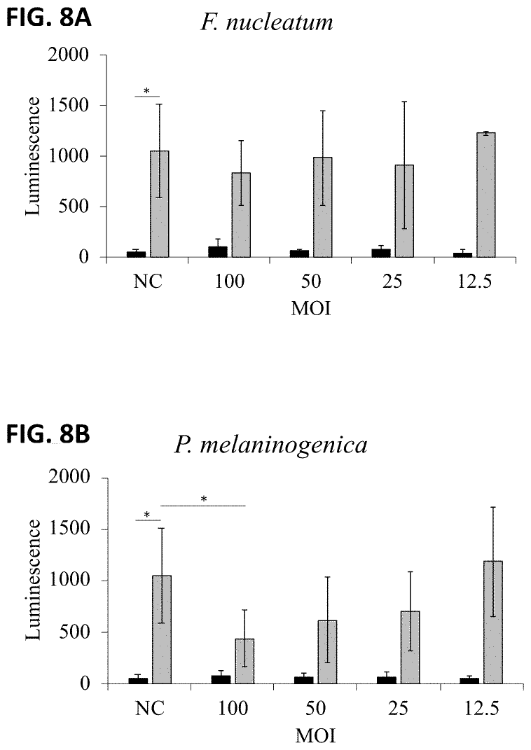

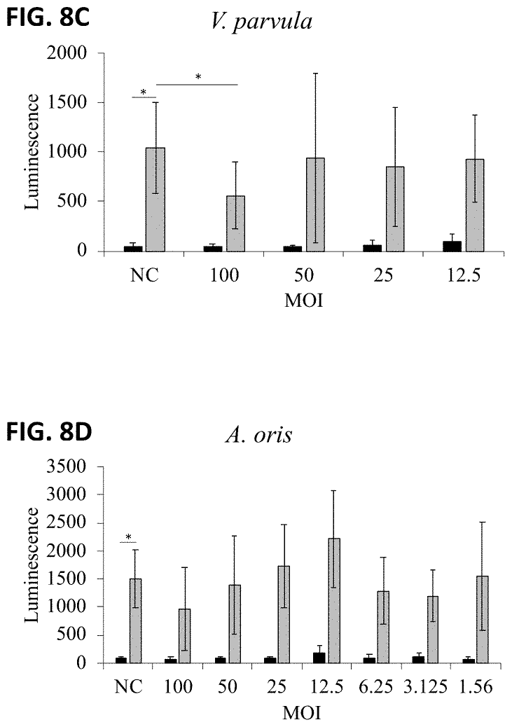

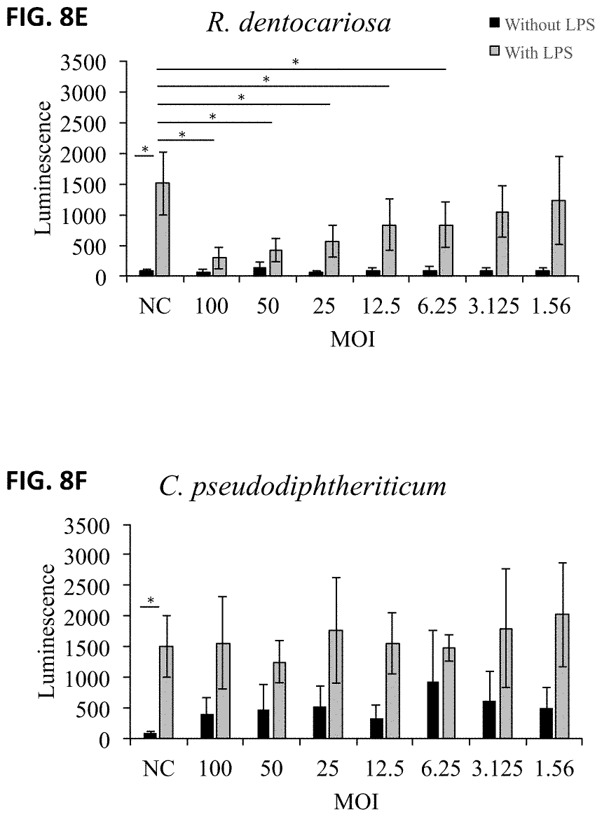

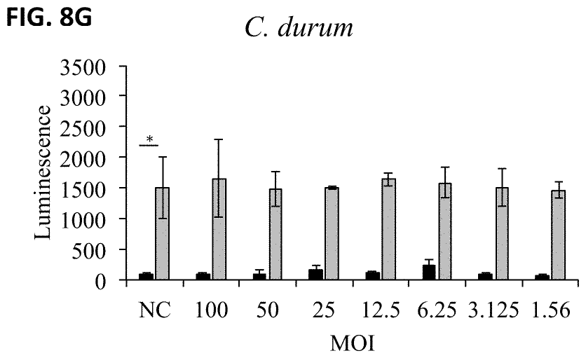

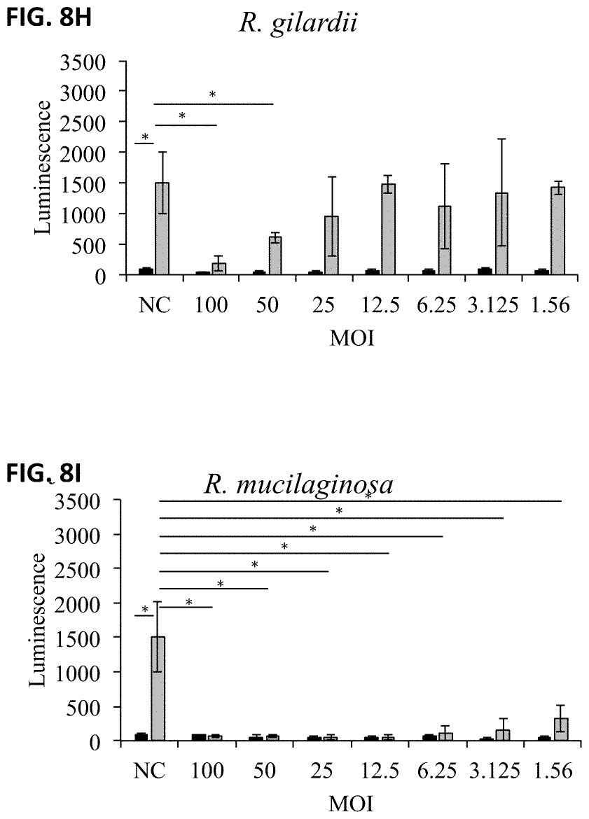

Exposure of 3-D lung epithelial cells to LPS significantly induced NF-κB pathway activation compared to unstimulated 3-D A549 cells, while single cultures of each of the candidate anti-inflammatory bacterial species (Table 3) did not activate the NF-κB pathway at varying MOI (range 100-1.56) (FIGS. 8A-8I). Adhesion of the candidate anti-inflammatory bacteria to 3-D lung epithelial cells with and without LPS was subsequently evaluated and it was observed that bacterial association to host cells was not influenced by LPS. Next, epithelial cell viability was tested following a 4-hour exposure to LPS with or without the candidate anti-inflammatory bacterial isolates, finding less than 10% cell death based on LDH release.

In order to study the potential of candidate anti-inflammatory bacterial species to lower this LPS-induced NF-κB pathway activation, 3-D A549 cells were co-exposed to LPS and each of the bacteria (FIGS. 8A-8I, Table 3). The anaerobic bacteria P. melaninogenica and V. parvula significantly diminished LPS-induced NF-κB pathway activation when administered at an MOI of 100 while lower doses of P. melaninogenica and V. parvula and all tested doses of F. nucleatum did not reduce this activation. The microaerophilic bacterium G. haemolysans significantly reduced LPS-induced NF-κB pathway activation at an MOI>25. When administered at an MOI>50 LPS-induced NF-κB pathway activation was reduced to background levels (i.e., not significantly different from unexposed control). Of the seven aerobic species tested, three did not show any effect on LPS-induced NF-κB pathway activation (A. oris, C. pseudodiphtheriticum, and C. durum). R. mucilaginosa showed the most pronounced effect and significantly reduced LPS-induced NF-κB pathway activation at an MOI as low as 1.56 and was able to completely abolish NF-κB pathway activation at an MOI of 3.125. Administration of R. dentocariosa significantly reduced LPS-induced NF-κB pathway activation at MOI≥6.25, while for R. gilardii an MOI≥50 was required for the same effect. Furthermore, R. gilardii was able to completely abolish the NF-κB pathway activation at an MOI of 100.

TABLE 3
Influence of commensal bacteria on NF-κB-mediated inflammation
in 3-D A549 lung epithelial cells. Summary of anti-inflammatory
potential of each bacterium at each tested MOI.

| | MOI | | | | | | |
|---|---|---|---|---|---|---|---|
| | 100 | 50 | 25 | 12.5 | 6.25 | 3.125 | 1.56 |
| F. nucleatum DSM 19507 | − | − | − | − | n.t. | n.t. | n.t. |
| P. melaninogenica DSM 7089 | + | − | − | − | n.t. | n.t. | n.t. |
| V. parvula DSM 2007 | + | − | − | − | n.t | n.t. | n.t. |
| G. haemolysans LMG 18984 | ++ | ++ | + | − | n.t. | n.t. | n.t. |
| A. oris HVOC 18-01 | − | − | − | − | − | − | − |
| R. dentocariosa HVOC 18-02 | + | + | + | + | + | − | − |
| C. pseudodiphtheriticum HVOC | − | − | − | − | − | − | − |
| 02-04 | | | | | | | |
| C. durum HVOC 15-02 | − | − | − | − | − | − | − |
| R. gilardi LMG 24552 | ++ | + | − | − | − | − | − |
| R. mucilaginosa DSM 20746 | ++ | ++ | ++ | ++ | ++ | ++ | + |
| R. mucilaginosa P1AE5 | n.t. | n.t. | n.t. | n.t. | + | + | + |
| R. mucilaginosa P5MA1 | n.t. | n.t. | n.t. | n.t. | + | + | + |
| R. terrae HVOC 29-01 | n.t. | n.t. | n.t. | n.t. | + | + | + |
| G. haemolysans P9 ANAE15 | n.t. | n.t. | + | + | − | − | − |

(−) no significant reduction of LPS-stimulated NF-κB pathway activation;
(+) significant reduction of LPS-stimulated NF-κB pathway activation (p < 0.05 compared to LPS-stimulated control but p < 0.05 compared to non-exposed control, i.e., still significantly different from background);
(++) significant reduction to background levels of LPS-stimulated NF-κB pathway activation (p < 0.05 compared to LPS-stimulated control and p > 0.05 compared to non-exposed control, i.e., not significantly different from background);
(n.t.) not tested, N ≥ 3.

### Anti-Inflammatory Properties are Not Due to Production of Short Chain Fatty Acids (SCFA)

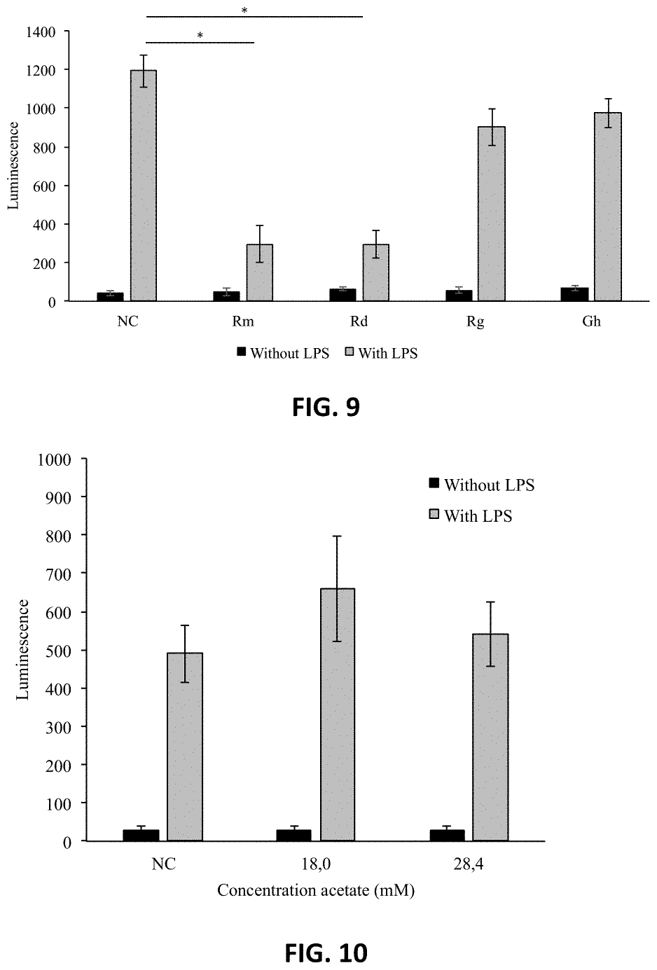

The role of SCFA was investigated, anti-inflammatory compounds secreted by many gut probiotic strains (Li et al., 2018), in the anti-inflammatory effect of species that showed activity in their cell-free supernatant, i.e., R. mucilaginosa and R. dentocariosa (FIG. 9). Subsequently, the levels of SCFAs were quantified in the supernatants of these two species. For all tested SCFAs (i.e., acetate, propionate, isobutyrate, butyrate, isovalerate and valerate), the concentration in the supernatant did not differ significantly between the four strains investigated (Table 4). Next, tests were performed to see if the SCFA with the highest concentration in all samples (i.e., acetate) could exert anti-inflammatory effects in the concentration range detected in the bacterial supernatants. The highest (i.e., 28.3 mM produced by R. mucilaginosa DSM20746) and lowest (i.e., 18.0 mM produced by R. dentocariosa HVOC18-02) concentration of acetate did not reduce LPS-stimulated NF-κB pathway activation in the model system (FIG. 10).

TABLE 4
Quantification of SCFA

| SCFA (mM) | R. mucilaginosa | R. dentocariosa | A. oris |
|---|---|---|---|
| Acetate | 28.36 ± 5.77 | 18.06 ± 2.30 | 18.37 ± 8.67 |
| Proprionate | 0.12 ± 0.103 | 0.20 ± 0.002 | 0.20 ± 0.004 |
| isoButyrate | 0.17 ± 0.006 | 0.17 ± 0.002 | 0.16 ± 0.004 |
| Butyrate | 0.17 ± 0.002 | 0.19 ± 0.003 | 0.17 ± 0.001 |
| isoValerate | 0.14 ± 0.004 | 0.13 ± 0.003 | 0.13 ± 0.001 |
| Valerate | ND | ND | ND |

Data represent SCFA mean concentration (mM) ± StDev, ND = not detected, n = 3.

### Synergistic Activity of Anti-Inflammatory Bacteria

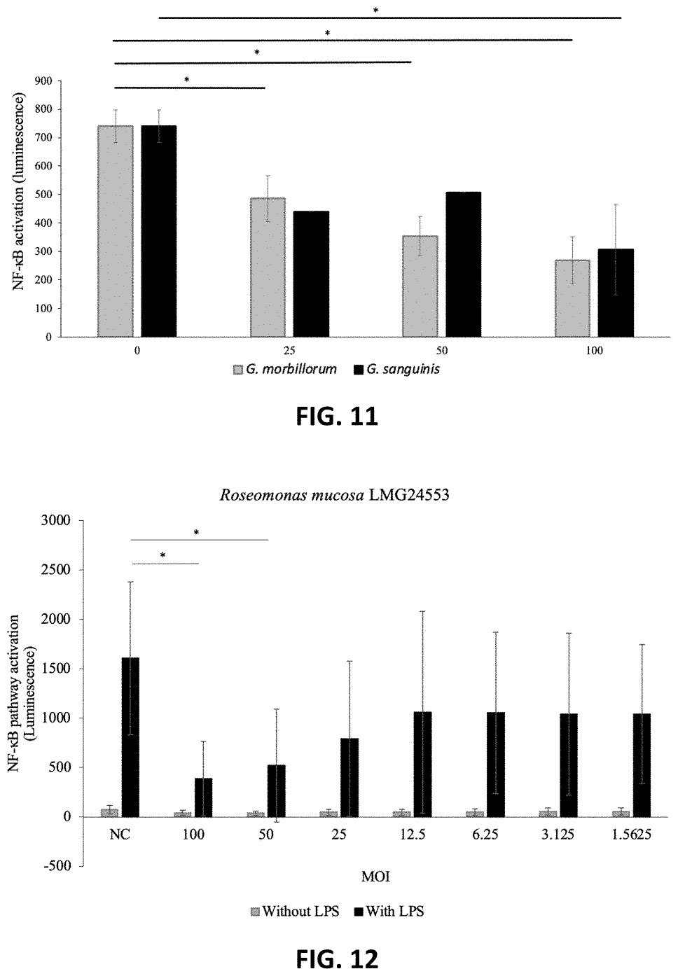

Since the selected anti-inflammatory species are absent in the healthy lungs or present in low numbers (Marsh et al., 2018), it is preferable to explore their therapeutic potential using the lowest dose possible. Even in the lungs of patients with chronic respiratory diseases, such as CF patients, a maximum load corresponding to an of MOI 120 is reached, though in the COPD lungs a much lower MOI (i.e., MOI 0.05) is found (Sze et al., 2012). Therefore, the bacteria with an anti-inflammatory effect at an MOI higher than 100 (i.e., P. melaninogenica and V. parvula) were excluded from further analysis while the more potent anti-inflammatory bacteria (i.e., R. mucilaginosa, R. dentocariosa, R. gilardii, and G. haemolysans) were evaluated further for synergistic anti-inflammatory effects when cultured as a consortium. It was previously shown that different species of the genus Rothia were anti-inflammatory. Two isolates of Rothia mucilaginosa (strain P1AE5, P5MA1) and one strain of Rothia terrae (HVOC 29-01) were included and all tested MOIs exhibited an anti-inflammatory effect (Table 3). To evaluate if the observed anti-inflammatory effect of G. haemolysans and R. gilardii can also be observed for other species belonging to the respective genera, additional species were tested, confirming that G. morbillorum (LMG18985; BCCM/LMG) and G. sanguinis (in house isolate out of sputum) inhibited inflammation induced by LPS at an MOI of 25 and 100, respectively (FIG. 11). The anti-inflammatory effect of another strain of G. haemolysans P9_ANAE15 (in house isolate from sputum) was also evaluated at an MOI of 12.5 was needed for the effect (Table 3). Roseomonas mucosa (LMG24553; BCCM/LMG) as found to be anti-inflammatory at an MOI of 50 (FIG. 12).

Next, a checkerboard assay (Stein et al., 2015) was carried out using R. mucilaginosa, R. dentocariosa, R. gilardii, and G. haemolysans to determine the MOI of each bacterium in the mix that is required to exert a significant reduction in LPS-stimulated NF-κB pathway activation compared to the positive control (LPS only) (minimal effective MOI, mMOI).

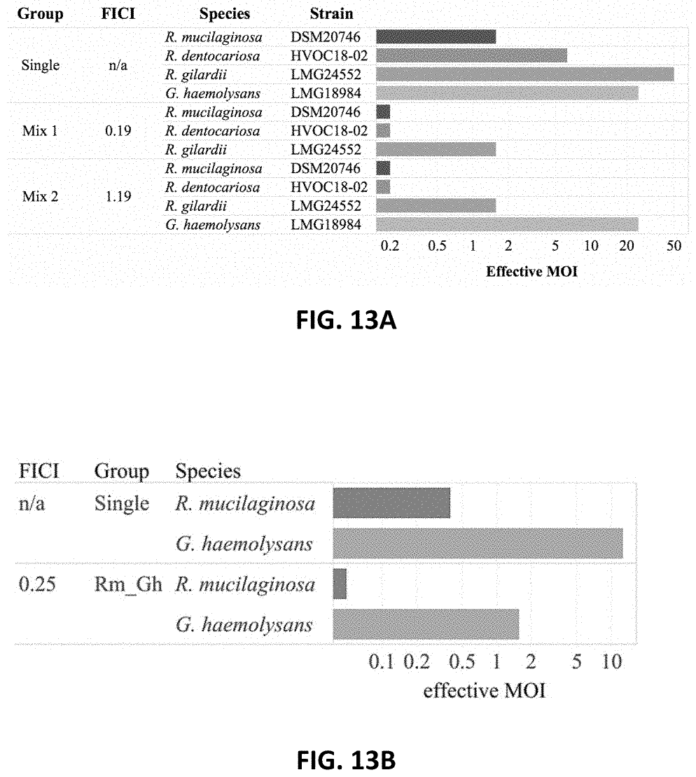

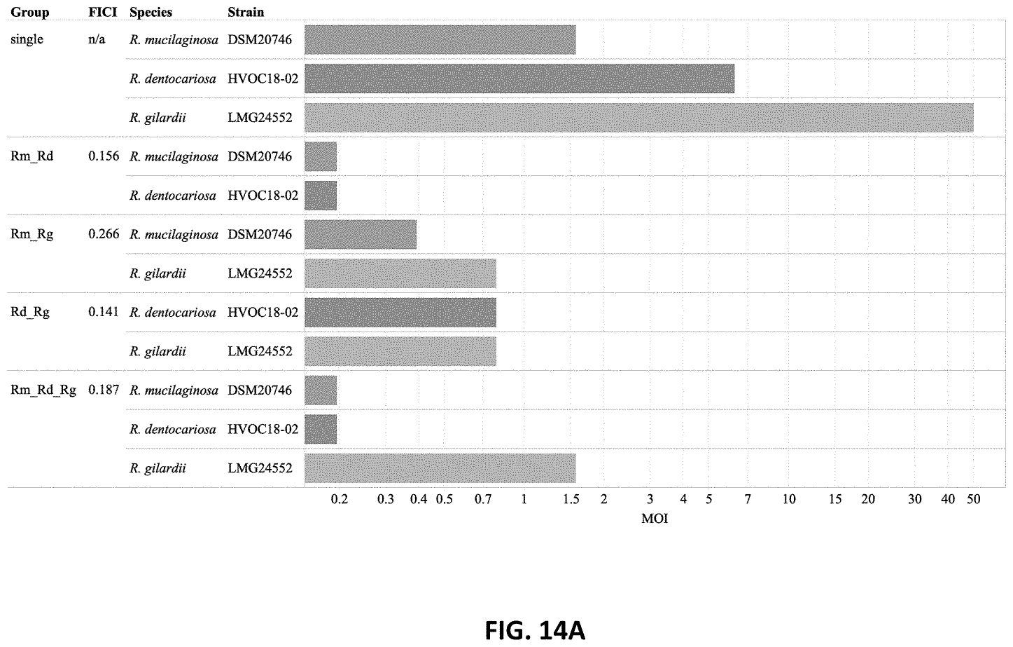

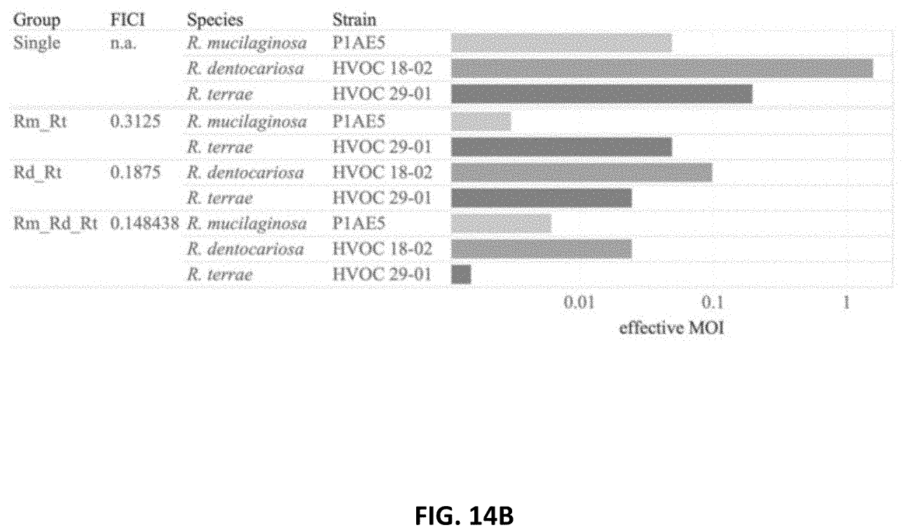

Firstly, R. mucilaginosa, R. dentocariosa and R. gilardii were co-cultured in mixes of varying MOI in a 1:2 serial dilution starting from an MOI of 100 to an MOI of 0.10. In this mix the MOI that caused a significant reduction in LPS-stimulated NF-κB pathway activation was 0.20, 0.20, and 1.56 for R. mucilaginosa, R. dentocariosa and R. gilardii, respectively, indicating that combining bacteria allowed to strongly reduce the MOI for each species needed to exert an anti-inflammatory effect (i.e., 8-fold for R. mucilaginosa, and 32-fold for both R. dentocariosa and R. gilardii) (FIG. 13A). In addition, this mix reduced the LPS-stimulated NF-κB pathway activation to background levels (i.e., not significantly different from the unstimulated negative control; p-value=0.27) while keeping the total number of bacteria in this mix slightly lower than that of the most potent bacterial species (i.e., R. mucilaginosa) required for the same effect (i.e., total MOI of 1.96 for mix 1 vs. MOI of 3.13 for R. mucilaginosa). Calculation of the FICI revealed that the three bacteria in this mix (mix 1) work synergistically (FICI=0.19) (FIG. 13A). Adding varying MOIs of G. haemolysans to mix 1 under micro-aerobic conditions (5% O₂) did not increase synergistic activity (FICI=1.19) (FIG. 13A), however, it was observed that G. haemolysans P9_ANAE15 and R. mucilaginosa P5MA1 exhibited synergistic activity (FICI=0.25) (FIG. 13B). Tests were also performed to see whether consortia of two bacterial species exerted synergistic anti-inflammatory activity, and it was found for the three possible combinations of two species that less bacteria per species were needed to exert the effect (FIGS. 14A and 14B). Accordingly, the FICI values for these combinations indicated synergism. Finally, tests were performed to see if three species of the same genus (R. mucilaginosa, R. dentocariosa, and R. terrae) could also exert synergistic activity and found that this was the case, with a FICI value as low as 0.15. Also, synergistic activity was observed when R. terrae was combined with either R. mucilaginosa or R. dentocariosa. It is believed that this study is the first to demonstrate the synergistic activity of local/lung probiotics in the context of inflammation, in particular, lower respiratory tract inflammation.

### Carbon Sources as Potential Prebiotics to Stimulate Growth of Anti-Inflammatory Bacteria

As there is increasing interest in the exploration of prebiotics to enhance the growth of probiotics and/or increase the production of their beneficial compounds, the possibility of inducing the growth of R. mucilaginosa, R. dentocariosa, R. gilardii, and G. haemolysans without inducing the growth of the common pathogens P. aeruginosa and/or S. aureus was assessed. Using the Biolog Phenotype Microarray PM1 and PM2 assays, 190 carbon sources were screened. After 35 hours of growth, the area under the curve (AUC) was calculated (Table 5). Carbon sources for which the AUC values were lower than 50 were considered as not supporting growth and were omitted from further data analysis. Although the results are preliminary (n=1), one carbon source, namely D,L-glycerol-phosphate, showed a high AUC for at least one anti-inflammatory bacterium, while not or only minimally supporting growth of both S. aureus and P. aeruginosa. Next, carbon sources that could not be used by either P. aeruginosa or S. aureus were selected. While twelve carbon sources did not support growth of P. aeruginosa (i.e., β-methyl-D-glucoside, D-trehalose, glycerol, maltose, maltotriose, N-acetyl-β-D-mannosamine, N-acetyl-D-glucosamine, sucrose, dextrin, oxomalic acid, salicin, and turanose), all carbon sources that supported the growth of anti-inflammatory bacteria also allowed growth of S. aureus. Of these twelve carbon sources, six (i.e., β-methyl-D-glucoside, D-trehalose, glycerol, maltose, maltotriose, and sucrose) allowed growth of both R. mucilaginosa and R. dentocariosa, three (i.e., dextrin, salicin, and turanose) stimulated only the growth of R. dentocariosa, one (oxomalic acid) enabled growth of both R. dentocariosa and R. gilardii, and two (i.e., N-acetyl-β-D-mannosamine and N-acetyl-D-glucosamine) supported growth of G. haemolysans. Hence, it remains challenging to uncover selective prebiotics that do not induce the growth of pathogenic bacteria.

These results indicate the usefulness of the herein identified prebiotics, bacteria (probiotics), and possibly synbiotics (i.e., combination of probiotics and prebiotics) in several inflammatory disease conditions. It could furthermore be considered to combine the use of these pre- pro- or synbiotics with selective antibiotics, such as, e.g., against P aeruginosa and/or S. aureus.

TABLE 5
AUC of growth curves of potential beneficial bacteria and of
pathogens in the presence of different carbon sources (Biolog PM1-2). For cells without values
AUC < 50. n = 1

| | Bacterial species | | | | | |
|---|---|---|---|---|---|---|
| | R. | R. | | G. | P. | |
| Carbon source | mucilaginosa | dentocariosa | R. gilardii | haemolysans | aeruginosa | S. aureus |
| 1,2-Propanediol | | | | | | 150.0 |
| 2-Aminoethanol | | | | | 293.7 | |
| 2-Deoxy-Adenosine | | | | | | 419.1 |
| 2-Deoxy-D-Ribose | | 376.6 | 366.7 | 180.3 | 529.9 | 580.2 |
| 2,3-Butanediol | | | | 145.4 | | 147.2 |
| 3-0-β-D-Galacto-pyranosyl-D- | | | | | 188.8 | 446.3 |
| Arabinose | | | | | | |
| 3-Hydroxy 2-Butanone | | | | | 183.2 | 255.6 |
| 3-Methyl Glucose | | 93.5 | | | | 190.8 |
| 4-Hydroxy Benzoic Acid | | | | | 909.3 | 140.6 |
| 5-Keto-D-Gluconic Acid | | 277.0 | 423.9 | 122.8 | 1312.5 | 432.1 |
| Acetamide | | | | | 123.0 | 93.1 |
| Acetic Acid | 374.3 | 181.2 | 300.8 | | 345.4 | 898.7 |
| Acetoacetic Acid | 301.4 | 229.8 | 172.4 | | 301.3 | 928.3 |
| Adenosine | | | | 65.6 | 398.1 | 472.6 |
| Amygdalin | | | | | | 254.6 |
| Arbutin | | 55.2 | | | | 282.2 |
| Bromo Succinic Acid | | 808.4 | 67.3 | | 102.6 | 430.7 |
| Butyric Acid | | 88.7 | 72.0 | | | 1011.5 |
| Caproic Acid | | | | | | 908.9 |
| Chondroitin Sulfate C | | | | | | 199.7 |
| Citraconic Acid | | | | | | 86.7 |
| Citramalic Acid | | | | | 183.9 | |
| Citric Acid | | | 156.2 | | 846.5 | 169.0 |
| D-Alanine | | | | | 668.1 | 464.9 |
| D-Arabinose | | 305.6 | 356.3 | 88.1 | 763.4 | 705.0 |
| D-Arabitol | | | | | | 832.3 |
| D-Aspartic Acid | | | | | 101.3 | 242.2 |
| D-Cellobiose | | | | | 70.0 | 495.8 |
| D-Fructose | 1111.2 | 109.5 | 315.8 | 88.8 | 413.3 | 992.9 |
| D-Fructose-6-Phosphate | 127.5 | 87.1 | | 134.2 | 783.8 | 437.8 |
| D-Fucose | | 119.9 | 71.4 | | 91.3 | 138.3 |
| D-Galactonic-Acid-γ-Lactone | | | | | 397.9 | |
| D-Galactose | 101.1 | 533.6 | 186.6 | | 720.0 | 834.0 |
| D-Galacturonic Acid | | 57.0 | | | 598.2 | 165.1 |
| D-Gluconic Acid | | | | | 617.8 | 321.0 |
| D-Glucosamine | | 200.6 | 196.5 | 618.4 | 840.0 | 884.6 |
| D-Glucosaminic Acid | | | | | | 267.3 |
| D-Glucose-1-Phosphate | | | | | | 189.0 |
| D-Glucose-6-Phosphate | | | | | 399.6 | 168.0 |
| D-Glucuronic Acid | | | | | 543.2 | 292.7 |
| D-Lactic Acid Methyl Ester | | | | | 1081.7 | 517.2 |
| D-Mannitol | | | | | 420.0 | 884.6 |
| D-Mannose | 1017.6 | 106.1 | | 218.6 | 495.0 | 729.5 |
| D-Melezitose | | 57.2 | | | | 969.2 |
| D-Melibiose | | 249.8 | 86.9 | | | 220.2 |
| D-Psicose | 436.4 | 428.2 | 86.7 | | 771.3 | 698.5 |
| D-Rafinose | | | | | | 282.1 |
| D-Ribono-1,4-Lactone | | | | | | 100.2 |
| D-Ribose | 371.4 | 431.1 | 718.5 | 627.5 | 1450.3 | 769.0 |
| D-Saccharic Acid | | | | | 108.8 | 57.3 |
| D-Serine | | | | | | 76.2 |
| D-Sorbitol | | | | | 129.8 | 412.2 |
| D-Tagatose | | 117.7 | 75.2 | | 574.0 | 633.2 |
| D-Threonine | | | | | | 87.8 |
| D-Trehalose | 572.3 | 937.7 | | | | 852.9 |
| D-Xylose | 157.4 | 257.9 | 310.4 | 581.3 | 1218.3 | 92.4 |
| D,L-Carnitine | | | | | 574.6 | |
| D,L-Malic Acid | | | 169.8 | | 852.7 | 696.0 |
| D,L-α-Glycerol-Phosphate | 247.7 | 271.8 | | | 114.5 | |
| D,L-Octopamine | | | | | 1142.2 | 166.2 |
| Dextrin | | 135.6 | | 66.1 | | 908.1 |
| Dihydroxy Acetone | | 329.6 | 460.5 | 324.4 | 926.8 | 410.9 |
| Dulcitol | | | | | 72.0 | |
| Formic Acid | | | | | | 474.0 |
| Fumaric Acid | | 202.9 | | | 1085.5 | 687.9 |
| Gelatin | | | | | | 52.4 |
| Gentiobiose | | | | | | 471.4 |
| Glucuronamide | 84.9 | 124.2 | 53.3 | 97.6 | 820.8 | 62.8 |
| Glycerol | 786.8 | 998.9 | 59.0 | | 78.3 | 802.4 |
| Glycine | | | | | 101.6 | 743.1 |
| Glycogen | | | | | | 197.7 |
| Glycolic Acid | | | | | | 344.8 |
| Glycyl-L-Aspartic Acid | | | | | 538.0 | 840.3 |
| Glycyl-L-Glutamic Acid | 140.3 | | 61.5 | | 285.6 | 871.5 |
| Glycyl-L-Proline | | 72.5 | | | 387.4 | 292.4 |
| Glyoxylic Acid | 142.0 | 53.5 | 85.2 | | | |
| Hydroxy-L-Proline | | | | | 696.8 | 492.1 |
| Inosine | | | | | 141.0 | 1075.0 |
| Itaconic Acid | | | | | 790.8 | |
| L-Alaninamide | | | | | | 295.4 |
| L-Alanine | | | | | 775.3 | 564.0 |
| L-Alanyl-Glycine | | | 76.2 | 114.6 | 180.2 | 809.1 |
| L-Arabinose | 195.3 | 314.8 | 804.7 | 120.4 | 1269.0 | 356.7 |
| L-Arabitol | | | | | | 165.7 |
| L-Arginine | | | | | 285.4 | 816.4 |
| L-Asparagine | | | | | 1032.0 | 684.0 |
| L-Aspartic Acid | | | | | 730.7 | 464.1 |
| L-Fucose | 155.6 | 478.0 | 86.6 | 95.6 | 701.4 | 277.6 |
| L-Galactonic Acid γ-Lactone | | | 212.2 | | 549.8 | 176.4 |
| L-Glucose | | | | | | 314.7 |
| L-Glutamic Acid | | | | | 788.3 | 644.7 |
| L-Glutamine | | | | | 987.8 | 697.1 |
| L-Histidine | | | | | 773.2 | 467.3 |
| L-Homoserine | | | | | 82.5 | 194.1 |
| L-Isoleucine | | | | | 86.2 | 113.8 |
| L-Lactic Acid | 292.5 | 632.8 | 70.3 | | 349.6 | 773.2 |
| L-Leucine | | | | | 125.5 | 231.5 |
| L-Lysine | | | | | | 331.6 |
| L-Lyxose | 377.5 | 474.8 | 765.9 | 430.7 | 1393.5 | 308.4 |
| L-Malic Acid | | | 202.5 | 73.9 | 808.2 | 648.8 |
| L-Methionine | | | | | 52.6 | 254.7 |
| L-Ornithine | 56.2 | | | | 66.2 | 760.9 |
| L-Phenylalanine | | | | 64.4 | | 167.3 |
| L-Proline | 268.5 | 1049.5 | | | 425.7 | 567.9 |
| L-Pyroglutamic Acid | | | | | 1438.7 | 427.9 |
| L-Rhamnose | 197.8 | 149.9 | 178.6 | 104.6 | 265.3 | 423.5 |
| L-Serine | | | | | | 558.2 |
| L-Sorbose | | 52.1 | | | 94.3 | 458.7 |
| L-Tartaric Acid | | | | | | 163.4 |
| L-Threonine | | | | | | 754.3 |
| Lactitol | | | | | | 338.8 |
| Lactulose | | | | | | 656.2 |
| Laminarin | | | | | | 192.8 |
| m-Hydroxy Phenyl Acetic Acid | | | | | 114.0 | |
| m-inositol | | | | | | 157.9 |
| Malonic Acid | | | | | 234.7 | 470.9 |
| Maltitol | | | | | | 783.0 |
| Maltose | 1052.6 | 977.1 | | | | 867.8 |
| Maltotriose | 1074.7 | 1276.1 | 54.2 | | | 943.9 |
| Mannan | | | | | | 154.5 |
| Melibionic Acid | | | | | | 270.3 |
| Methyl Pyruvate | | 74.1 | | 870.9 | 818.7 | 611.5 |
| Mono Methyl Succinate | | | | | | 532.1 |
| Mucic Acid | 53.4 | | | | | 162.5 |
| N-Acetyl-D-Galactosamine | | | | | | 59.3 |
| N-Acetyl-D-Glucosamine | | | | 711.9 | | 643.5 |
| N-Acetyl-D-Glucosaminitol | | | | | | 381.9 |
| N-Acetyl-L-Glutamic Acid | | | | | 272.5 | 184.1 |
| N-Acetyl-Neuraminic Acid | | | | | | 647.5 |
| N-Acetyl-β-D-Mannosamine | | | | 375.8 | | 738.3 |
| Oxalic Acid | | | | | | 463.7 |
| Oxalomalic Acid | | 169.4 | 219.2 | | | 691.0 |
| p_Hydroxy Phenyl Acetic Acid | | | | | 987.9 | |
| Palatinose | 129.4 | 197.4 | | | 514.7 | 763.7 |
| Pectin | 199.5 | 683.6 | 141.7 | 68.6 | 334.5 | 996.1 |
| Propionic Acid | | | 58.6 | | 152.1 | 235.6 |
| Putrescine | | | | | 656.8 | 274.2 |
| Pyruvic Acid | 205.5 | 343.3 | 215.7 | 552.9 | 877.9 | 763.0 |
| Quinic Acid | 68.3 | | 92.0 | | 1197.1 | 255.3 |
| Salicin | 74.0 | 222.9 | | | | 146.2 |
| Sebacic Acid | | | | | | 252.2 |
| Sec-Butylamine | 67.6 | | | | | |
| Sedoheptulosan | | | | | | 277.8 |
| Sorbic Acid | | 82.4 | 56.2 | 90.3 | | 495.2 |
| Stachyose | | | | | | 324.4 |
| Succinamic Acid | | | | | 53.6 | 490.3 |
| Succinic Acid | | 241.8 | 125.7 | | 348.4 | 626.1 |
| Sucrose | 1117.8 | 707.3 | | 62.9 | | 940.0 |
| Thymidine | | | | | | 735.9 |
| Tricarballylic Acid | | | | | 510.7 | 177.0 |
| Turanose | 91.9 | 296.1 | | | | 1146.9 |
| Tween 20 | | | | | | 214.4 |
| Tween 40 | | | | 101.2 | | 154.9 |
| Tween 80 | | | | | | 468.6 |
| Tyramine | | | | | 684.5 | |
| Uridine | | | | | | 792.6 |
| Xylitol | | | | | | 280.8 |
| α-D-Glucose | 1068.2 | 1288.0 | 115.5 | 1140.8 | 830.2 | 966.1 |
| α-D-Lactose | | | | | | 764.6 |
| α-Hydroxy-Butyric Acid | | | | | | 567.8 |
| α-Hydroxy-Glutaric Acid γ-Lactone | | | | | | 112.1 |
| α-Keto-Glutaric Acid | | | 160.6 | | 555.1 | 755.7 |
| α-Keto-Valeric Acid | | | | | | 734.6 |
| α-Methyl-D-Galactoside | | | 73.3 | | | 229.9 |
| α-Methyl-D-Glucoside | | | | | | 281.5 |
| α-Methyl-D-Mannoside | | | | | | 264.2 |
| β-D-Allose | | | | | | 122.9 |
| β-Hydroxy Butyric Acid | | | | | | 326.7 |
| β-Methyl-D-Galactoside | | | | | | 358.9 |
| β-Methyl-D-Glucoside | 599.6 | 405.7 | 56.3 | | | 779.9 |
| β-Methyl-D-Glucuronic Acid | | | | | | 400.6 |
| β-Methyl-D-Xyloside | | | | | | 166.1 |
| γ-Amino Butyric Acid | | | | | 1675.2 | |
| γ-Hydroxy Butyric Acid | | | | | | 343.7 |

### Example 3: Rothia Mucilaginosa and Rothia Dentocariosa Inhibit IL-8 Production in HaCat Keratinocytes when Exposed to Lipopolysaccharide or Propionibacterium Acnes

### MATERIALS AND METHODS

### Bacterial Strains, Cell Line and Growth Conditions

Propionibacterium acnes LMG16711 was cultured on reinforced Clostridium agar (RCA; LabM, Heywood, UK) for three days at 37° C. under anaerobic conditions (Anaerogen Compact system [Oxoid, Aalst-Erembodegem, Belgium] or Gaspak EZ system [BD, VWR, Leuven, Belgium]). Liquid cultures of P. acnes were made in Sebomed basal medium and incubated anaerobically for 24 hours. Rothia mucilaginosa DSM20746 and Rothia dentocariosa HVOC18-02 were cultured as described before for the experiments with lung epithelial cells.

HaCaT cells (spontaneously immortalized human keratinocytes) were cultivated in Dulbecco's modified Eagle medium (DMEM; Gibco, Life Technologies Corporation, NY, United States) supplemented with 10% FBS and 1% pen/strep, at 37° C. in a humidified atmosphere containing 5% CO₂.

### Infection

After reaching confluency, HaCaT cells were detached using a 0.02% Trypsine+0.02% EDTA solution and seeded in a 24-well cell culture plate (Greiner Bio-One, Frickenhausen, Germany) at a density of 2.5×10⁴ cells per well and incubated until confluency was reached after 7 days. Fresh medium containing pen/strep was added every two days, and prior to infection medium was changed by antibiotic-free medium. HaCaT cells were exposed to 100 μL LPS (Sigma-Aldrich) or P. acnes at a multiplicity of infection of 10:1, alone or in combination with R. mucilaginosa or R. dentocariosa at an MOI of 10:1. After a 48-hour infection under anaerobic conditions at 37° C., the supernatant was removed and stored at −20° C. for cytokine analysis.

### Cytokine Analysis

IL-8 secretion was measured in the cell culture supernatant by a Human IL-8 ELISA MAX™ Standard assay (Biolegend, San Diego, CA) according to the manufacturer's instructions.

### Statistical Analysis

All experiments were carried out at least in biological triplicate. In vitro infection experiments contained two technical replicates for assessment of the viability and quantification of IL-8 by ELISA. Statistical analysis was performed using SPSS 24.0. The Shapiro-Wilk test was applied to evaluate normality of the data. For normally distributed data, differences between the means were assessed by an independent samples t-test or ANOVA followed by a Dunnett' s post hoc analysis. Not normally distributed data were further analyzed by a Kruskal-Wallis non-parametric test or Mann-Whitney test. Statistical significance is assumed when p-values are <0.05.

### RESULTS

### Exposure of HaCaT Keratinocytes to R. Mucilaginosa or R. Dentocariosa in the Presence of LPS or P. Acnes Does not Induce Cell Death

A HaCaT cell monolayer was exposed to LPS in the presence or absence of R. mucilaginosa or R. dentocariosa at an MOI of 10 for 24 hours. Light microscopic analysis demonstrated that all conditions show a confluent monolayer indicative of a healthy cell population. Bacterial cell clusters can be observed for both bacteria.

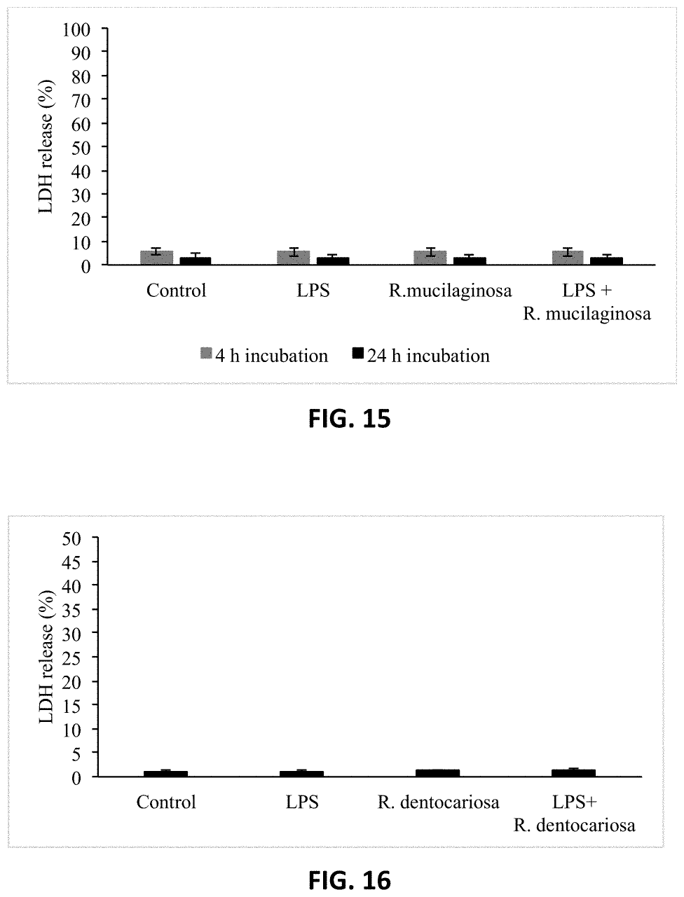

FIGS. 15 and 16 show low LDH release, an indicator for high cell viability, when HaCaT cells were exposed to R. mucilaginosa or R. dentocariosa with or without LPS at different time points, confirming that the tested bacterial strains are not cytotoxic.

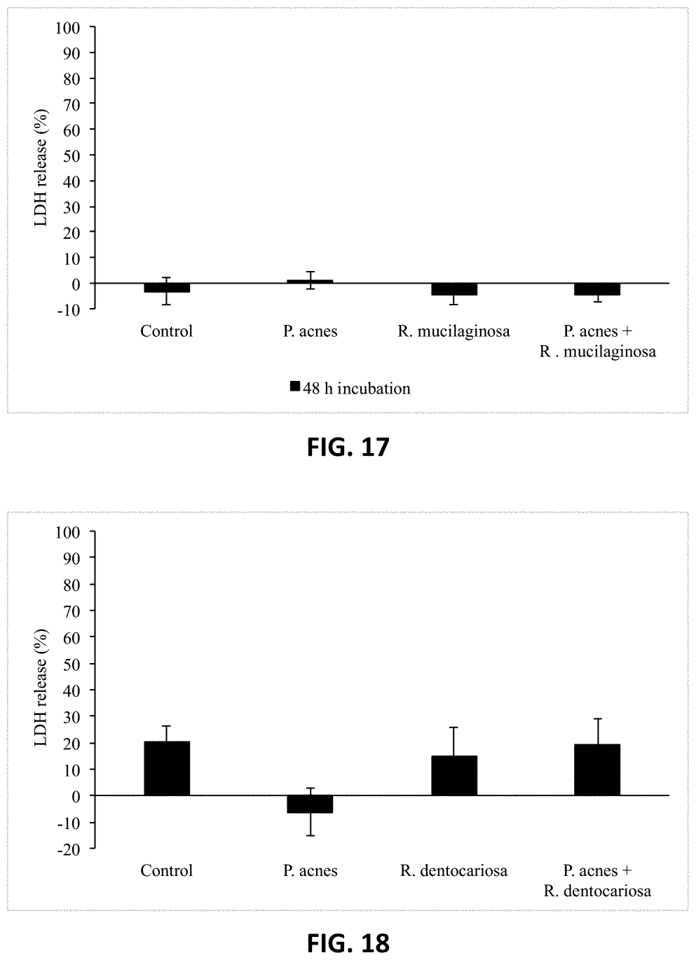

FIGS. 17 and 18 show low LDH release when HaCaT cells are exposed to P. acnes for 48 hours infection in the presence or absence of R. mucilaginosa or R. dentocariosa.

### R. Mucilaginosa and R. Dentocariosa Diminish IL-8 Secretion in HaCaT Keratinocytes when Stimulated by LPS or P. Acnes

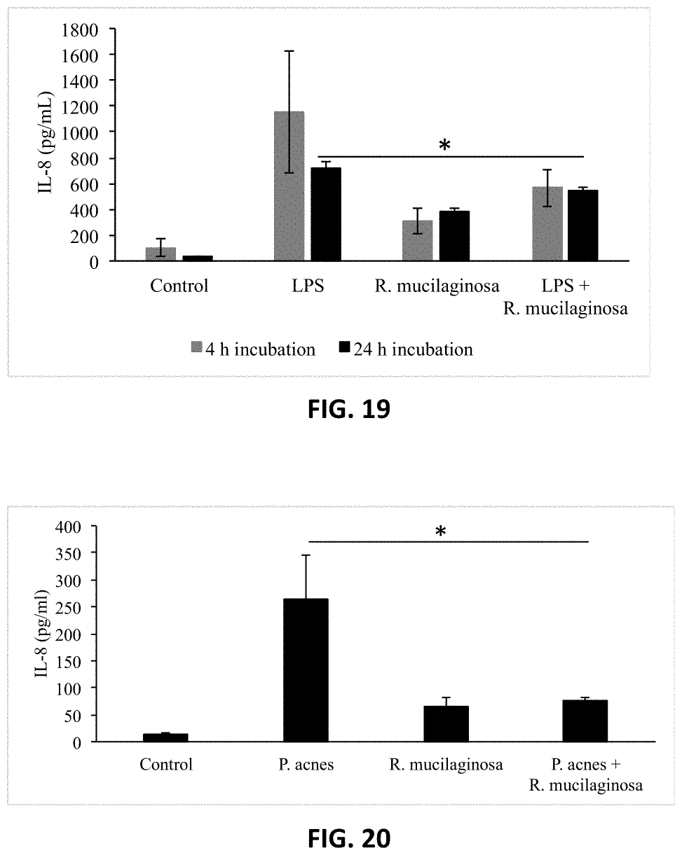

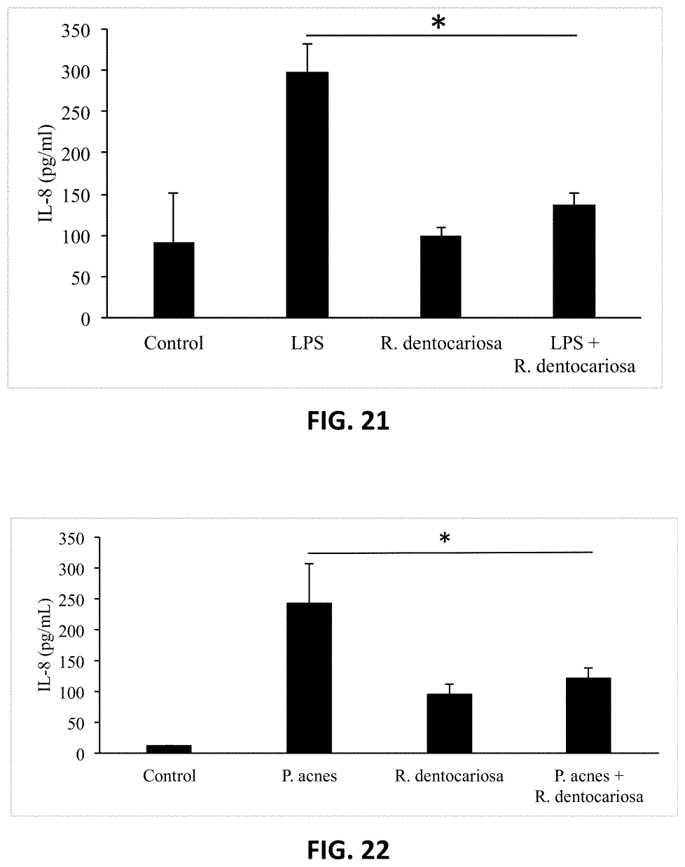

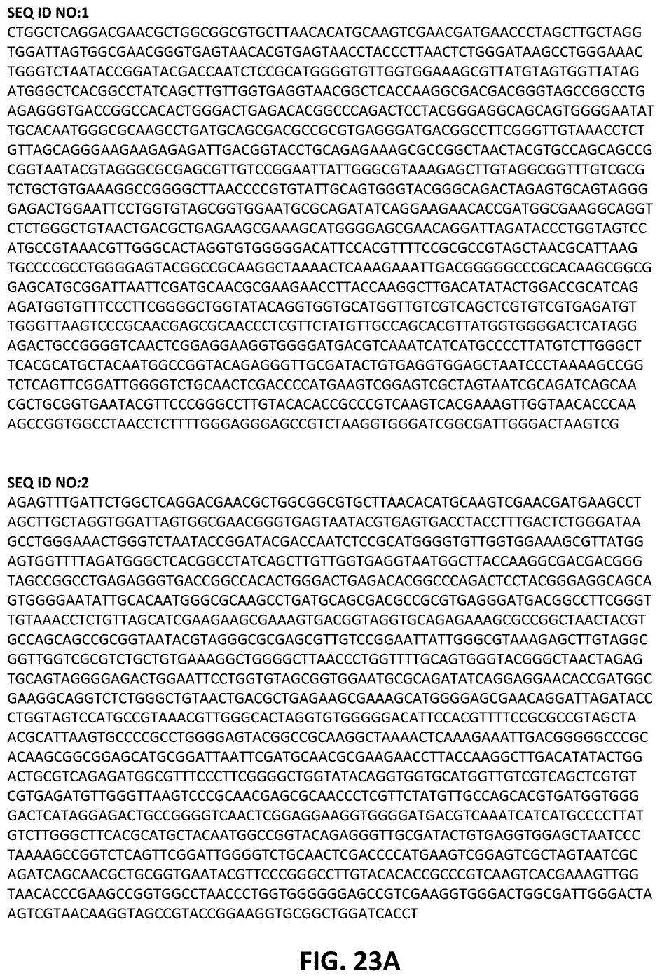

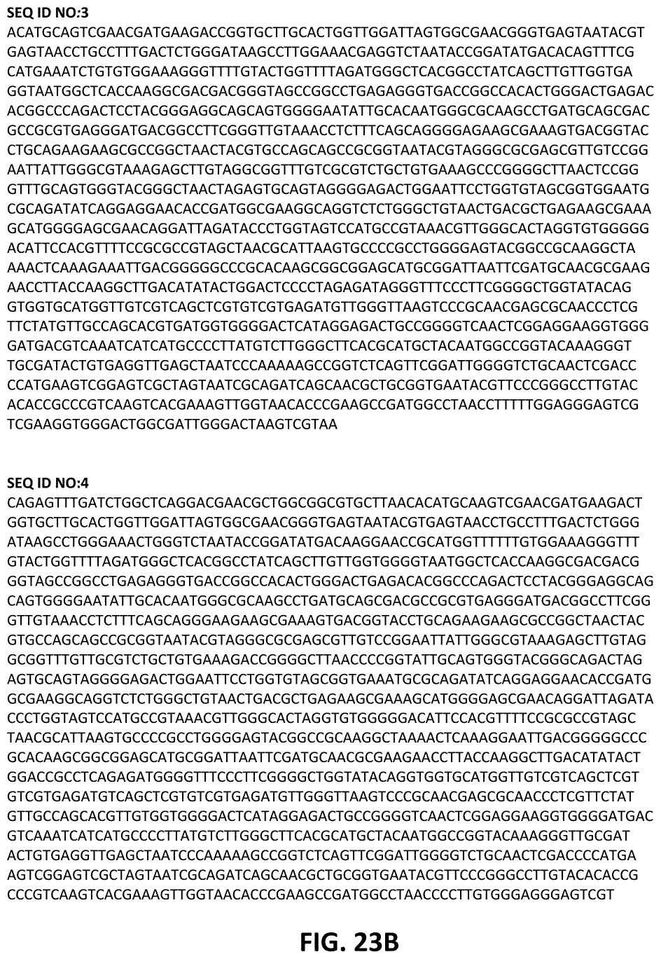

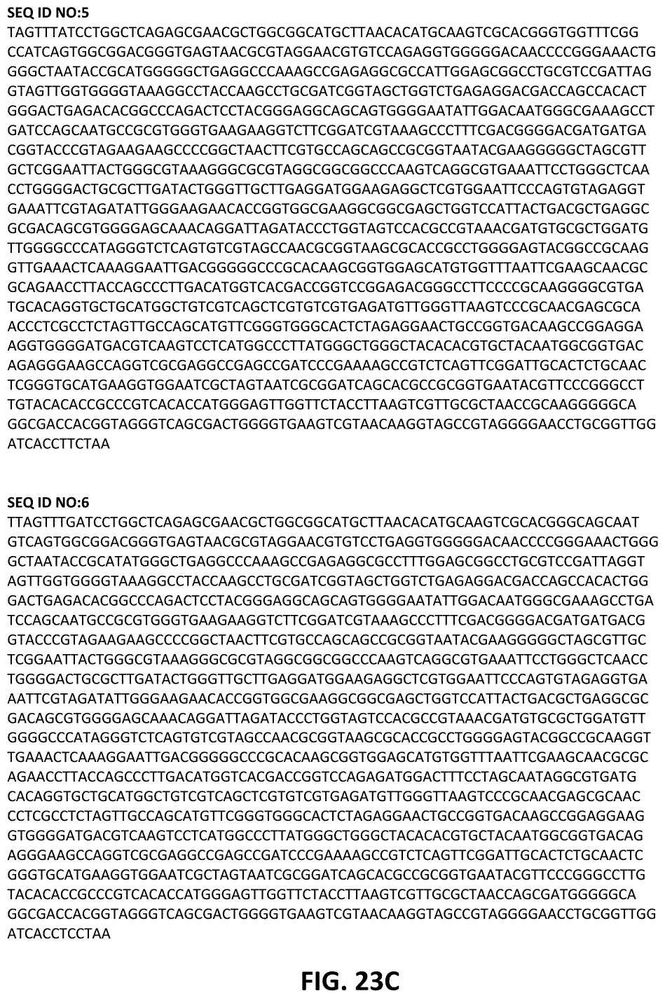

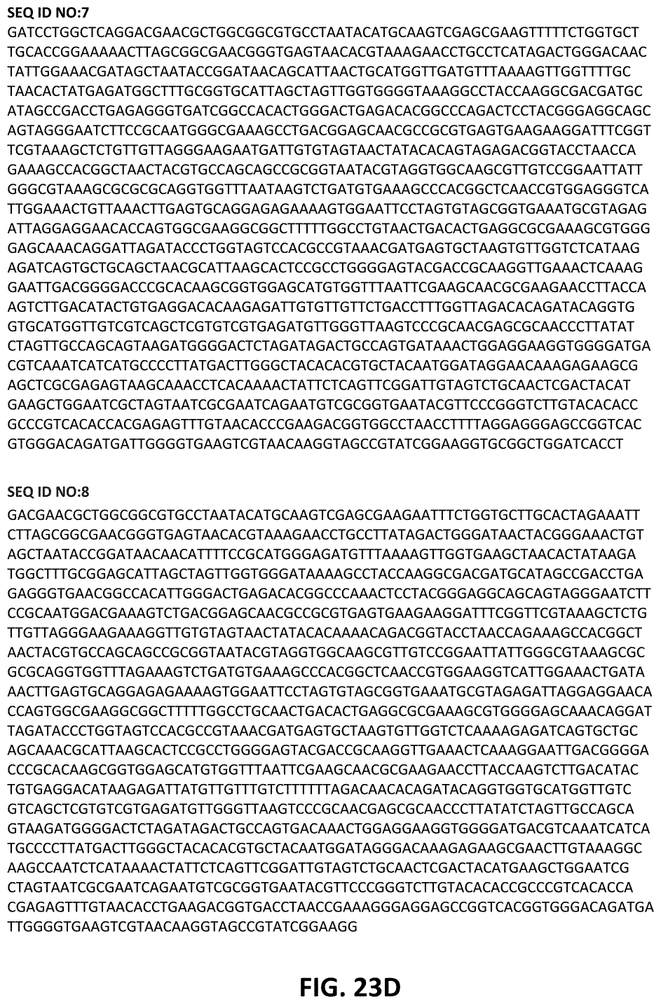

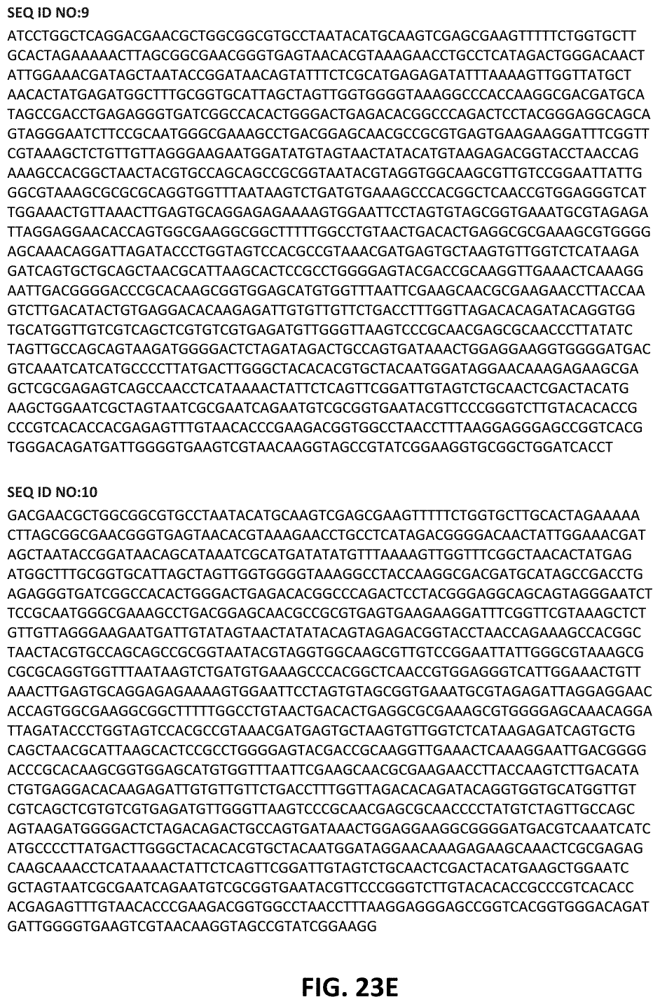

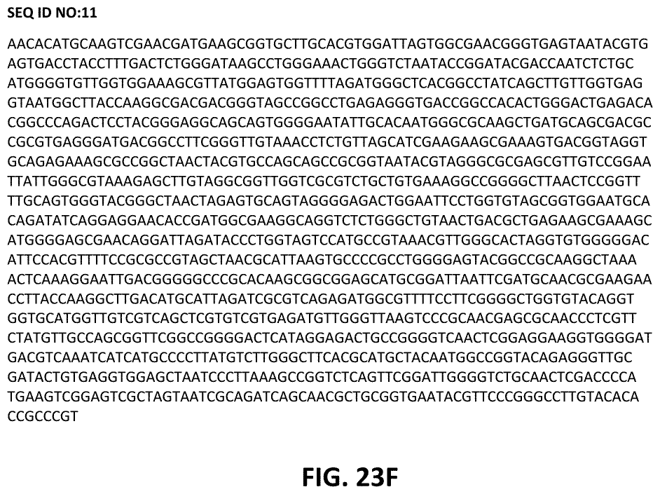

P. acnes induces the production of IL-8 in keratinocytes, leading to the recruitment of neutrophils to the pilosebaceous unit, in turn contributing to inflammation and development of acne. Induction of IL-8 production in HaCaT cells by LPS (FIG. 19) or P. acnes (FIG. 20) is diminished when R. mucilaginosa is added. Similarly, R. dentocariosa also significantly reduced IL-8 production of HaCaT cells when exposed to LPS (FIG. 21) or P. acnes (FIG. 22).

### REFERENCES

Cattoir, L., Coorevits, L., Leroux-Roels, I. et al. Improving timelines in reporting results from positive blood cultures: simulation of impact of rapid identification on therapy on a real-life cohort. Eur. J Clin. Microbiol. Infect. Dis. 37, 2253-2260 (2018).

Cantin, A. M., Hartl, D., Konstan, M. W. & Chmiel, J. F. Inflammation in cystic fibrosis lung disease: Pathogenesis and therapy. J. Cyst. Fibros. 14, 419-430 (2015).

Ind, P. W. COPD disease progression and airway inflammation: uncoupled by smoking cessation. Eur. Respir. J. 26, 764-6 (2005).

Venkataraman, A. et al. Application of a Neutral Community Model To Assess Structuring of the Human Lung Microbiome. (2015) doi:10.1128/mBio.02284-14.

Budden, K. F. et al. Functional effects of the microbiota in chronic respiratory disease. Lancet Respir. Med. (2019) doi:10.1016/S2213-2600(18)30510-1.

Cope, E. K. Host-Microbe Interactions in Airway Disease: toward Disease Mechanisms and Novel Therapeutic Strategies. mSystems. 3(2), (2018).

Yang, X., Li, H., Ma, Q., Zhang, Q. & Wang, C. Neutrophilic Asthma Is Associated with Increased Airway Bacterial Burden and Disordered Community Composition. Biomed Res. Int. 2018: 9230234 (eCollection doi: 10.1155/2018/9230234).

Beasley, V. et al. Lung microbiology and exacerbations in COPD. Int. J. COPD 7, 555-569 (2012).

Vandenbroucke, R. E., Dejonckheere, E. & Libert, C. Series “Matrix metalloproteinases in lung health and disease”: A therapeutic role for matrix metalloproteinase inhibitors in lung diseases? Eur. Respir. J. 38, 1200-1214 (2011).

De Soyza, A. et al. Developing an international Pseudomonas aeruginosa reference panel on behalf of EU FP7 funded COST Action BM1003 &quot; Cell surface virulence determinants of cystic fibrosis pathogens &quot; Microbiologyopen 2, 1010-1023 (2013).

Colvin, K. M. et al. The PeI and PsI polysaccharides provide Pseudomonas aeruginosa structural redundancy within the biofilm matrix. Environ. Microbiol. 14, 1913-1928 (2012).

Vandecandelaere, I. et al. Assessment of Microbial Diversity in Biofilms Recovered from Endotracheal Tubes Using Culture Dependent and Independent Approaches. PLoS One 7, e38401 (2012).

Barrila, J. et al. Organotypic 3D cell culture models: using the rotating wall vessel to study host-pathogen interactions. Nat. Rev. Microbiol. 8, 791-801 (2010).

Crabbe, A., Barrila, J., Mark Ott, C. & Nickerson, C. A. Outpacing Infectious Disease: Mimicking the Host-Pathogen Microenvironment in Three-Dimensions, in Effect of Spaceflight and Spaceflight Analogue Culture on Human and Microbial Cells 93-119 (Springer New York, 2016). doi:10.1007/978-1-4939-3277-1_5.

Carterson, A. J. et al. A549 Lung Epithelial Cells Grown as Three-Dimensional Aggregates : Alternative Tissue Culture Model for Pseudomonas aeruginosa Pathogenesis A549 Lung Epithelial Cells Grown as Three-Dimensional Aggregates : Alternative Tissue Culture Model for Pseudomonas. Infect. Immun. 73, 1129-1140 (2005).

Crabbe, A. et al. Alveolar epithelium protects macrophages from quorum sensing-induced cytotoxicity in a three-dimensional co-culture model. Cell. Microbiol. 13, 469-481 (2011).

Crabbe, A., Barrila, J., Ott, C. M. & Nickerson, C. A. Outpacing Infectious Disease: Mimicking the Host- Pathogen Microenvironment in Three-Dimensions. 93-119 (2008) doi:10.1007/978-1-4939- 3277-1.

Anderson, G. G., Moreau-Marquis, S., Stanton, B. A. & O'Toole, G. A. In vitro analysis of tobramycin- treated Pseudomonas aeruginosa biofilms on cystic fibrosis-derived airway epithelial cells. Infect. Immun. 76, 1423-33 (2008).

Crabbe, A. et al. Antimicrobial efficacy against Pseudomonas aeruginosa biofilm formation in a three-dimensional lung epithelial model and the influence of fetal bovine serum. Sci. Rep. 7, 43321 (2017).

Tran, C. S. et al. The Pseudomonas aeruginosa Type III Translocon Is Required for Biofilm Formation at the Epithelial Barrier. (2014), PLoS Pathog. 10(11): e1004479.

Vandeplassche, E., Coenye, T. & Crabbe, A. Developing selective media for quantification of multispecies biofilms following antibiotic treatment. PLoS ONE 2017; 12(11): e0187540.

Moser, C. et al. Novel experimental Pseudomonas aeruginosa lung infection model mimicking long-term host-pathogen interactions in cystic fibrosis. Apmis 117, 95-107 (2009).

Sonderholm, M. et al. Pseudomonas aeruginosa Aggregate Formation in an Alginate Bead Model System Exhibits In Vivo-Like Characteristics. Appl. Environ. Microbiol. 83, (2017).

Cigana, C. et al. Tracking the immunopathological response to Pseudomonas aeruginosa during respiratory infections OPEN. (2016) doi:10.1038/srep21465.

Serisier, D. J. et al. Effect of Long-term, Low-Dose Erythromycin on Pulmonary Exacerbations Among Patients With Non-Cystic Fibrosis Bronchiectasis. Jama 309, 1260-1267 (2013).

Rogers, G. B. et al. A novel microbiota stratification system predicts future exacerbations in bronchiectasis. Ann. Am. Thorac. Soc. 11, 496-503 (2014).

Taylor, S. L. et al. Long-Term Azithromycin Reduces Haemophilus influenzae and Increases Antibiotic Resistance in Severe Asthma. Am. J. Respir. Crit. Care Med. 200, 309-317 (2019).

Taylor, S. L. et al. Matrix metalloproteinases vary with airway microbiota composition and lung function in non-cystic fibrosis bronchiectasis. Ann. Am. Thorac. Soc. 12, 701-707 (2015).

Cigana, C. et al. Tracking the immunopathological response to Pseudomonas aeruginosa during 542 respiratory infections. Nat. Publ. Gr. (2016) doi:10.1038/srep21465.

Sze, M. A. et al. The lung tissue microbiome in chronic obstructive pulmonary disease. Am. J. Respir. Crit. Care Med. 185, 1073-1080 (2012).

Stein, C. et al. Three dimensional checkerboard synergy analysis of colistin, meropenem, tigecycline against multidrug-resistant clinical Klebsiella pneumonia isolates. PLoS One 10, 1-17 (2015).

Andersen, S. J. et al. Electrolytic membrane extraction enables production of fine chemicals from biorefinery sidestreams. Environ. Sci. Technol. 48, 7135-7142 (2014).

Taylor, S. L. et al. Inflammatory phenotypes in patients with severe asthma are associated with distinct airway microbiology. J. Allergy Clin. Immunol. (2017) doi:10.1016/j.jaci.2017.03.044.

Shah, V. S. et al. Airway acidification initiates host defense abnormalities in cystic fibrosis mice. Science (80-.). 351, 503-507 (2016).

Hunt, J. Airway acidification: Interactions with nitrogen oxides and airway inflammation. Curr. Allergy Asthma Rep. 6, 47-52 (2006).

Odds, F. C. Synergy, antagonism, and what the chequerboard puts between them. J. Antimicrob. Chemother. 52, 1-1 (2003).

Pragman, A. A., Kim, H. B., Reilly, C. S., Wendt, C. & Isaacson, R. E. The Lung Microbiome in Moderate and Severe Chronic Obstructive Pulmonary Disease. PLoS One 7, (2012).

Byun, M. K., Chang, J., Kim, H. J. & Jeong, S. H. Differences of lung microbiome in patients with clinically stable and exacerbated bronchiectasis. PLoS One 12, 1-18 (2017).

Li, M. et al. Pro- and anti-inflammatory effects of short chain fatty acids on immune and endothelial cells. Eur. J. Pharmacol. 831, 52-59 (2018).

Peeters, K. et al. Heterotrophic bacterial diversity in aquatic microbial mat communities from 514 Antarctica. Polar Biol. 35, 543-554 (2012).

Romano-Bertrand, S. et al. Skin microbiota is the main reservoir of Roseomonas mucosa, an emerging opportunistic pathogen so far assumed to be environmental. Clin. Microbiol. Infect. 22, 737.e1-737.e7 (2016).

1-15. (canceled)

16. A method for reducing or treating inflammation in a subject, the method comprising:
administering to the subject a bacterial composition or combination,
wherein the composition or combination comprises at least two bacterial species within the genus Rothia, or
wherein the composition or combination comprises bacteria from the genera
Rothia and Gemella,
Roseomonas and Gemella,
Rothia and Roseomonas, or
Rothia, Roseomonas and Gemella.

17. The method according to claim 16, wherein the subject is suffering from an inflammatory disorder.

18. The method according to claim 16, wherein the subject is suffering from a respiratory disorder or inflammation of the skin.

19. The method according to claim 16, wherein the subject is diagnosed with a disorder selected from the group consisting of: cystic fibrosis (CF), chronic obstructive pulmonary disease (COPD), asthma, non-CF bronchiectasis, bronchitis, sinusitis, sarcoidosis, pneumonia, emphysema, inflammatory bowel disease, mucositis, pulmonary fibrosis, eczema, acne vulgaris, rosacea, seborrheic dermatitis, and psoriasis.

20. The method according to claim 16, wherein the bacteria from the genera are selected from the group consisting of the species: Rothia mucilaginosa, Rothia dentocariosa, Rothia terrae, Rothia amarae, Rothia aeria, Roseomonas gilardii, Roseomonas mucosa, Gemella haemolysans, Gemella asaccharolytic, Gemella bergeri, Gemella cuniculi, Gemella morbillorum, Gemella palaticanis, Gemella parahaemolysans, Gemella sanguinis, and Gemella taiwanensis.

21. The method according to claim 16, wherein the composition or combination is administered to the subject intranasally, by inhalation, by aerosol, topical, dermal, via gavage, rectal, or by oral administration.

22. The method according to claim 16, wherein the bacterial composition or combination is formulated for administration to the subject's respiratory tract or for administration to the skin.

23. The method according to claim 16, wherein the bacterial composition or combination is formulated as a dry powder or a liquid suspension.

24. The method according to claim 16, wherein the bacteria from each genus or species are present the composition or combination in an amount of at least about 10° colony forming units (CFUs) of the bacteria, per unit dosage.

25. The method according to claim 16, wherein the composition or combination comprises live bacteria, fermentation broths, extracts or cell culture supernatants of the bacteria.

26. A pharmaceutical or probiotic composition comprising bacteria of two species from the genus Rothia, or comprising bacterial combinations from the genera Rothia and Gemella, Rothia and Roseomonas, Gemella and Roseomonas or Rothia, Roseomonas and Gemella; and a pharmaceutically acceptable carrier and/or excipient, and wherein
bacteria from the genus Rothia are selected from the group consisting of Rothia mucilaginosa, Rothia dentocariosa, Rothia terrae, Rothia amarae, and Rothia aeria;
bacteria from the genus Gemella are selected from the group consisting of Gemella haemolysans, Gemella asaccharolytic, Gemella bergeri, Gemella cuniculi, Gemella morbillorum, Gemella palaticanis, Gemella parahaemolysans, Gemella sanguinis, and Gemella taiwanensis; and
bacteria from the genus Roseomonas are selected from the group consisting of Roseomonas gilardii and Roseomonas mucosa;
including derivatives or operational taxonomic unit (OTU) encompassing the species.

27. A method for treating or reducing inflammation in a subject, the method comprising:
administering to the subject a composition comprising a compound selected from the group consisting of D,L-glycerol-phosphate, β-methyl-D-glucoside, D-trehalose, glycerol, maltose, maltotriose, N-acetyl-β-D-mannosamine, N-acetyl-D-glucosamine, sucrose, dextrin, oxomalic acid, salicin, turanose, and combinations thereof,
the composition being capable of populating or increasing the microbiota comprising the genera Rothia, Roseomonas and/or Gemella in the subject.

28. The method according to claim 27, wherein the composition further comprises bacteria from the genera Rothia, Roseomonas and/or Gemella.

29. A method for reducing or treating inflammation in a subject, the method comprising administering to the subject a composition comprising bacteria from the genus Gemella.

30. The method according to claim 29, wherein the bacteria are selected from the group consisting of Gemella haemolysans, Gemella asaccharolytic, Gemella bergeri, Gemella cuniculi, Gemella morbillorum, Gemella palaticanis, Gemella parahaemolysans, Gemella sanguinis, and Gemella taiwanensis.

31. The method according to claim 27, wherein the subject is suffering from an inflammatory disorder.

32. The method according to claim 27, wherein the subject is suffering from a respiratory disorder or inflammation of the skin.

33. The method according to claim 29, wherein the subject is suffering from an inflammatory disorder.

34. The method according to claim 29, wherein the subject is suffering from a respiratory disorder or inflammation of the skin.
